(19) United States
(12) Patent Application Publication
(10) Pub. No.: US 2026/0069712 A1
(43) Pub. Date: Mar. 12, 2026

(54) METHODS FOR PURIFICATION OF IONIZABLE LIPIDS

(71) Applicant: MODERNATX, INC., Cambridge, MA (US)

(72) Inventors: Gabor Butora, Cambridge, MA (US); Alison Volkert, Cambridge, MA (US); Dipendra Gyawali, Cambridge, MA (US); Atsushi Endo, Cambridge, MA (US); Zhong Yang, Cambridge, MA (US); Jin Lim, Cambridge, MA (US)

(73) Assignee: MODERNATX, INC., Cambridge, MA (US)

(21) Appl. No.: 19/105,566

(22) PCT Filed: Aug. 22, 2023
(86) PCT No.: PCT/US2023/030761

§ 371 Date: Feb. 21, 2025

Related U.S. Application Data
(60) Provisional application No. 63/400,297, filed on Aug. 23, 2022.

(51) Int. Cl. A61K 48/00; C07C 227/40; C07C 229/06; C07C 229/30; C07D 241/04

(52) U.S. Cl. CPC A61K 48/0033; C07C 227/40; C07C 229/06; C07C 229/30; C07D 241/04

(57) ABSTRACT

Provided herein are methods for purifying an ionizable amino lipid (IAL) composition comprising impurities that may react with polynucleotides, such as mRNA. Methods for purifying IAL compositions comprise one or more scavenging-removal steps to selectively remove reactive impurities.

### CROSS-REFERENCE

This application claims the benefit under 35 U.S.C. § 119(e) of U.S. Provisional Application No. 63/400,297, filed Aug. 23, 2022, the entire contents of which are incorporated herein by reference.

### TECHNICAL FIELD

The present disclosure relates generally to purification of ionizable lipids, such as ionizable lipids that may be used to prepare lipid nanoparticles (LNPs).

### BACKGROUND

The effective delivery of mRNA-based vaccines and therapies is enabled by the use of lipid nanoparticles (LNPs), which protect nucleic acid degradation by exo- and endonucleases and facilitate cellular uptake and expression. Ionizable lipids are an essential component of LNPs. However, due to the labile nature of mRNA, impurities can affect product stability and efficacy. Packer et al., Nature Comm. (2021) 12: 6777, reports the formation of ionizable lipid-mRNA adducts that may be generated by impurities originating in the ionizable lipid component, and reports that the adducts may disrupt mRNA translation and negatively impact the activity of LNP-formulated mRNA products.

Thus, there is a need for methods of purifying ionizable lipids to remove impurities, including impurities that may react with mRNA.

### SUMMARY

The present disclosure relates to the purification of ionizable lipids, such as ionizable amino lipids (IALs), such as may be used in LNPs. Purification may be performed to reduce or eliminate the presence of impurities and other contaminants in a composition comprising ionizable amino lipids, including adduct-forming impurities. The present disclosure further relates to LNPs, mRNA products, and compositions comprising purified ionizable amino lipids.

In at least one aspect, which may be combined with any other aspect or embodiment, the present disclosure relates to a method of purifying an ionizable amino lipid (IAL) present in a first composition comprising the IAL, a first reactive impurity, and a second reactive impurity in a first nonpolar solvent, the method comprising: (a) performing a first scavenging-removal step, comprising: contacting the first composition with a first scavenger, wherein the first scavenger reacts with the first reactive impurity to form a first scavenger-impurity product; and separating the first scavenger-impurity product from the first composition to obtain a second composition comprising the IAL and the second reactive impurity; (b) performing an acid extraction step, comprising contacting the second composition with an acid, wherein the acid reacts with the IAL to form an IAL salt that is soluble in a polar solvent, and transferring the IAL salt and the second reactive impurity into the polar solvent to obtain an IAL salt solution; (c) performing a second scavenging-removal step, comprising: contacting the IAL salt solution with a second scavenger, wherein the second scavenger reacts with the second reactive impurity to form a second scavenger-impurity product; converting the IAL salt into the IAL with a neutralizing agent, wherein the IAL is soluble in a second nonpolar solvent; and separating the IAL from the second scavenger-impurity product, or a derivative thereof formed by the neutralizing agent, by transferring the IAL into the second nonpolar solvent to obtain a purified IAL composition comprising purified IAL.

In some embodiments, the present disclosure relates to a method of purifying an ionizable amino lipid (IAL) present in a first composition comprising the IAL, a reactive halide impurity, and a reactive aldehyde impurity in a first nonpolar solvent, the method comprising: (a) performing a halide scavenging-removal step, comprising: contacting the first composition with a halide scavenger that reacts with the reactive halide impurity to form a scavenger-halide product; and separating the scavenger-halide product from the first composition to obtain a second composition comprising the IAL and reactive aldehyde impurity; (b) performing an acid extraction step, comprising contacting the second composition with an acid that reacts with the IAL to form an IAL salt that is soluble in a polar solvent, and transferring the IAL salt and reactive aldehyde impurity into the polar solvent to obtain an IAL salt solution; (c) performing an aldehyde scavenging-removal step, comprising: contacting the IAL salt solution with an aldehyde scavenger that reacts with the reactive aldehyde impurity to form a scavenger-aldehyde product; converting the IAL salt into the IAL with a neutralizing agent, wherein the IAL is soluble in a second nonpolar solvent; and separating the IAL from the scavenger-aldehyde product, or a derivative thereof formed by the neutralizing agent, by transferring the IAL into the second nonpolar solvent to obtain a purified IAL composition comprising purified IAL.

In some embodiments, the scavenger-aldehyde product or derivative thereof is more soluble in a second polar solvent than in the second nonpolar solvent, and wherein the separating further comprises transferring the scavenger-aldehyde product or derivative thereof into the second polar solvent.

In some embodiments, the neutralizing agent forms a derivative of the scavenger-aldehyde product that is a salt of the scavenger-aldehyde product that is more soluble in the second polar solvent than in the second nonpolar solvent, and wherein the separating comprises transferring the salt of the scavenger-aldehyde product into the second polar solvent. In some embodiments, the salt of the scavenger-aldehyde product is an oxime salt.

In some embodiments, the scavenger-aldehyde product is more soluble in the second polar solvent than in the second nonpolar solvent, and wherein the separating further comprises transferring the second scavenger-impurity product into the second polar solvent.

In some embodiments, the halide scavenger comprises one or more selected from amine compounds, phosphine compounds, thiol compounds, and sulfoxide compounds. In some embodiments, the halide scavenger comprises one or more selected from triphenylphosphine, tributylphosphine, triethylamine, triethylenediamine (DABCO), 4-dimethylaminopyridine (DMAP), dimethylsulfoxide, and dodecanethiol. In some embodiments, the halide scavenger comprises one or more selected from tributylphosphine, triethylamine, triethylenediamine (DABCO), and 4-dimethylaminopyridine (DMAP). In some embodiments, the halide scavenger comprises triethylenediamine (DABCO).

In some embodiments, the scavenger-halide product is a quaternary ammonium compound.

In some embodiments, separating the scavenger-halide product from the first composition comprises performing a liquid-liquid extraction.

In some embodiments, the aldehyde scavenger comprises one or more selected from O-benzylhydroxylamine (O-BHA), N-benzylhydroxylamine, 2-(aminooxy)acetic acid, O-tritylhydroxylamine, 0-(2,3,4,5,6-pentafluorobenzyl)hydroxylamine (PFBHA), p-benzyloxybenzyl alcohol-based hydroxylamines and salts thereof, aminobenzamides, and cysteines and cysteine analogs. In some embodiments, the aldehyde scavenger comprises one or more selected from O-benzylhydroxylamine (O-BHA), N-benzylhydroxylamine, and 2-(aminooxy)acetic acid. In some embodiments, the aldehyde scavenger comprises 2-(aminooxy)acetic acid.

In some embodiments, the scavenger-aldehyde product comprises a compound selected from a Schiff base and an oxime compound.

In some embodiments, the first nonpolar solvent comprises an alkane. In some embodiments, the first nonpolar solvent comprises heptane. In some embodiments, the second nonpolar solvent comprises an alkane. In some embodiments, the second nonpolar solvent comprises heptane. In some embodiments, the first polar solvent comprises acetonitrile, methanol, or a combination thereof. In some embodiments, the second polar solvent comprises acetonitrile, methanol, or a combination thereof.

In some embodiments, the acid comprises one or more selected from carboxylic acids, inorganic acids, and sulfonic acids. In some embodiments, the acid comprises one or more selected from ethylmalonic acid, malonic acid, methanesulfonic acid, methylmalonic acid, and phosphoric acid. In some embodiments, the acid comprises malonic acid.

In some embodiments, the contacting of the halide scavenging-removal step comprises adding the halide scavenger to the first composition and stirring at a temperature of from 60° C. to 90° C. for from 2 hours to 6 hours.

In some embodiments, the halide scavenging-removal step further comprises, after the contacting and before the separating, filtering the composition to remove inorganic salts. In some embodiments, the halide scavenging-removal step further comprises, after the contacting and before the separating, concentrating the first composition.

In some embodiments, the separating of the halide scavenging-removal step comprises washing the first composition with a polar wash solvent to obtain the second composition. In some embodiments, the polar wash solvent comprises acetonitrile, methanol, or a combination thereof.

In some embodiments, the method further comprises removing nonpolar impurities by washing the IAL salt solution with a nonpolar wash solvent before performing the aldehyde scavenging-removal step. In some embodiments, the nonpolar wash solvent for washing the IAL salt solution before performing the aldehyde scavenging-removal step comprises heptane.

In some embodiments, the contacting of the aldehyde scavenging-removal step comprises adding the aldehyde scavenger to the IAL salt solution and stirring at a temperature of 20° C. to 50° C. for from 15 minutes to 4 hours.

In some embodiments, the aldehyde scavenging-removal step further comprises, after the contacting and before the converting, washing the IAL salt solution comprising the aldehyde-scavenger product with a nonpolar wash solvent. In some embodiments, the nonpolar wash solvent for washing the IAL salt solution, after the contacting and before the converting, comprises heptane.

In some embodiments, the separating of the aldehyde scavenging-removal step comprises washing the second nonpolar solvent containing the IAL with a polar wash solvent to obtain the purified IAL composition comprising purified IAL. In some embodiments, the polar wash solvent for washing the second nonpolar solvent containing the IAL comprises one or more of acetonitrile and methanol.

In some embodiments, the method further comprises filtering the purified IAL composition to remove residual impurities. In some embodiments, the filtering comprises passing the purified IAL composition through a silica gel plug.

In some embodiments, the method further comprises concentrating the purified IAL composition.

In at least one aspect, which may be combined with any other aspect or embodiment, the present disclosure relates to a method of purifying an ionizable amino lipid (IAL) present in a first composition comprising the IAL and a reactive halide impurity in a nonpolar solvent, the method comprising performing a halide scavenging-removal step, comprising: contacting the first composition with a scavenger that reacts with the reactive halide impurity to form a scavenger-halide product; and separating the scavenger-halide product from the first composition to obtain a purified IAL composition comprising purified IAL.

In at least one aspect, which may be combined with any other aspect or embodiment, the present disclosure relates to a method of purifying an ionizable amino lipid (IAL) present in a composition comprising the IAL and a reactive aldehyde impurity in a first nonpolar solvent, the method comprising: performing an acid extraction step, comprising contacting the composition with an acid that reacts with the IAL to form an IAL salt that is soluble in a polar solvent, and transferring the IAL salt and reactive aldehyde impurity into the polar solvent to obtain an IAL salt solution; and performing an aldehyde scavenging-removal step, comprising: contacting the IAL salt solution with an aldehyde scavenger that reacts with the reactive aldehyde impurity to form a scavenger-aldehyde product; converting the IAL salt into the IAL with a neutralizing agent, wherein the IAL is soluble in a second nonpolar solvent; and separating the IAL from the scavenger-aldehyde product, or a derivative thereof formed by the neutralizing agent, by transferring the IAL into the second nonpolar solvent to obtain a purified IAL composition comprising purified IAL. In some embodiments, the derivative of the second scavenger-impurity product is a salt of the scavenger-impurity product.

In some embodiments, the IAL comprises a compound according to Formula (I):

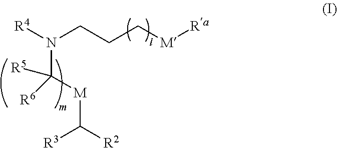

or an isomer thereof, wherein
R′^{a} is R′^{branched}; wherein R′^{branched} is:

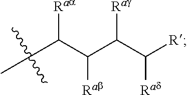

wherein [symbol] denotes a point of attachment; wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

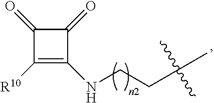

wherein [symbol] denotes a point of attachment;
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10; each R⁵ is independently selected from the group consisting of C₁₋₃ alkyl, C₂₋₃ alkenyl, and H; each R⁶ is independently selected from the group consisting of C₁₋₃ alkyl, C₂₋₃ alkenyl, and H; M and M′ are each independently selected from the group consisting of —C(O)O— and —OC(O)—; R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the IAL is selected from:

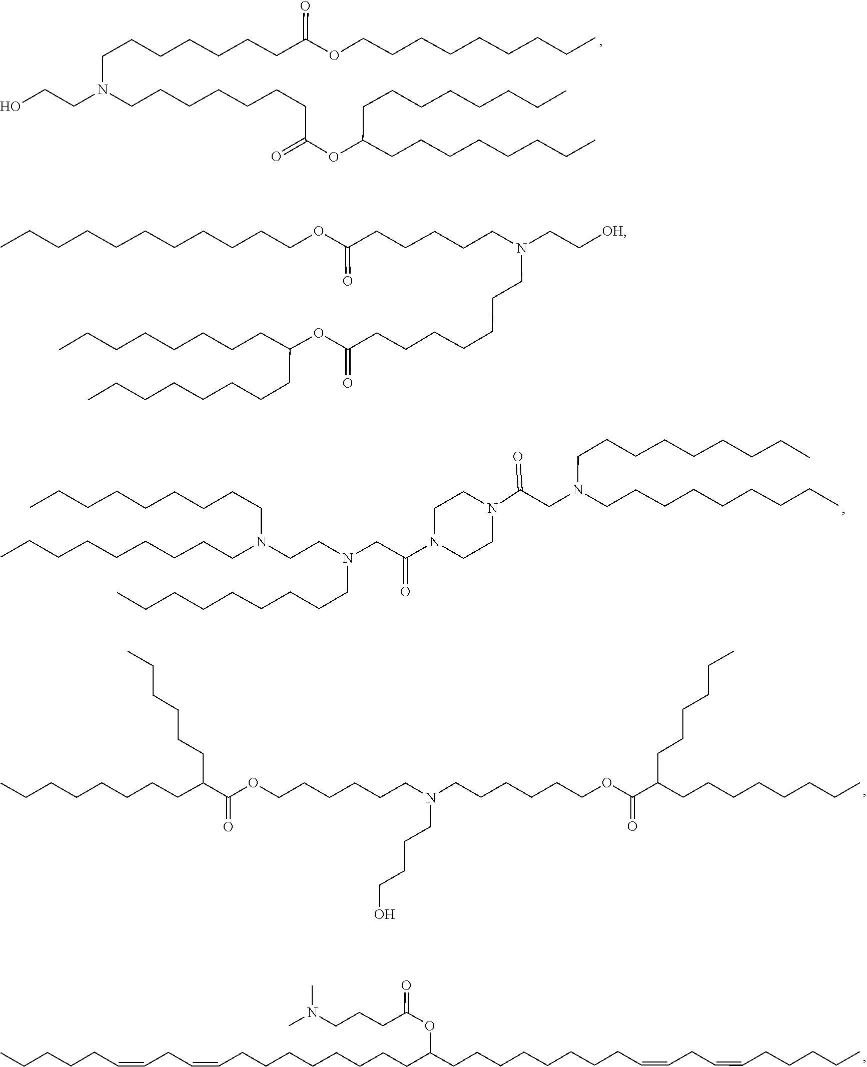

In some embodiments, the method does not comprise performing column chromatography.

In at least one aspect, which may be combined with any other aspect or embodiment, the present disclosure relates to a purified ionizable amino lipid (IAL) obtained by any of the methods disclosed herein.

In at least one aspect, which may be combined with any other aspect or embodiment, the present disclosure relates to a lipid nanoparticle (LNP) comprising a purified ionizable amino lipid obtained by any of the methods disclosed herein. In some embodiments, the LNP further comprises a polynucleotide. In some embodiments, the polynucleotide is mRNA. In some embodiments, the LNP further comprises a phospholipid, cholesterol, and a PEG-lipid. In some embodiments, the LNP comprises a molar ratio of 20-60% IAL, 5-25% phospholipid, 25-55% cholesterol, and 0.5-15% PEG-lipid, based on lipid components.

In at least one aspect, which may be combined with any other aspect or embodiment, the present disclosure relates to a pharmaceutical composition comprising a lipid nanoparticle (LNP), wherein the LNP comprises a purified ionizable amino lipid obtained by any of the methods disclosed herein. In some embodiments, the LNP further comprises a polynucleotide. In some embodiments, the polynucleotide is mRNA. In some embodiments, the LNP further comprises a phospholipid, cholesterol, and a PEG-lipid. In some embodiments, the LNP comprises a molar ratio of 20-60% IAL, 5-25% phospholipid, 25-55% cholesterol, and 0.5-15% PEG-lipid, based on lipid components.

In at least one aspect, which may be combined with any other aspect or embodiment, the present disclosure relates to a pharmaceutical composition, comprising: a lipid nanoparticle comprising a mRNA, a phospholipid, a cholesterol, and a PEG-lipid, and a purified ionizable amino lipid (IAL) obtained by any of the methods disclosed herein. In some embodiments, less than about 10% of the mRNA is in the form of an ionizable lipid-polynucleotide adduct impurity, optionally wherein less than about 5% of the mRNA is in the form of an ionizable lipid-polynucleotide adduct impurity, optionally wherein less than about 1% of the mRNA is in the form of an ionizable lipid-polynucleotide adduct impurity, optionally wherein the pharmaceutical composition is substantially free of ionizable lipid-polynucleotide adduct impurity, as measured by reverse phase ion pair high performance liquid chromatography (RP-IP HPLC).

In some embodiments, the IAL is selected from:

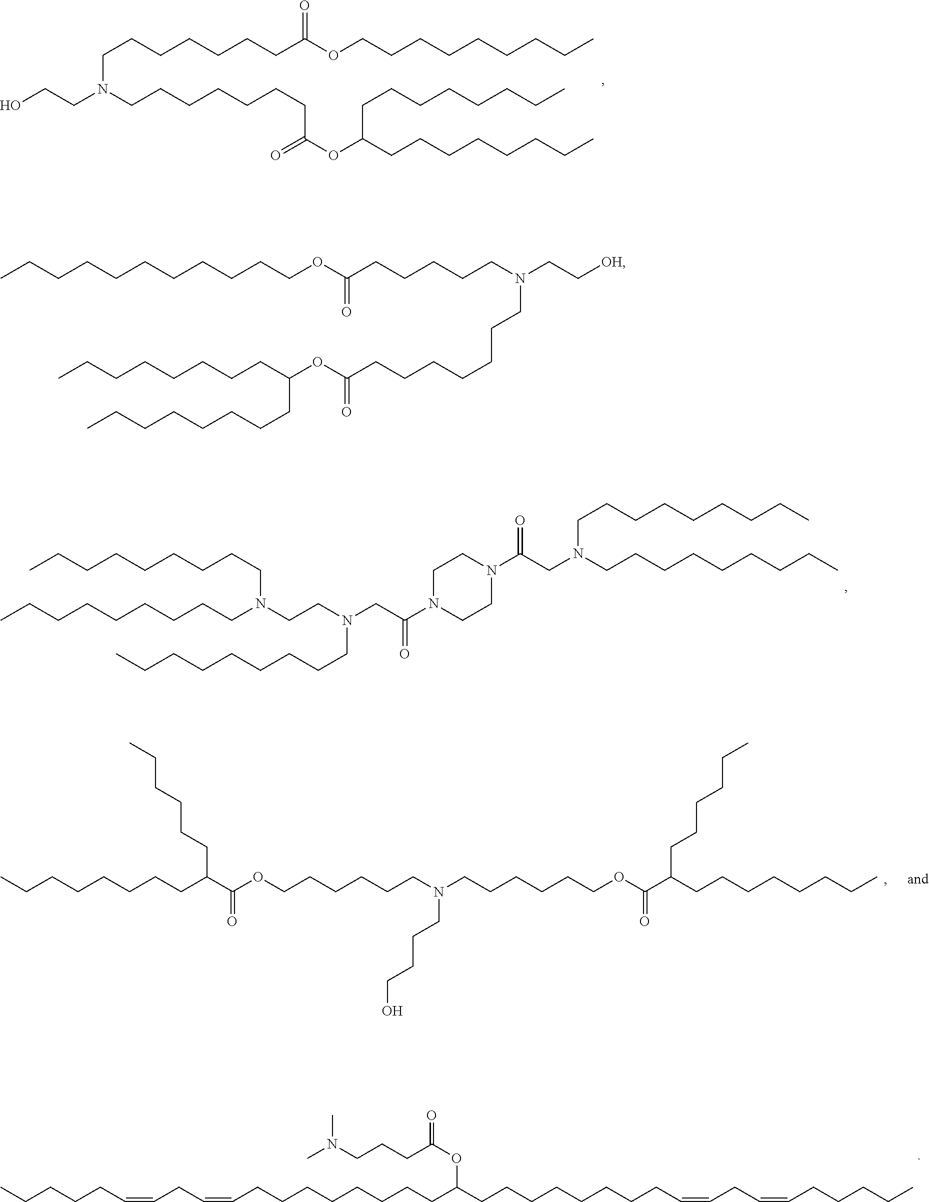

In some embodiments, the composition comprises a buffer selected from the group consisting of sodium phosphate, sodium citrate, sodium succinate, histidine, histidine-HCl, sodium malate, sodium carbonate, and Tris (tris(hydroxymethyl)aminomethane). In some embodiments, the composition comprises a cryoprotectant. In some embodiments, the cryoprotectant is selected from the group consisting of: mannitol; sucrose; trehalose; lactose; glycerol; dextrose; and combinations thereof. In some embodiments, the ionizable lipid-polynucleotide adduct impurity comprises an aldehyde-mRNA adduct impurity. In some embodiments, an amount of lipid aldehydes in the composition is less than about 50 ppm. In some embodiments, the composition comprises Tris buffer and sucrose. In some embodiments, the LNP in the composition comprises a molar ratio of 20-60% IAL, 5-25% phospholipid, 25-55% cholesterol, and 0.5-15% PEG-lipid, based on lipid components.

In at least one aspect, which may be combined with any other aspect or embodiment, the present disclosure relates to a composition comprising a lipid nanoparticle comprising a mRNA, a phospholipid, a cholesterol, a PEG-lipid, and an IAL comprising a tertiary amine group obtained by the method according to any of the methods disclosed herein, wherein less than about 10% of the mRNA is in the form of an ionizable lipid-polynucleotide adduct impurity, optionally wherein less than about 5% of the mRNA is in the form of an ionizable lipid-polynucleotide adduct impurity, optionally wherein less than about 1% of the mRNA is in the form of am ionizable lipid-polynucleotide adduct impurity, optionally wherein the pharmaceutical composition is substantially free of ionizable lipid-polynucleotide adduct impurity, as measured by reverse phase ion pair high performance liquid chromatography (RP-IP HPLC).

In some embodiments, the IAL is selected from:

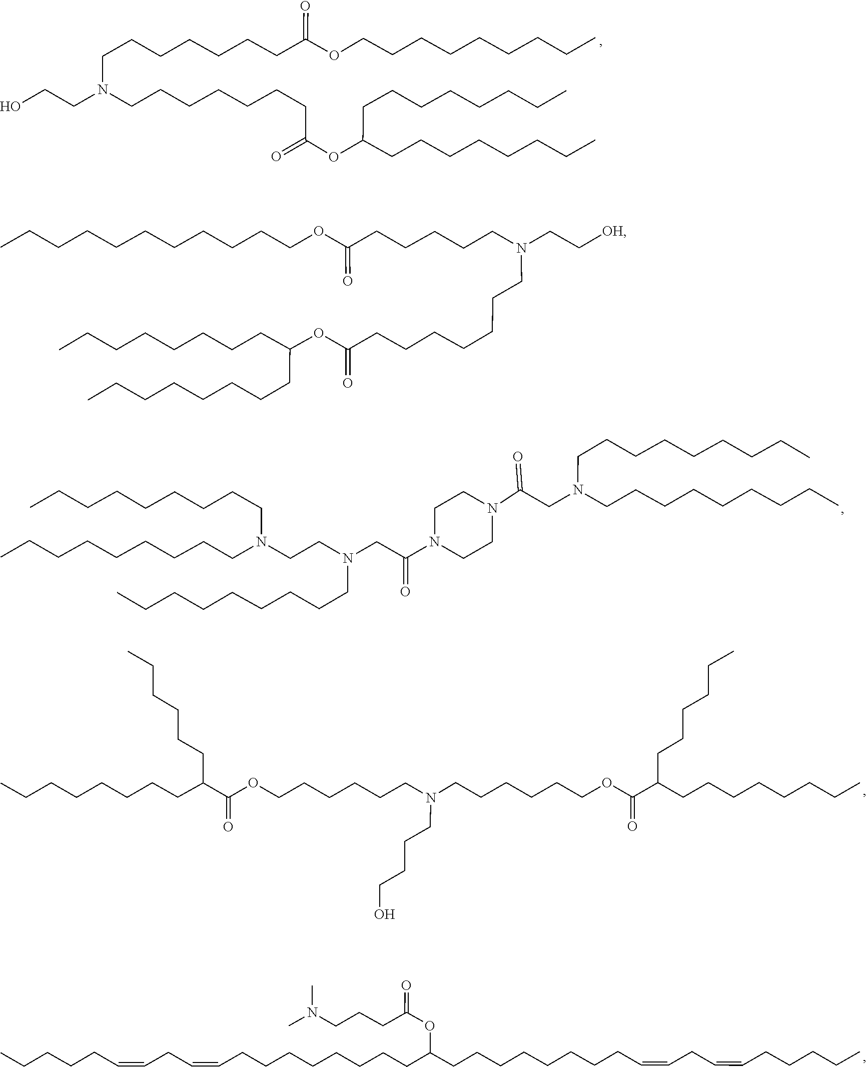

In some embodiments, the composition comprises a buffer selected from the group consisting of sodium phosphate, sodium citrate, sodium succinate, histidine, histidine-HCl, sodium malate, sodium carbonate, and Tris (tris(hydroxymethyl)aminomethane). In some embodiments, the composition comprises a cryoprotectant. In some embodiments, the cryoprotectant is selected from the group consisting of: mannitol; sucrose; trehalose; lactose; glycerol; dextrose; and combinations thereof. In some embodiments, the ionizable lipid-polynucleotide adduct impurity comprises an aldehyde-mRNA adduct impurity. In some embodiments, an amount of lipid aldehydes in the composition is less than about 50 ppm. In some embodiments, the composition comprises Tris buffer and sucrose. In some embodiments, the LNP in the composition comprises a molar ratio of 20-60% IAL, 5-25% phospholipid, 25-55% cholesterol, and 0.5-15% PEG-lipid, based on lipid components.

In at least one aspect, which may be combined with any other aspect or embodiment, the present disclosure relates to a composition comprising a lipid nanoparticle comprising a mRNA, a phospholipid, a cholesterol, a PEG-lipid, and an IAL obtained by the method according to any one of claims 1-49, wherein the ionizable lipid is selected from:

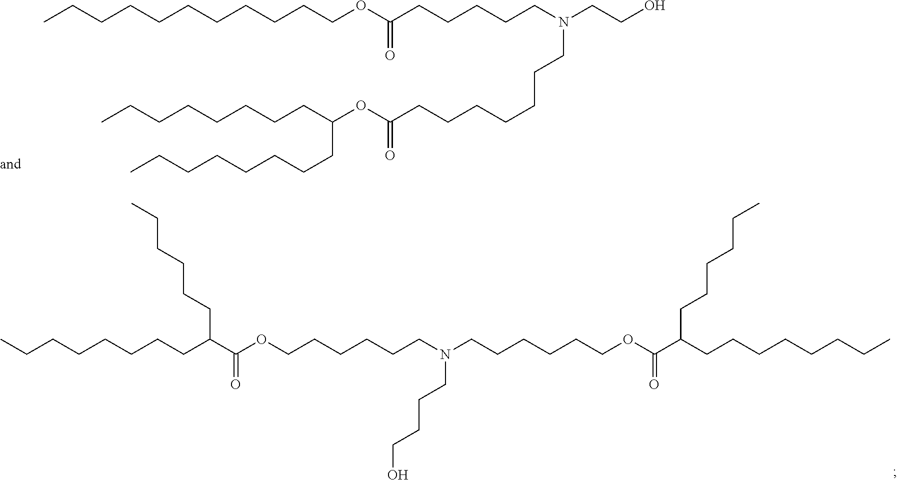

wherein the composition comprises a Tris buffer and sucrose; and wherein less than about 10% of the mRNA is in the form of an ionizable lipid-polynucleotide adduct impurity, optionally wherein less than about 5% of the mRNA is in the form of the ionizable lipid-polynucleotide adduct impurity, optionally wherein less than about 1% of the mRNA is in the form of the ionizable lipid-polynucleotide adduct impurity, optionally wherein the pharmaceutical composition is substantially free of ionizable lipid-polynucleotide adduct impurity, as measured by reverse phase ion pair high performance liquid chromatography (RP-IP HPLC).

It should be appreciated that all combinations of the foregoing aspects and additional aspects discussed in greater detail below are contemplated as being part of the subject matter disclosed herein (provided such concepts are not mutually exclusive or inconsistent).

### BRIEF DESCRIPTION OF THE DRAWINGS

FIG. 1 is schematic illustration of a method for purifying ionizable amino lipids as disclosed herein.

FIG. 2 is a schematic illustration of a halide scavenging-removal process as disclosed herein.

FIG. 3 is a schematic illustration of an acid extraction as disclosed herein.

FIG. 4 is a schematic illustration of an aldehyde scavenging-removal process as disclosed herein.

FIG. 5 is a plot of lipid-mRNA adduct (“binary”) formation for IAL recovered after various steps in an embodiment of the IAL purification process disclosed herein.

FIG. 6 is a plot of lipid-mRNA adduct (“binary”) formation for purified IAL obtained by embodiments of an IAL purification process as described herein, including halide and aldehyde scavenging steps (0.4%), no scavenging steps (5.8%), only an aldehyde scavenging step (no halide scavenging) (144%), or only a halide scavenging step (no aldehyde scavenging) (3.6%).

Reference is made to the accompanying drawings throughout the following detailed description, however the illustrative implementations described in the detailed description and illustrated in the drawings are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a variety of different configurations, all of which are explicitly contemplated and made part of this disclosure.

### DETAILED DESCRIPTION

After synthesis of ionizable lipids (e.g., ionizable amino lipids, referred to herein as IALs), reactive impurities (e.g., alkyl halides and carbonyl-containing compounds such as aldehydes and ketones) may be present as side-products or remaining reagents, and may be carried forward when the ionizable lipids are used to prepare lipid nanoparticles (LNPs). For example, ionizable lipids (e.g., IALs) may be prepared by methods as disclosed in WO 2017/049245, which may result in a composition that comprises the ionizable lipid along with reactive impurities (e.g., alkyl halides and carbonyl-containing compounds such as aldehydes and ketones). Even if these impurities are present at low levels or below the limit of detection by conventional techniques (e.g., UPLC-CAD), they can react with nucleophilic moieties present on polynucleotides such as mRNA, and form adducts which may reduce the activity of, e.g., mRNA LNP products.

Current methods for removing reactive impurities and achieving high UPLC-CAD purity for ionizable lipids may include reductive treatment as a separate batch reaction to deactivate carbonyl species, followed by a series of washes. Column chromatography is implemented to remove halides and other process impurities. Although this methodology is robust on a manufacturing scale, commercial-scale normal phase chromatography can limit process throughput, increase organic solvent usage and waste generation, and lead to increased production time and costs. Thus, eliminating column chromatography from the process stream could afford significant cost savings. The methods disclosed herein can remove reactive impurities and achieve high ionizable lipid (e.g., IAL) purity (as may be assessed by UPLC-CAD or more sensitive techniques such as RP-IP-HPLC) without a column chromatography step. Thus, in some embodiments, a method of purifying ionizable lipids (e.g., IALs) as disclosed herein (in accordance with any of the various disclosed embodiments) does not comprise performing column chromatography.

The present disclosure provides methods of purifying ionizable lipids from reactive impurities such as reactive halides (e.g., alkyl halides) and carbonyl-containing compounds such as aldehydes and ketones. The ionizable lipids typically have a tertiary amine group; such an ionizable lipid is referred to herein as an ionizable amino lipid (IAL).

In at least one aspect, methods of purifying IALs as described herein comprise one or more scavenging-removal processes to eliminate the presence of or reduce the concentration of one or more reactive impurities (e.g., aldehydes and halides) present in a composition comprising an IAL or IAL salt. The scavenging may comprise contacting a composition comprising an IAL and one or more reactive impurities with a scavenger (e.g., an aldehyde scavenger or a halide scavenger) which reacts with the reactive impurity and forms a scavenger-impurity product. The scavenging leverages selective reactivity of the scavenger with reactive impurities (and not with the IAL). The resulting scavenger-impurity product may have different physical and/or chemical properties (e.g., solubility) that facilitate separation from the IAL (e.g., by liquid-liquid extraction).

In some embodiments, separation is performed by liquid-liquid extraction and/or a series of washes to separate scavenger-impurity products from the IAL. In some embodiments, the removal comprises a liquid-liquid extraction that retains the IAL or salt thereof in a first solvent and retains one or more reactive impurities or scavenger-impurity products in a second solvent that may be separated from the first solvent, e.g., because the first solvent and second solvent are immiscible. Thus, the removal may rely on differences in physical and/or chemical properties of the reactive impurities or the scavenger-impurity products versus those of the IAL or salts thereof, such as differences in solubility.

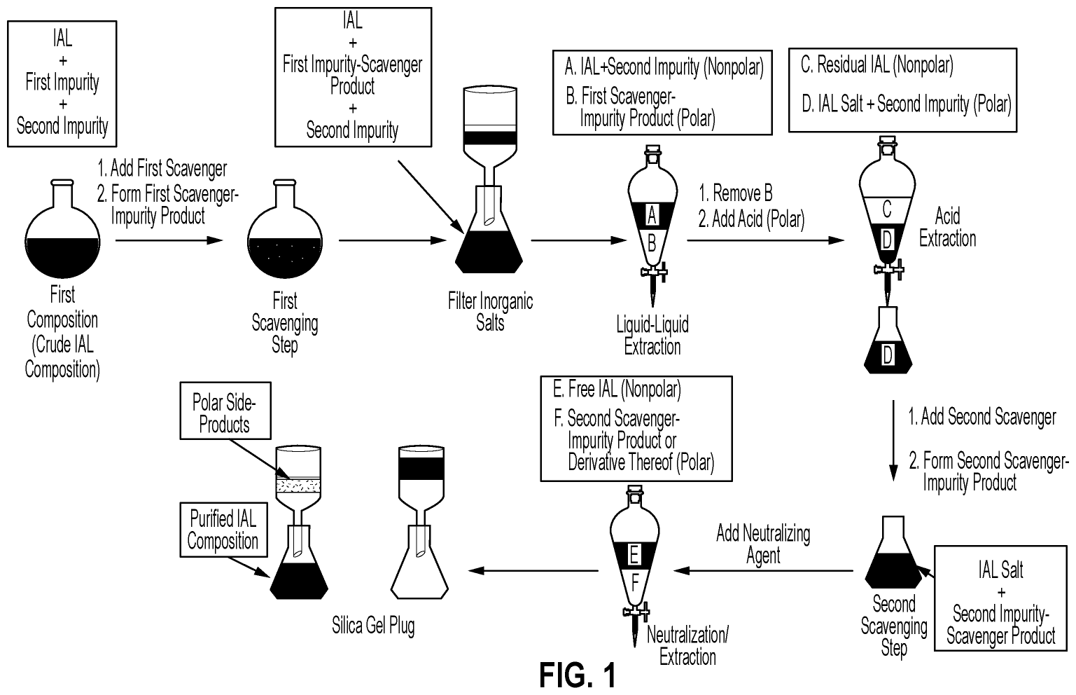

FIG. 1 is a schematic illustration of an exemplary method of purifying an IAL composition comprising an IAL. An IAL composition to be purified may be obtained directly after synthesis of one or more IALs, or may be subject to one or more purification steps prior to purification by the methods disclosed herein. Typically, the IAL composition to be purified comprises the IAL and one or more reactive impurities (e.g., a first reactive impurity, a second reactive impurity, etc.) in a first solvent. In some embodiments, the IAL is soluble in nonpolar solvents (e.g., heptane), so the first solvent may be a nonpolar solvent.

To eliminate the presence of or reduce the concentration of a first impurity (e.g., an alkyl halide), a first scavenger is added to the composition comprising the IAL (the “first” composition”). The first scavenger selectively reacts with the first impurity (and not the IAL) to form a first scavenger-impurity product, which has a different solubility from that of the IAL. That is, the IAL may be soluble in nonpolar solvents but less soluble in polar solvents, whereas the first scavenger-impurity product may be soluble in polar solvents but less soluble in nonpolar solvents. Thus, the first scavenger-impurity product may be separated from the first composition by liquid-liquid extraction (e.g., in a separatory funnel) by washing the composition with a polar solvent. This extraction removes the first scavenger-impurity product in the polar solvent, while the IAL (and other components that may be present, including additional reactive impurities) remain in the first solvent (e.g., a nonpolar solvent such as heptane). Thus, the first scavenging-removal step produces a second composition comprising the IAL and other components that may be present, such as a second reactive impurity, in a solvent (e.g., a nonpolar solvent such as heptane). Referring still to FIG. 1, in some embodiments, the second composition comprises the IAL and a second reactive impurity, while the first reactive impurity is absent or is present at a lower concentration than in the first composition.

In some embodiments, a first composition comprising the IAL contains inorganic salts, such as inorganic salts which may be residual from the synthesis of the IAL (e.g., from an alkylation reaction conducted to prepare the IAL), such as carbonate salts such as potassium carbonate or halide salts such as potassium iodide. Inorganic salts may be removed from the composition by filtration. If conducted, this optional filtration step may be performed before or after removing the first scavenger-impurity product, for example, before or after a liquid-liquid extraction that separates and removes the first scavenger-impurity product from the first composition.

Referring still to FIG. 1, removing the second reactive impurity may be effected by a process that comprises altering the solubility of the IAL to facilitate a second scavenging-removal process. In some embodiments, altering the solubility of the IAL comprises converting the IAL (a tertiary amine soluble in nonpolar solvents) into an IAL salt soluble in polar solvents (e.g., an ammonium salt soluble in polar solvents), such as by performing an acid extraction step. In some embodiments, performing an acid extraction comprises contacting the second composition comprising the IAL with an acid. By contacting the second composition with an acid (e.g., a dicarboxylic acid), the IAL forms an IAL salt that has a different solubility from the IAL. In some embodiments, the IAL is more soluble in nonpolar solvents (e.g., heptane) and less soluble in polar solvents as compared to the IAL salt, while the IAL salt is more soluble in polar solvents (e.g., acetonitrile, methanol, or combinations thereof) and less soluble in nonpolar solvents as compared to the IAL. In some embodiments, the acid extraction step comprises contacting the second composition comprising the IAL and the second reactive impurity with an acid, thereby converting the IAL into an IAL salt soluble in a polar solvent, and transferring the IAL salt and the second reactive impurity into the polar solvent, such as by washing the composition comprising the IAL salt with a polar solvent (e.g., acetonitrile, methanol, or combinations thereof), to produce an IAL salt composition comprising the IAL salt and the second reactive impurity in the polar solvent.

In some embodiments, the IAL salt composition is washed with a nonpolar solvent (e.g., heptane) to remove nonpolar impurities, such as non-ionizable impurities and impurities that do not contain an amine (e.g., residual alkyl halides, aldehydes, etc.).

To reduce the concentration of the second reactive impurity (e.g., an aldehyde), a second scavenger can be added to the IAL salt composition. The second scavenger selectively reacts with the second reactive impurity (and not the IAL salt) to form a second scavenger-impurity product. Subsequently, a neutralizing agent can be introduced to convert the IAL salt into the IAL, which is soluble in nonpolar solvents. In some embodiments, the IAL is more soluble in nonpolar solvents (e.g., heptane) than in polar solvents (e.g., water, acetonitrile, methanol, or combinations thereof), while the second scavenger-impurity product is more soluble in polar solvents (e.g., water, acetonitrile, methanol, or combinations thereof) than in nonpolar solvents (e.g., heptane). In some embodiments, the neutralizing agent may also convert the second scavenger-impurity product into a derivative of the second scavenger-impurity product which is more soluble in polar solvents than in nonpolar solvents, such as, but not limited to, a salt of the second scavenger-impurity product. In some embodiments, the IAL is more soluble in nonpolar solvents (e.g., heptane) and less soluble in polar solvents as compared to the derivative (e.g., salt) of the second scavenger-impurity product, while the derivative of the second scavenger-impurity product is more soluble in polar solvents (e.g., water, acetonitrile, methanol, or combinations thereof) and less soluble in nonpolar solvents as compared to the IAL. Thus, the IAL may be separated from the second scavenger-impurity product by a process comprising a neutralization reaction followed by transferring the IAL into a nonpolar solvent (e.g., by liquid-liquid extraction) to produce an IAL solution comprising the IAL in a nonpolar solvent (e.g., heptane), wherein the second reactive impurity is absent from the IAL solution or is present at a lower concentration than in the first composition. The second scavenger-impurity product or derivative thereof may be further separated from the IAL solution, such as by washing the IAL solution with a polar solvent (e.g., acetonitrile, methanol, and combinations thereof), to obtain a purified IAL composition comprising purified IAL.

The purified IAL composition optionally may be filtered over a porous medium (e.g., a silica plug) to remove residual impurities. In some embodiments, the impurities removed by filtration over a porous medium are polar impurities (e.g., secondary amines, carboxylic acids, etc.). Additionally or alternatively, the purified IAL composition optionally may be concentrated by removing the nonpolar solvent (e.g., by rotary evaporation).

Thus, in one aspect, the present disclosure relates to methods of purifying an ionizable amino lipid (IAL) present in a first composition comprising the IAL, a first reactive impurity, and a second reactive impurity in a first nonpolar solvent, comprising: (a) performing a first scavenging-removal step, comprising: contacting the first composition with a first scavenger, wherein the first scavenger reacts with the first reactive impurity to form a first scavenger-impurity product; and separating the first scavenger-impurity product from the first composition to obtain a second composition comprising the IAL and the second reactive impurity; (b) performing an acid extraction step, comprising contacting the second composition with an acid, wherein the acid reacts with the IAL to form an IAL salt that is soluble in a polar solvent, and transferring the IAL salt and the second reactive impurity into the polar solvent to obtain an IAL salt solution; (c) performing a second scavenging-removal step, comprising: contacting the IAL salt solution with a second scavenger, wherein the second scavenger reacts with the second reactive impurity to form a second scavenger-impurity product; converting the IAL salt into the IAL with a neutralizing agent, wherein the IAL is soluble in a second nonpolar solvent; and separating the IAL from the second scavenger-impurity product, or a derivative thereof formed by the neutralizing agent, by transferring the IAL into the second nonpolar solvent to obtain a purified IAL composition comprising purified IAL.

In some embodiments, the second scavenger-impurity product or derivative thereof is more soluble in a second polar solvent than in the second nonpolar solvent, and the separating further comprises transferring the second scavenger-impurity product or derivative thereof into the second polar solvent. In some embodiments, the neutralizing agent forms a derivative of the second scavenger-impurity product that is a salt of the second scavenger-impurity product that is more soluble in the second polar solvent than in the second nonpolar solvent, and the separating comprises transferring the salt of the second scavenger-impurity product into the second polar solvent. In some embodiments, the second scavenger-impurity product itself is more soluble in the second polar solvent than in the second nonpolar solvent, and the separating further comprises transferring the second scavenger-impurity product into the second polar solvent.

In some embodiments, the present disclosure relates to methods of purifying an ionizable amino lipid (IAL) present in a first composition comprising the IAL, a reactive halide impurity, and a reactive aldehyde impurity in a first nonpolar solvent, comprising: (a) performing a halide scavenging-removal step, comprising: contacting the first composition with a halide scavenger that reacts with the reactive halide impurity to form a scavenger-halide product; and separating the scavenger-halide product from the first composition to obtain a second composition comprising the IAL and reactive aldehyde impurity; (b) performing an acid extraction step, comprising contacting the second composition with an acid that reacts with the IAL to form an IAL salt that is soluble in a polar solvent, and transferring the IAL salt and reactive aldehyde impurity into the polar solvent to obtain an IAL salt solution; (c) performing an aldehyde scavenging-removal step, comprising: contacting the IAL salt solution with an aldehyde scavenger that reacts with the reactive aldehyde impurity to form a scavenger-aldehyde product; converting the IAL salt into the IAL with a neutralizing agent, wherein the IAL is soluble in a second nonpolar solvent; and separating the IAL from the scavenger-aldehyde product, or from a derivative thereof formed by the neutralizing agent, by transferring the IAL into the second nonpolar solvent to obtain a purified IAL composition comprising purified IAL.

In some embodiments, the scavenger-aldehyde product or derivative thereof is more soluble in a second polar solvent than in the second nonpolar solvent, and the separating further comprises transferring the scavenger-aldehyde product or derivative thereof into the second polar solvent. In some embodiments, the neutralizing agent forms a derivative of the scavenger-aldehyde product that is a salt of the scavenger-aldehyde product that is more soluble in the second polar solvent than in the second nonpolar solvent, and the separating comprises transferring the salt of the scavenger-aldehyde product into the second polar solvent. In some embodiments, the scavenger-aldehyde product itself is more soluble in the second polar solvent than in the second nonpolar solvent, and the separating further comprises transferring the scavenger-aldehyde product into the second polar solvent.

In another aspect, the present disclosure relates to methods of purifying an ionizable amino lipid (IAL) using one, two, three, four, or more scavenging-removal steps. For example, in some aspects, the present disclosure relates to methods of purifying an IAL present in a first composition comprising the IAL and a reactive halide impurity in a nonpolar solvent, comprising performing a halide scavenging-removal step, comprising contacting the composition with a scavenger that reacts with the reactive halide impurity to form a scavenger-halide product, and separating the scavenger-halide product from the first composition to obtain a purified IAL composition comprising purified IAL. In some aspects, the present disclosure relates to methods of purifying an ionizable amino lipid (IAL) present in a composition comprising the IAL and a reactive aldehyde impurity in a first nonpolar solvent, comprising performing an acid extraction step, comprising contacting the composition with an acid that reacts with the IAL to form an IAL salt that is soluble in a polar solvent, and transferring the IAL salt and reactive aldehyde impurity into the polar solvent to obtain an IAL salt composition; and performing an aldehyde scavenging-removal step, comprising contacting the IAL salt composition with an aldehyde scavenger that reacts with the reactive aldehyde impurity to form a scavenger-aldehyde product; converting the IAL salt into the IAL with a neutralizing agent, wherein the IAL is soluble in a second nonpolar solvent, and transferring the IAL into the second nonpolar solvent to obtain a purified IAL composition comprising purified IAL. As noted above, in embodiments where the scavenger-aldehyde product or derivative thereof formed by the neutralizing agent is more soluble in a second polar solvent than in the second nonpolar solvent, the separating may further comprise transferring the scavenger-aldehyde product or derivative thereof into the second polar solvent. In some aspects, two or more halide scavenging-removal steps may be used, optionally using different scavengers. In some aspects, two or more aldehyde scavenging-removal steps may be used, optionally using different scavengers. In some aspects, one or more halide scavenging-removal steps may be used in combination with one or more aldehyde scavenging-removal steps.

The various method steps and components discussed above are described in greater detail below. It should be understood that each aspect described below can be used with any other aspect described below, in accordance with the general methodology described above.

### 1. Ionizable Lipids

The methods described herein are useful for purifying ionizable lipids, including ionizable amino lipids (IALs), sometimes referred to in the art as “ionizable cationic lipids.” Thus, in some embodiments, the starting material for a method described herein is a composition comprising one or more ionizable lipids (e.g., IALs) and one or more reactive impurities. The identity of the ionizable lipid (e.g., IAL) to be purified is not particularly limited; the following disclosure of ionizable lipids is provided for illustration only. Ionizable lipids, including IALs, may be used to form lipid delivery agents, such as lipid nanoparticles, which in turn may used to prepare, e.g., mRNA-lipid nanoparticle compositions, such as mRNA vaccines.

As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In some embodiments, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid (“IAL”), sometimes referred to in the art as an “ionizable cationic lipid”. In some embodiments, the ionizable amino lipid may have a positively charged hydrophilic head and a hydrophobic tail that are connected via a linker structure.

In addition to these, an ionizable lipid may also be a lipid including a cyclic amine group.

In some embodiments, the ionizable lipid may be selected from, but not limited to, a ionizable lipid described in International Publication Nos. WO2013/086354 and WO2013/116126.

In some embodiments, the ionizable lipid may be selected from, but not limited to, formulas CLI-CLXXXXII of U.S. Pat. No. 7,404,969.

In some embodiments, the lipid may be a cleavable lipid such as those described in International Publication No. WO2012/170889. In some embodiments, the lipid may be synthesized by methods known in the art and/or as described in International Publication No. WO2013/086354.

In some aspects, the ionizable lipid (e.g., IAL) comprises at least one tertiary amino group, wherein at least one of the three groups of the tertiary amino group comprises a C₆₋₃₀ saturated or unsaturated carbon chain optionally interrupted by an —C(O)O— ester group.

In some aspects, an ionizable lipid (e.g., IAL) according to the present disclosure relates to a compound of Formula (I):

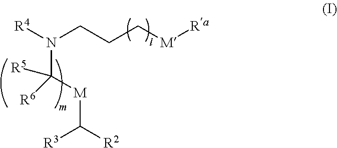

or an isomer thereof, wherein
R′^{a} is R′^{branched}; wherein R′^{branched} is:

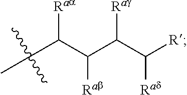

wherein [symbol]
denotes a point of attachment; wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

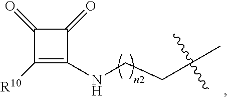

wherein [symbol]
denotes a point of attachment;
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of the compounds of Formula (I), R′^{a} is R′^{branched};
wherein R′^{branched} is

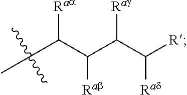

[symbol]

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of the compounds of Formula (I), R′^{a} is R′^{branched}; R′^{branched} is

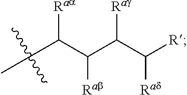

[symbol]

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of the compounds of Formula (I), R′^{a} is R′^{branched}; R′^{branched} is

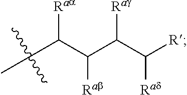

[symbol]

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

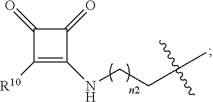

R¹⁰ NH(C₁₋₆ alkyl); n2 is 2; R⁵ is H; each R⁶ is H; M and M′ are each —C(O)O—; R′ is a C₁₋₁₂ alkyl; 1 is 5; and m is 7.

In some embodiments of the compounds of Formula (I), R′^{a} is R′^{branched}; R′^{branched} is

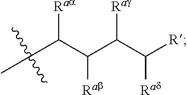

[symbol]

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments, the compound of Formula (I) is selected from:

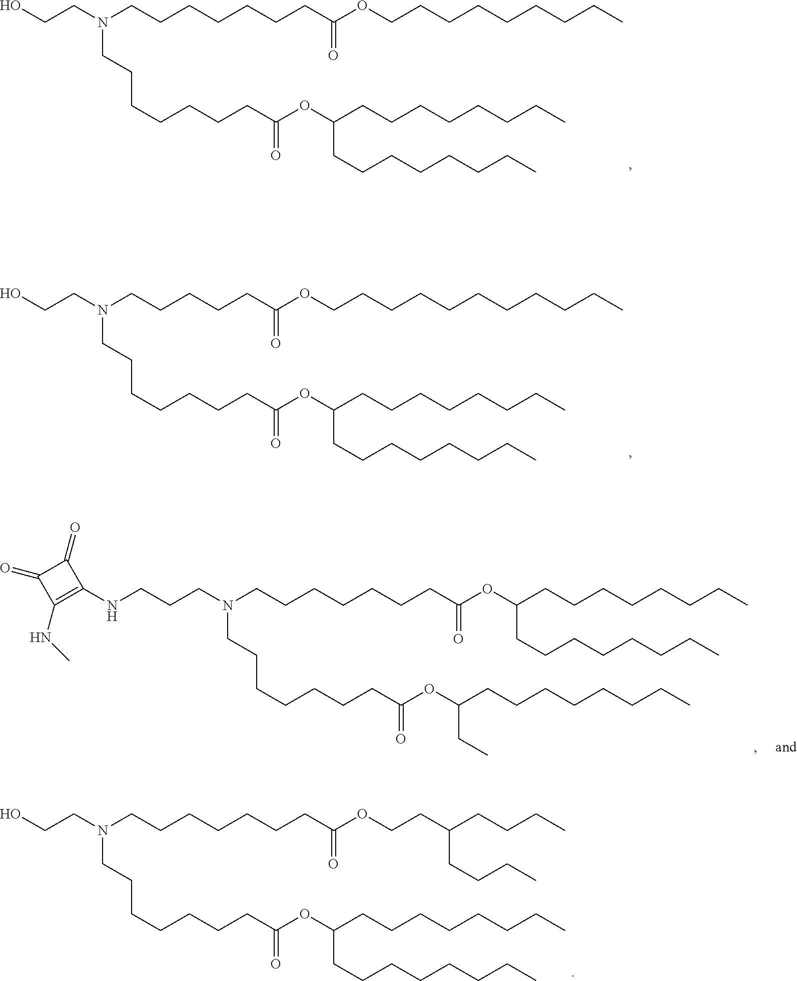 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC]

In some embodiments, the compound of Formula (I) is:

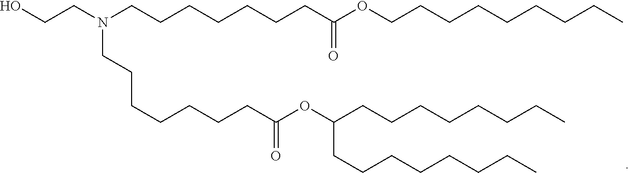 [SMILES: CCCCCCCCCOC(=O)CCCCCCCN(CCO)CCCCCCCC(=O)OC(CCCCCCCC)CCCCCCCC]

In some embodiments, the compound of Formula (I) is:

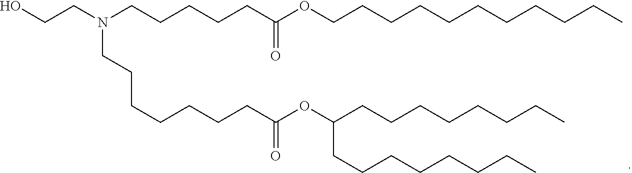 [SMILES: CCCCCCCCCCCOC(=O)CCCCCN(CCO)CCCCCCCC(=O)OC(CCCCCCCC)CCCCCCCC]

In some embodiments, the compound of Formula (I) is:

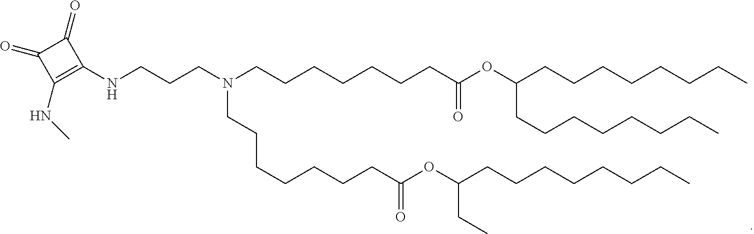 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the compound of Formula (I) is:

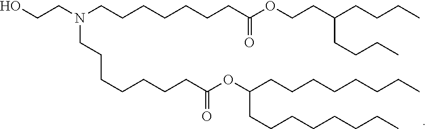 [SMILES: CCCCCCCCC(CCCCCCCC)OC(=O)CCCCCCCN(CCO)CCCCCCCC(=O)OCCC(CCCC)CCCC]

In some aspects, the disclosure relates to a compound of Formula (Ia):

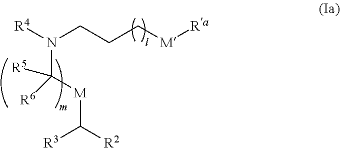

or an isomer thereof, wherein
R^{′}^{^{a}} is R′^{branched}; wherein R′^{branched} is:

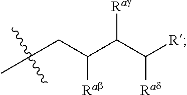

wherein [symbol] denotes a point of attachment; wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

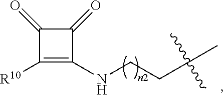

wherein [symbol] denotes a point of attachment;
wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some aspects, the disclosure relates to a compound of Formula (Ib):

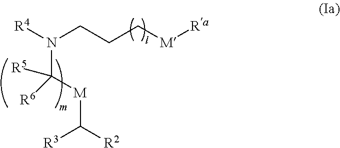

or an isomer thereof, wherein
R′^{a} is R′^{branched}; wherein R′^{branched} is:

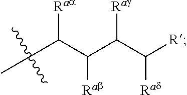

wherein [symbol] denotes a point of attachment; wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of Formula (I) or (Ib), R′^{a} is R′^{branched}; R′^{branched} is

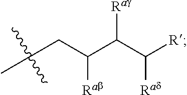

[symbol]

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some embodiments of Formula (I) or (Ib), R′^{a} is R′^{branched}; R′^{branched} is

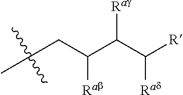

[symbol]

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 3; and m is 7.

In some embodiments of Formula (I) or (Ib), R′^{a} is R′^{branched}; R′^{branched} is

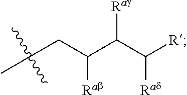

[symbol]

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; 1 is 5; and m is 7.

In some aspects, the disclosure relates to a compound of Formula (Ic):

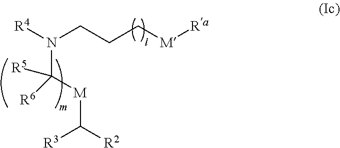

or an isomer thereof, wherein
R′^{a} is R′^{branched}; wherein R′^{branched} is:

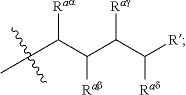

wherein [symbol] denotes a point of attachment; wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is

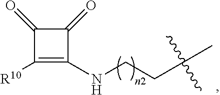

wherein [symbol] denotes a point of attachment;
wherein R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10; each R⁵ is independently selected from the group consisting of C₁₋₃ alkyl, C₂₋₃ alkenyl, and H; each R⁶ is independently selected from the group consisting of C₁₋₃ alkyl,
C₂₋₃ alkenyl, and H; M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, R′^{a} is R′^{branched}; R′^{branched} is

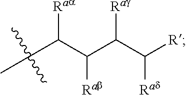

[symbol]

denotes a point of attachment; R^{aβ}, R^{aγ}, and R^{aδ} are each H; R^{aα} is C₂₋₁₂ alkyl; R² and R³ are each C₁₋₁₄ alkyl; R⁴ is

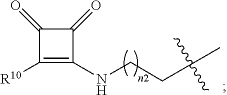

[symbol]

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; 1 is 5; and m is 7.

In some embodiments, the compound of Formula (Ic) is:

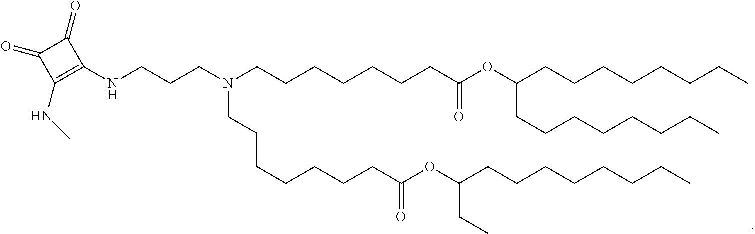 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some aspects, the disclosure relates to a compound of Formula (11):

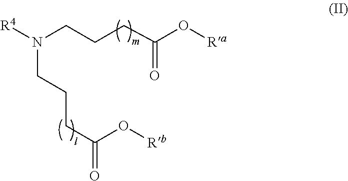

or an isomer thereof, wherein
R′^{a} is R′^{branched} or R′^{cyclic}; wherein R′^{branched} is:

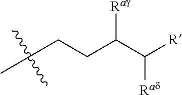

### and R′^{cyclic} is:

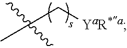

and R′^{b} is:

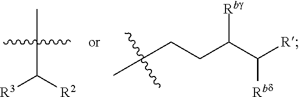

wherein [symbol] denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

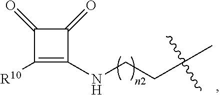

wherein [symbol] denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10; each R′ independently is a C₁₋₁₂ alkyl or C₂₋₁₂ alkenyl; Y^{a} is a C₃₋₆ carbocycle; R*″^{a} is selected from the group consisting of C₁₋₁₅ alkyl and C₂₋₁₅ alkenyl; and s is 2 or 3; m is selected from 1, 2, 3, 4, 5, 6, 7, 8, and 9; and 1 is selected from 1, 2, 3, 4, 5, 6, 7, 8, and 9.

In some aspects, the disclosure relates to a compound of Formula (II-a):

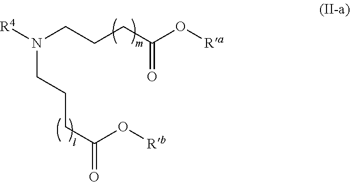

or an isomer thereof, wherein
R′^{a} is R′^{branched} or R′^{cyclic}; wherein R′^{branched} is:

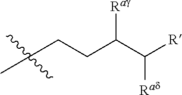

### and R′^{b} is:

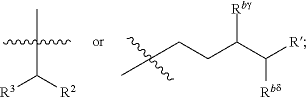

wherein [symbol] denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

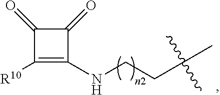

wherein [symbol] denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10; each R′ independently is a C₁₋₁₂ alkyl or C₂₋₁₂ alkenyl; m is selected from 1, 2, 3, 4, 5, 6, 7, 8, and 9; and 1 is selected from 1, 2, 3, 4, 5, 6, 7, 8, and 9.

In some aspects, the disclosure relates to a compound of Formula (II-b):

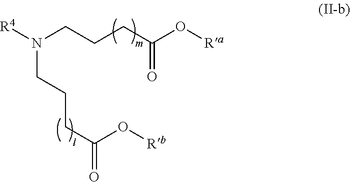

or an isomer thereof, wherein
R′^{a} is R′^{branched} or R′^{cyclic}; wherein R′^{branched} is:

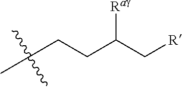

### and R′^{b} is:

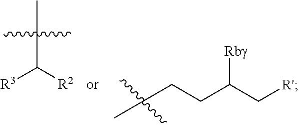

wherein [symbol] denotes a point of attachment; R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

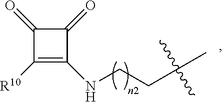

wherein [symbol] denotes a point of attachment;
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some aspects, the disclosure relates to a compound of Formula (II-c):

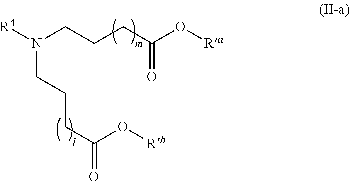

or an isomer thereof, wherein
R′^{a} is R′^{branched} or R′^{cyclic}; wherein R′^{branched} is:

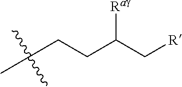

### and R′^{b} is:

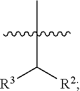

wherein [symbol]
denotes a point of attachment; wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

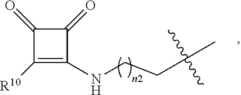

wherein [symbol] denotes a point of attachment;
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some aspects, the disclosure relates to a compound of Formula (II-d):

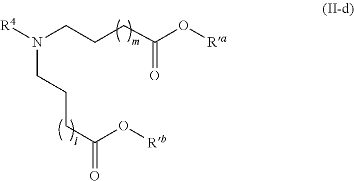

or an isomer thereof, wherein
R′^{a} is R′^{branched} or R′^{cyclic}; wherein R′^{branched} is:

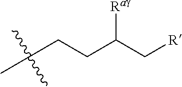

### and R′^{b} is:

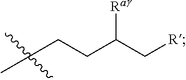

wherein [symbol] denotes a point of attachment; wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

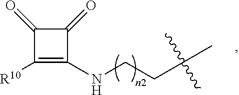

wherein [symbol] denotes a point of attachment;
R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some aspects, the disclosure relates to a compound of Formula (II-e):

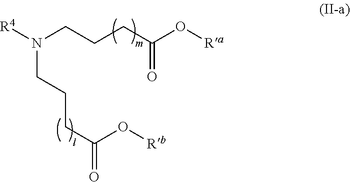

or an isomer thereof, wherein
R′^{a} is R′^{branched} or R′^{cyclic}; wherein R′^{branched} is:

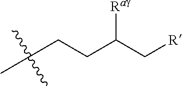

### and R′^{b} is:

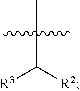

wherein [symbol] denotes a point of attachment; wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9; and
l is selected from 1, 2, 3, 4, 5, 6, 7, 8, and 9.

In some embodiments of the compound of Formula (II), (II-a), (II-b), (II-c), (II-d), or (II-e), m and 1 are each independently selected from 4, 5, and 6. In some embodiments of the compound of Formula (II), (II-a), (II-b), (II-c), (II-d), or (II-e), m and 1 are each 5.

In some embodiments of the compound of Formula (II), (II-a), (II-b), (II-c), (II-d), or (II-e), each R′ independently is a C₁₋₁₂ alkyl. In some embodiments of the compound of Formula (II), (II-a), (II-b), (II-c), (II-d), or (II-e), each R′ independently is a C₂₋₅ alkyl.

In some embodiments of the compound of Formula (II), (II-a), (II-b), (II-c), (II-d), or (II-e), R′^{b} is:

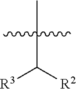

and R² and R³ are each independently a C₁₋₁₄ alkyl. In some embodiments of the compound of Formula (II), (II-a), (II-b), (II-c), (II-d), or (II-e), R′^{b} is:

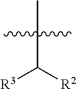

and R² and R³ are each independently a C₆₋₁₀ alkyl.

In some embodiments of the compound of Formula (II), (II-a), (II-b), (II-c), (II-d), or (II-e), R′^{b} is:

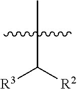

and R² and R³ are each a C₈ alkyl.

In some embodiments of the compound of Formula (II), (II-a), (II-b), (II-c), (II-d), or (II-e), R′^{branched} is:

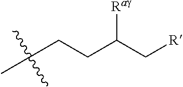

### and R′^{b} is:

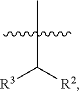

R^{aγ} is a C₁₋₂ alkyl and R² and R³ are each independently a C₆₋₁₀ alkyl.

In some embodiments of the compound of Formula (II), (II-a), (II-b), (II-c), (II-d), or (II-e), R′^{branched} is:

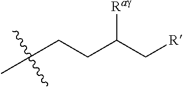

### and R′^{b} is:

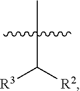

R^{aγ} is a C₂₋₆ alkyl and R² and R³ are each independently a C₆₋₁₀ alkyl.

In some embodiments of the compound of Formula (II), (II-a), (II-b), (II-c), (II-d), or (II-e), R′^{branched} is:

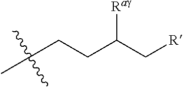

### R′^{b} is:

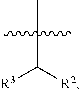

R^{aγ} is a C₂₋₆ alkyl, and R² and R³ are each a C₈ alkyl.

In some embodiments of the compound of Formula (II), (II-a), (II-b), (II-c), (II-d), or (II-e), R′^{branched} is:

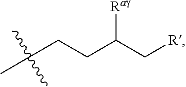

### R′^{b} is:

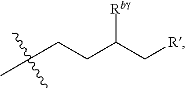

and R^{aγ} and R^{bγ} are each a C₁₋₁₂ alkyl.

In some embodiments of the compound of Formula (II), (II-a), (II-b), (II-c), (II-d), or (II-e), R′^{branched} is:

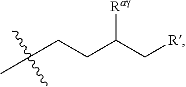

### R′^{b} is:

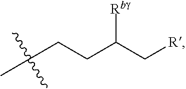

and R^{aγ} and R^{bγ} are each a C₂₋₆ alkyl.

In some embodiments of the compound of Formula (II), (II-a), (II-b), (II-c), (II-d), or (II-e), m and l are each independently selected from 4, 5, and 6 and each R′ independently is a C₁₋₁₂ alkyl.

In some embodiments of the compound of Formula (II), (II-a), (II-b), (II-c), (II-d), or (II-e), m and 1 are each 5 and each R′ independently is a C₂₋₅ alkyl.

In some embodiments of the compound of Formula (II), (II-a), (II-b), (II-c), (II-d), or (II-e), R′^{branched} is:

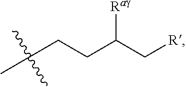

### R′^{b} is:

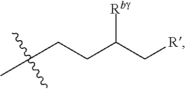

m and l are each independently selected from 4, 5, and 6, each R′ independently is a C₁₋₁₂ alkyl, and R^{aγ} and R^{bγ} are each a C₁₋₁₂ alkyl.

In some embodiments of the compound of Formula (II), (II-a), (II-b), (II-c), (II-d), or (II-e), R′^{branched} is:

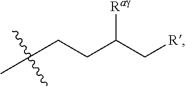

### R′^{b} is:

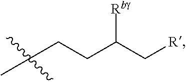

m and l are each 5, each R′ independently is a C₂₋₅ alkyl, and R^{aγ} and R^{bγ} are each a C₂₋₆ alkyl.

In some embodiments of the compound of Formula (II), (II-a), (II-b), (II-c), (II-d), or (II-e), R′^{branched} is:

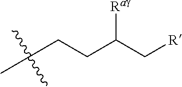

### and R′^{b} is:

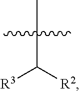

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl.

In some embodiments of the compound of Formula (II), (II-a), (II-b), (II-c), (II-d), or (II-e), R′^{branched} is:

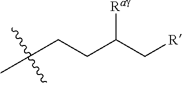

### and R′^{b} is:

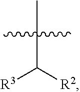

m and l are each 5, R′ is a C₂₋₅ alkyl, R^{aγ} is a C₂₋₆ alkyl, and R² and R³ are each a C₈ alkyl.

In some embodiments of the compound of Formula (II), (II-a), (II-b), (II-c), (II-d), or (II-e), R⁴ is

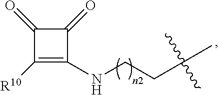

wherein R¹⁰ is NH(C₁₋₆ alkyl) and n2 is 2.

In some embodiments of the compound of Formula (II), (II-a), (II-b), (II-c), (II-d), or (II-e), R⁴ is

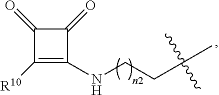

wherein R¹⁰ is NH(CH₃) and n2 is 2.

In some embodiments of the compound of Formula (II), (II-a), (II-b), (II-c), (II-d), or (II-e), R′^{branched} is:

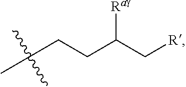

### R′^{b} is:

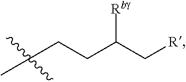

m and l are each independently selected from 4, 5, and 6, each R′ independently is a C₁₋₁₂ alkyl, R^{aγ} and R^{bγ} are each a C₁₋₁₂ alkyl, and R⁴ is

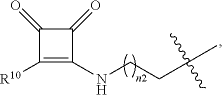

wherein R¹⁰ is NH(C₁₋₆ alkyl), and n2 is 2.

In some embodiments of the compound of Formula (II), (II-a), (II-b), (II-c), (II-d), or (II-e), R′^{branched} is:

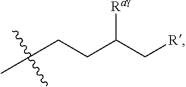

### R′^{b} is:

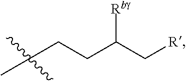

m and l are each 5, each R′ independently is a C₂₋₅ alkyl, R^{aγ} and R^{bγ} are each a C₂₋₆ alkyl, and R⁴ is

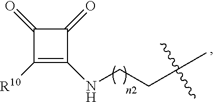

wherein R¹⁰ is NH(CH₃) and n2 is 2.

In some embodiments of the compound of Formula (II), (II-a), (II-b), (II-c), (II-d), or (II-e), R′^{branched} is:

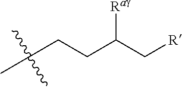

### and R′^{b} is:

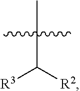

m and l are each independently selected from 4, 5, and 6, R′ is a C₁₋₁₂ alkyl, R² and R³ are each independently a C₆₋₁₀ alkyl, R^{aγ} is a C₁₋₂ alkyl, and R⁴ is

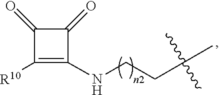

wherein R¹⁰ is NH(C₁₋₆ alkyl) and n2 is 2.

In some embodiments of the compound of Formula (II), (II-a), (II-b), (II-c), (II-d), or (II-e), R′^{branched} is:

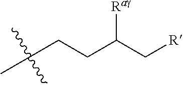

### and R′^{b} is:

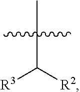

m and l are each 5, R′ is a C₂₋₅ alkyl, R^{aγ} is a C₂₋₆ alkyl, R² and R³ are each a C₈ alkyl, and R⁴ is

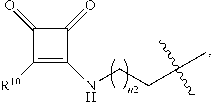

wherein R¹⁰ is NH(CH₃) and n2 is 2.

In some embodiments of the compound of Formula (II), (II-a), (II-b), (II-c), (II-d), or (II-e), R⁴ is —(CH₂)ₙOH and n is 2, 3, or 4. In some embodiments of the compound of Formula (II), (II-a), (II-b), (II-c), (II-d), or (II-e), R⁴ is —(CH₂)ₙOH and n is 2.

In some embodiments of the compound of Formula (II), (II-a), (II-b), (II-c), (II-d), or (II-e), R′^{branched} is:

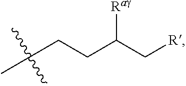

### R′^{b} is:

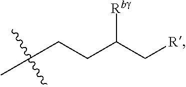

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4.

In some embodiments of the compound of Formula (II), (II-a), (II-b), (II-c), (II-d), or (II-e), R′^{branched} is:

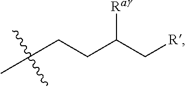

### R′^{b} is:

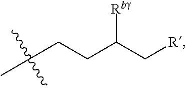

m and l are each 5, each R′ independently is a C₂₋₅ alkyl, R^{aγ} and R^{bγ} are each a C₂₋₆ alkyl, R⁴ is —(CH₂)ₙOH, and n is 2.

In some aspects, the disclosure relates to a compound of Formula (II-f):

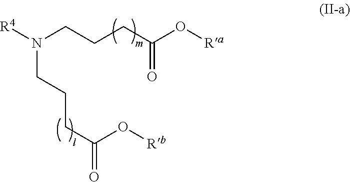

or an isomer thereof, wherein
R′^{a} is R′^{branched} or R′^{cyclic}; wherein R′^{branched} is:

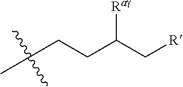

### and R′^{b} is:

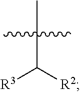

wherein [symbol]
denotes a point of attachment; R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (II-f), m and l are each 5, and n is 2, 3, or 4.

In some embodiments of the compound of Formula (II-f) R′ is a C₂₋₅ alkyl, R^{aγ} is a C₂₋₆ alkyl, and R² and R³ are each a C₆₋₁₀ alkyl.

In some embodiments of the compound of Formula (II-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some aspects, the disclosure relates to a compound of Formula (II-g):

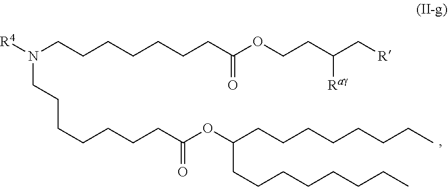

wherein R^{aγ} is a C₂₋₆ alkyl; R′ is a C₂₋₅ alkyl; and R⁴ is selected from the group consisting of —(CH₂)ₙOH wherein n is selected from the group consisting of 3, 4, and 5, and

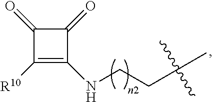

wherein [symbol] denotes a point of attachment, R¹⁰ is NH(C₁₋₆ alkyl), and n2 is selected from the group consisting of 1, 2, and 3.

In some aspects, the disclosure relates to a compound of Formula (II-h):

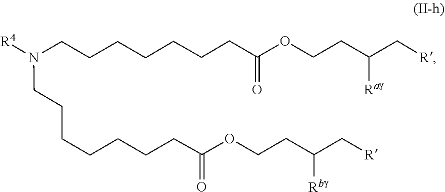

wherein R^{aγ} and R^{bγ} are each independently a C₂₋₆ alkyl; each R′ independently is a C₂₋₅ alkyl; and R⁴ is selected from the group consisting of —(CH₂)ₙOH wherein n is selected from the group consisting of 3, 4, and 5, and

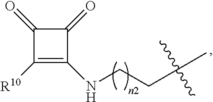

wherein [symbol] denotes a point of attachment, R¹⁰ is NH(C₁₋₆ alkyl), and n2 is selected from the group consisting of 1, 2, and 3.

In some embodiments of the compound of Formula (II-g) or (II-h), R⁴ is

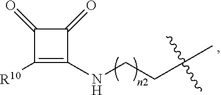

wherein R¹⁰ is NH(CH₃) and n2 is 2.

In some embodiments of the compound of Formula (II-g) or (II-h), R⁴ is —(CH₂)₂OH.

In some aspects, the disclosure relates to a compound having the Formula (III):

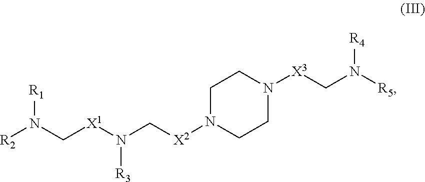

or an isomer thereof, wherein R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
each M is independently selected from the group consisting
of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
X¹, X², and X³ are independently selected from the group consisting of a bond, —CH₂—, —(CH₂)₂—, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—; each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H; and
each R″ is independently selected from the group consisting of C₃₋₁₂ alkyl and C₃₋₁₂ alkenyl, and wherein:
i) at least one of X¹, X², and X³ is not —CH₂—; and/or
ii) at least one of R₁, R₂, R₃, R₄, and R₅ is —R″MR′.

In some embodiments, R₁, R₂, R₃, R₄, and R₅ are each C₅₋₂₀ alkyl; X¹ is —CH₂—; and X² and X³ are each —C(O)—.

In some embodiments, the compound of Formula (III) is:

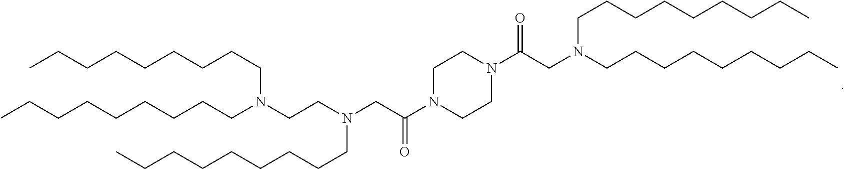 [SMILES: CCCCCCCCCN(CCCCCCCCC)CCN(CCCCCCCCC)CC(=O)N1CCN(C(=O)CN(CCCCCCCCC)CCCCCCCCC)CC1]

In some embodiments, the ionizable lipids (e.g., IALs) are one or more of the compounds described in WO2020/146805; WO2020/081938; WO2020/214946; WO2019/036030; WO2019/036000; WO2019/036028; WO2019/036008; WO2018/200943; WO2018/191657; WO2017/117528; WO2017/075531; WO2017/004143; WO2015/199952; and WO2015/074085.

In some embodiments, the ionizable lipids (e.g., IALs) are one or more of the compounds described in WO2020/146805 having structure:

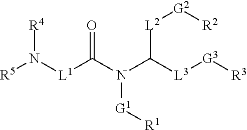

or a tautomer, or stereoisomer thereof, wherein:
R¹ is optionally substituted C₁-C₂₄ alkyl or optionally substituted C₂-C₂₄ alkenyl;
R² and R³ are each independently optionally substituted C₁-C₃₆ alkyl;
R⁴ and R⁵ are each independently optionally substituted C₁-C₆ alkyl, or R⁴ and R⁵ join, along with the N to which they are attached, to form a heterocyclyl or heteroaryl;
L¹, L², and L³ are each independently optionally substituted C₁-C₁₈ alkylene;
G¹ is a direct bond, —(CH₂)ₙO(C═O)—, —(CH₂)ₙ(C═O)O—, or —(C═)—;
G² and G³ are each independently —(C═O)O— or —O(C═O)—; and n is an integer greater than 0.

In some embodiments, the ionizable lipids (e.g., IALs) are one or more of the compounds described in WO2020/081938 having structure:

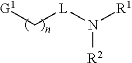

or a tautomer or stereoisomer thereof, wherein:
G¹ is —N(R³)R⁴ or —OR⁵;
R¹ is optionally substituted branched, saturated or unsaturated C₁₂-C₃₆ alkyl;
R² is optionally substituted branched or unbranched, saturated or unsaturated C₁₂-C₃₆ alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;
R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;
R⁵ is H or optionally substituted C₁-C₆ alkyl;
L is —C(═O)—, C₆-C₁₂ alkylene, C₆-C₁₂ alkenylene, or C₂-C₁₂alkynylene (e.g., C₂-C₆ alkynylene); and
n is an integer from 1 to 12.

In some embodiments, the ionizable lipids (e.g., IALs) are one or more of the compounds described in WO2020/214946 having structure:

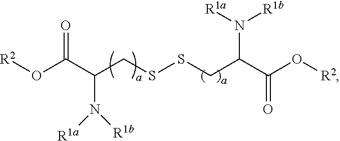

wherein each R^{1a} is independently hydrogen, R^{1c}, or R^{3d}; each Rib is independently R^{1c} or R^{1d}; each R^{1c} is Independently —(CH)₂C(O)X¹R³; each R^{1d} is independently —C(O)R⁴; each R² is independently —[C(R^{2a})₂]_{c}; R^{2b}; each R^{2a} is independently hydrogen or lower alkyl (e.g., C₁-C₆alkyl); R^{2b} is —N(L₁-B)₂, —(OCH₂CH₂)₆OH; or —(OCH₂CH₂)₆OCH₃; each R³ and R⁴ is independently aliphatic (e.g., C₆-C₃₀ aliphatic); each L₁ is independently alkylene (e.g., C₁-C₁₀ alkylene); each B is independently hydrogen or an ionizable nitrogen-containing group; each X¹ is independently a covalent bond or O; each a is independently an integer (e.g., 1-10); each b is independently an integer (e.g., 1-10); and each c is independently an integer (e.g., 1-10).

In some embodiments, the ionizable lipids (e.g., IALs) are one or more of the compounds described in WO2019/036030 having the structure:

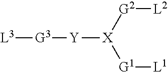

or a prodrug or stereoisomer thereof, wherein:
X is N, and Y is absent; or X is CR, and Y is NR;
L¹ is —O(C═O)R¹, —(C═O)OR¹, —C(═O)R¹, —OR¹, S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²
L³ is —O(C═O)R³ or —(C═O)OR³;
G¹ and G² are each independently C₂-C₁₂ alkylene or C₂-C₁₂ alkenylene;
G³ is C₁-C₂₄ alkylene, C₂-C₂₄ alkenylene, C₁-C₂₄ heteroalkylene or C₂-C₂₄ heteroalkenylene when X is CR, and Y is NR; and G³ is C₁-C₂₄ heteroalkylene or C₂-C₂₄ heteroalkenylene when X is N, and Y is absent;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
each R is independently H or C₁-C₁₂ alkyl;
R¹, R² and R³ are each independently C₁-C₂₄ alkyl or C₂-C₂₄ alkenyl; and
x is 0, 1 or 2, and
wherein each alkyl, alkenyl, alkylene, alkenylene, heteroalkylene and heteroalkenylene is independently substituted or unsubstituted unless otherwise specified.

In some embodiments, the ionizable lipids (e.g., IALs) are one or more of the compounds described in WO2019/036000 having the structure:

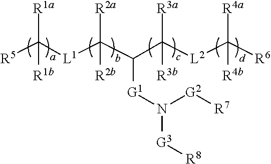

or a tautomer, prodrug, or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C₁-C₂ alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) Ria is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is H or C₁-C₂₀ alkyl;
R⁸ is OH, —N(R⁹)(C═O)R¹⁰, —(C═O)NR⁹R¹⁰, —NR⁹R¹⁰, —(C═O)OR¹¹ or —O(C═O)R¹¹, provided that G³ is C₄-C₆ alkylene when R⁸ is —NR⁹R¹⁰,
R⁹ and R¹⁰ are each independently H or C₁-C₁₂ alkyl;
R¹¹ is aralkyl;
a, b, c and d are each independently an integer from I to 24; and x is 0, 1 or 2,
wherein each alkyl, alkylene and aralkyl is optionally substituted.

In some embodiments, the ionizable lipids (e.g., IALs) are one or more of the compounds described in WO2019/036028 having the structure:

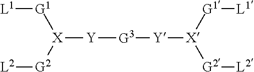

or a prodrug or stereoisomer thereof,
wherein:
X and X′ are each independently N or CR;
Y and Y′ are each independently absent, —O(C═O)—, —(C═O)O— or NR, provided that:
a) Y is absent when X is N;
b) Y′ is absent when X′ is N;
c) Y is —O(C═O)—, —(C═O)O— or NR when X is CR; and
d) Y′ is —O(C═O)—, —(C═O)O— or NR when X′ is CR,
L¹ and L^{1′} are each independently —O(C═O)R¹, —(C═O)OR¹, —C(═O)R¹, —OR¹, —S(O)_{z}R¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² and L² are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹, G^{1′}, G² and G^{2′} are each independently C₂-C₁₂ alkylene or C₂-C₁₂ alkenylene;
G³ is C₂-C₂₄ heteroalkylene or C₂-C₂₄ heteroalkenylene;
R^{a}, R^{b}, R^{d} and R^{e} are, at each occurrence, independently H, C₁-C₁₂ alkyl or C₂-C₁₂ alkenyl;
R^{c} and R^{f} are, at each occurrence, independently C₁-C₁₂ alkyl or C₂-C₁₂ alkenyl;
R is, at each occurrence, independently H or C₁-C₁₂ alkyl;
R¹ and R² are, at each occurrence, independently branched C₆-C₂₄ alkyl or branched C₆-C₂₄ alkenyl;
z is 0, 1 or 2, and
wherein each alkyl, alkenyl, alkylene, alkenylene, heteroalkylene and heteroalkenylene is independently substituted or unsubstituted unless otherwise specified.

In some embodiments, the ionizable lipids (e.g., IALs) are one or more of the compounds described in WO2019/036008 have the structure:

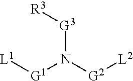

or a prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f} or —NR^{d}C(═O)OR² or a direct bond to R²;
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴)R⁵;
R⁴ is C₁-C₁₂ alkyl;
R⁵ is substituted C₁-C₁₂ alkyl;
and x is 0, 1 or 2, and
wherein each alkyl, alkenyl, alkylene, alkenylene, cycloalkylene, cycloalkenylene, aryl and aralkyl is independently substituted or unsubstituted unless otherwise specified.

In some embodiments, the ionizable lipids (e.g., IALs) are one or more of the compounds described in WO 2018/200943 having the structure:

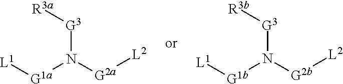

or a prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G^{1a} and G^{2a} are each independently C₂-C₁₂ alkylene or C₂-C₁₂ alkenylene;
G^{1b} and G^{2b} are each independently C₁-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₂-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R^{3a} is —C(═O)N(R^{4a})R^{5a} or —C(═O)OR⁶;
R^{3b} is —NR^{4b} C(═O)R^{5b}.
R^{4a} is C₁-C₁₂ alkyl;
R^{4b} is H, C₁-C₁₂ alkyl or C₂-C₁₂ alkenyl;
R^{5a} is H, C₁-C₈ alkyl or C₂-C₈ alkenyl;
R^{5b} is C₂-C₁₂ alkyl or C₂-C₁₂ alkenyl when R^{4b} is H; or R^{5b} is C₁-C₁₂ alkyl or C₂-C₁₂ alkenyl when R^{4b} is C₁-C₁₂ alkyl or C₂-C₁₂ alkenyl;
R⁶ is H, aryl or aralkyl; and
x is 0, 1 or 2, and
wherein each alkyl, alkenyl, alkylene, alkenylene, cycloalkylene, cycloalkenylene, aryl, and aralkyl is independently substituted or unsubstituted.

In some embodiments, the ionizable lipids (e.g., IALs) are one or more of the compounds described in WO2018/191657 having the structure

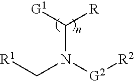

or a prodrug or stereoisomer thereof, wherein:
G¹ is —OH, —NR³R⁴, —(C═O)NR⁵ or —NR³(C═O)R⁵;
G² is —CH₂— or —(C═O)—;
R is, at each occurrence, independently H or OH;
R¹ and R² are each independently optionally substituted branched, saturated or unsaturated C₁₂-C₃₆ alkyl;
R³ and R⁴ are each independently H or optionally substituted straight or branched, saturated or unsaturated C₁-C₆ alkyl;
R⁵ is optionally substituted straight or branched, saturated or unsaturated C₁-C₆ alkyl; and
n is an integer from 2 to 6.

In some embodiments, the ionizable lipids (e.g., IALs) are one or more of the compounds described in WO2017/117528 having the structure:

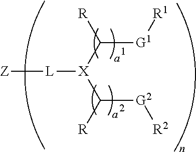

or a prodrug or stereoisomer thereof, wherein:
one of G¹ or G² is, at each occurrence, —O(C═O)—, —(C═O)O—, —C(═O)—, —O—, —S(O)_{y}, —S—S—, —C(═O)S—, SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O—, and the other of G¹ or G² is, at each occurrence, —O(C═O)—, —(C═O)O—C(═O)—, —O—, —S(O)_{y}, —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O— or a direct bond;
L is, at each occurrence, ˜O(C═O)—, wherein ˜ represents a covalent bond to X;
X is CR^{a};
Z is alkyl, cycloalkyl or a monovalent moiety comprising at least one polar functional group when n is 1; or Z is alkylene, cycloalkylene or a polyvalent moiety comprising at least one polar functional group when n is greater than 1;
R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;
R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;
R¹ and R² have, at each occurrence, the following structure, respectively:

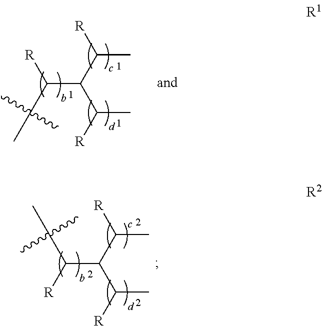

wherein:
a¹ and a² are, at each occurrence, independently an integer from 3 to 12;
b¹ and b² are, at each occurrence, independently 0 or 1;
c¹ and c² are, at each occurrence, independently an integer from 5 to 10;
d¹ and d² are, at each occurrence, independently an integer from 5 to 10;
y is, at each occurrence, independently an integer from 0 to 2; and
n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

In some embodiments, the ionizable lipids (e.g., IALs) are one or more of the compounds described in WO 2017/075531 having the structure:

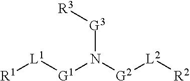

or a prodrug or stereoisomer thereof, wherein:
one of L¹ or L² is —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} a is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —NR⁵C(═O)R⁴;
R⁴ is C₁-C₁₂ alkyl;
R⁵ is H or C₁-C₆ alkyl; and
x is 0, 1 or 2.

In some embodiments, the ionizable lipids (e.g., IALs) are one or more of the compounds described in WO 2017/004143 having the structure:

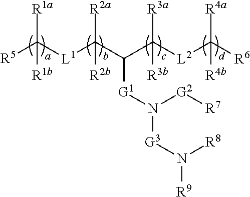

or a tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C₁-C₂ alkylene, —(C═O)—, —O(C═O)—, —SC(═O)—, —NR^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond; R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and
x is 0, 1 or 2.

In some embodiments, the ionizable lipids (e.g., IALs) are one or more of the compounds described in WO 2015/199952 having the structure:

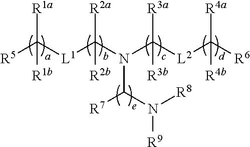

or a tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond; R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl;
R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24;
b and c are each independently an integer from 1 to 24; and
e is 1 or 2,
provided that:
at least one of R^{1a}, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

In some embodiments, the ionizable lipids (e.g., IALs) are one or more of the compounds described in WO2015/074085 having the structure:

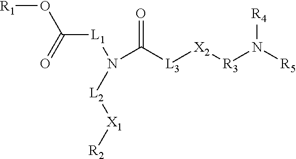

wherein:
R₁ and R₂ are the same or different, each a linear or branched alkyl with 1-9 carbons, or an alkenyl or alkynyl with 2 to 11 carbon atoms,
L₁ and L₂ are the same or different, each a linear alkyl having 5 to 18 carbon atoms, or form a heterocycle with N,
X₁ is a bond, or is —CO—O— whereby L₂-CO—O—R₂ is formed
X₂ is S or O,
L₃ is a bond or a lower alkyl, or form a heterocycle with N,
R³ is a lower alkyl, and
R⁴ and R₅ are the same or different, each a lower alkyl;
or an isomer thereof.

In some embodiments, the ionizable lipids (e.g., IALs) are one or more of the compounds described in Buschmann, M. D. et al., Vaccines, 2021, 9, 65, (the structures provided below include their theoretical pKas):

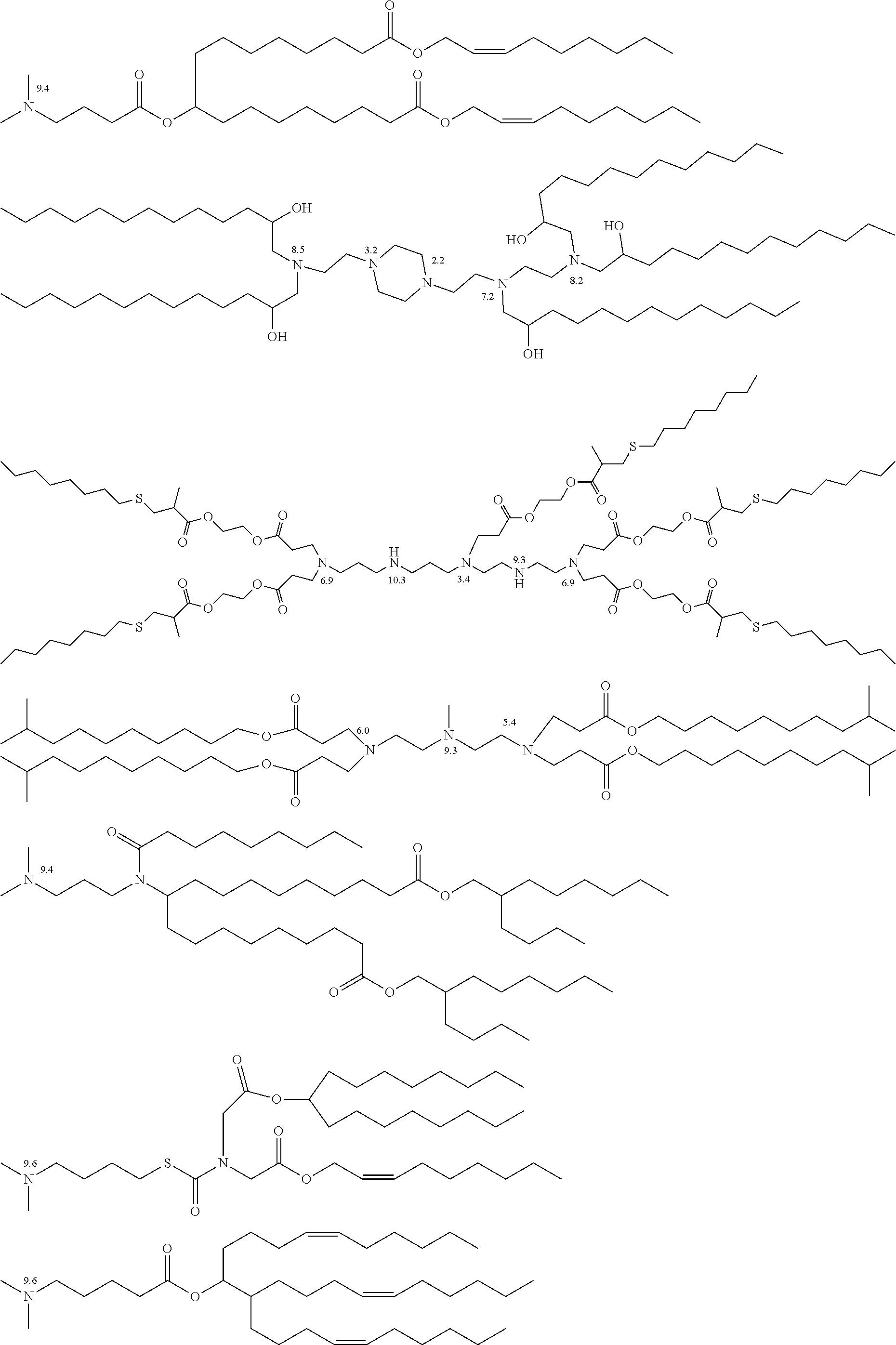

In some embodiments, the ionizable lipid (e.g., IAL) is selected from:

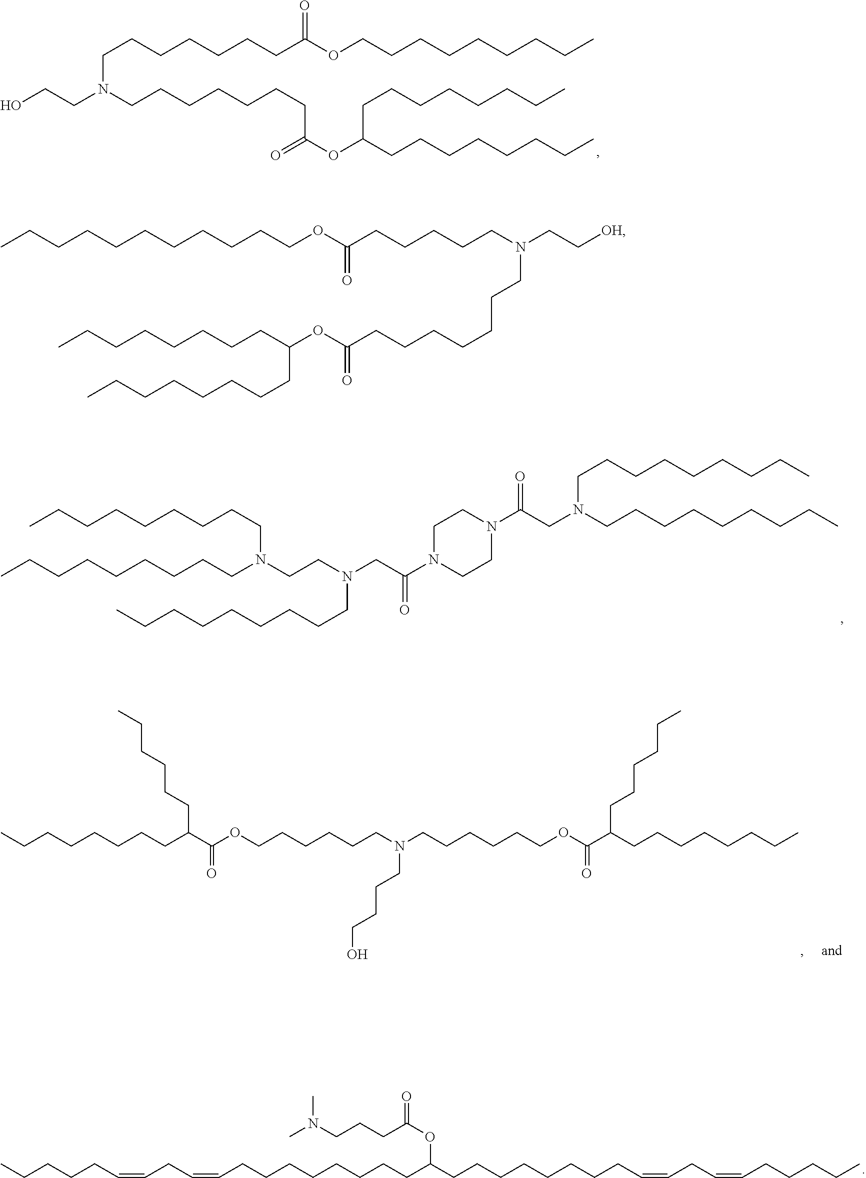

### 2. Reactive Impurities

Reactive impurities are electrophilic molecules that can react with nucleophilic moieties on polynucleotides such as mRNA, and subsequently affect the purity and potency of a product. For example, reactive aldehyde impurities may react with polynucleotides such as mRNA to form adducts, which have been shown to have reduced translation competency. Reactive impurities that may be present in IAL compositions include alkyl halides and carbonyls, such as aldehydes and ketones. The reactive impurities may be residual reagents used in the synthesis of IALs or side products produced during the synthesis of IALs.

Even when reactive impurities are present in IAL compositions at concentrations below detection limits for typical characterization methods used to determine IAL purity (e.g., UPLC-CAD), their presence may be sufficient to undermine the purity and/or potency of a product made using the IAL, such as an LNP product, including a product comprising an LNP and polynucleotide, such as mRNA. Without being bound by theory, it is believed this is because, despite being present at low concentrations, the reactive impurities may react with nucleophilic moieties on polynucleotide molecules, such as mRNA, to form adducts. Adduct formation, even at low levels, may be associated with significant reductions in activity, such as reduced translation of mRNA. For example, in the context of LNP compositions encapsulating mRNA, the presence of reactive impurities may be associated with a reduced content of translation competent mRNA. Thus, methods of removing reactive impurities from IAL compositions as described herein permit the preparation of LNP-polynucleotide compositions (e.g., LNP mRNA compositions) with higher therapeutic potential.

The amount of adduct impurity (e.g., aldehyde-polynucleotide adduct impurity or alkyl halide-polynucleotide adduct impurity) present in a composition may be measured by reverse phase ion pair chromatography (RP-IP HPLC), such as described in U.S. application Ser. No. 17/508,786 and Packer et al., Nature Comm. (2021) 12: 6777.

### (a) Halides

In some embodiments, the reactive impurities comprise one or more reactive halides, such as reactive alkyl halides. The identity of reactive halides that can be removed from IAL compositions by methods described herein is not particularly limited; the following disclosure is provided for illustration only.

In some embodiments, the reactive halide comprises one or more alkyl halide compounds according to Formula (B):

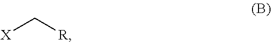

wherein X is F, Cl, Br, I, or any combination thereof, and R is a substituted or unsubstituted branched or unbranched alkyl, alkenyl, or alkynyl group. By way of non-limiting example, the alkyl halide may comprise Compound B below:

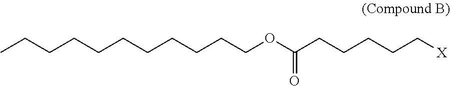

wherein X is F, Cl, Br, or I. For ease of reference, the iodide form of Compound B is referred to herein as “Compound B-iodide,” the bromide form of Compound B is referred to herein as “Compound B-bromide,” and so forth.

### (b) Carbonyls

In some embodiments, the reactive impurities comprise one or more carbonyl compounds (e.g., aldehydes or ketones). In some embodiments, the reactive impurities comprise one or more aldehydes. In some embodiments, the reactive impurities comprise one or more ketones. In some embodiments, the reactive impurities comprise one or more aldehydes and one or more ketones. The identity of reactive carbonyls that can be removed from IAL compositions by methods described herein is not particularly limited; the following disclosure is provided for illustration only.

In some embodiments, the reactive impurities comprise a reactive aldehyde comprising one or more compounds according to Formula (C):

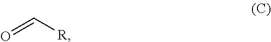

wherein R is a substituted or unsubstituted branched or unbranched alkyl, alkenyl, or alkynyl group. By way of non-limiting example, the one or more aldehyde impurities may comprise Compound C (decanal) below:

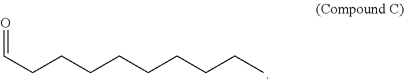

### 3. Halide Scavenging-Removal

In some embodiments, reactive halide impurities, such as alkyl halides, are removed from a first composition comprising the IAL and the reactive alkyl halide impurities by contacting the first composition with a scavenger, such as an alkyl halide scavenger. For purposes of this disclosure, an “alkyl halide scavenger” is a compound that selectively reacts with an alkyl halide impurity (over the IAL) to produce a product that has different solubility from the IAL. For instance, the alkyl halide scavenger may react with an alkyl halide soluble in a nonpolar solvent (e.g., heptane) to produce a product (a scavenger-halide product) that is soluble in a polar solvent (e.g., acetonitrile) and one or more inorganic salts.

Non-limiting examples of suitable alkyl halide scavengers are shown in Scheme 1 below, along with representative reaction products with reactive alkyl halides. In some embodiments, the alkyl halide scavenger may be one or more classes of compounds selected from phosphines, amines, thiols, and sulfoxides. In some embodiments the alkyl halide scavenger comprises one or more moieties selected from cyclic primary amines, acyclic primary amines, secondary amines, and tertiary amines. In some embodiments, the alkyl halide scavenger may comprise or consist of one or more amines selected from trimethylamine, 1,4-diazabicyclo[2.2.2]octane (also known as “DABCO” or triethylenediamine), pyridine, and 4-dimethylaminopyride (“DMAP”). In some embodiments, the alkyl halide scavenger may comprise or consist of one or more phosphines selected from triphenylphosphine and tributylphosphine. In some embodiments, the alkyl halide scavenger may comprise or consist of one or more thiols selected from alkylthiols (e.g., dodecanethiol, undecanethiol, decanethiol, nonanethiol, octanethiol, hexanethiol, etc.). In some embodiments, the alkyl halide scavenger may comprise or consist of one or more sulfoxides, such as dimethylsulfoxide.

In some embodiments, the alkyl halide scavenger comprises or consists of one or more compounds selected from triphenylphosphine, tributylphosphine, triethylamine, triethylenediamine (DABCO), 4-dimethylaminopyridine (DMAP), dimethylsulfoxide, and dodecanethiol. In some embodiments, the halide scavenger comprises or consists of one or more compounds selected from tributylphosphine, triethylamine, triethylenediamine (DABCO), and 4-dimethylaminopyridine (DMAP). In some embodiments, the halide scavenger comprises or consists of triethylenediamine (DABCO).

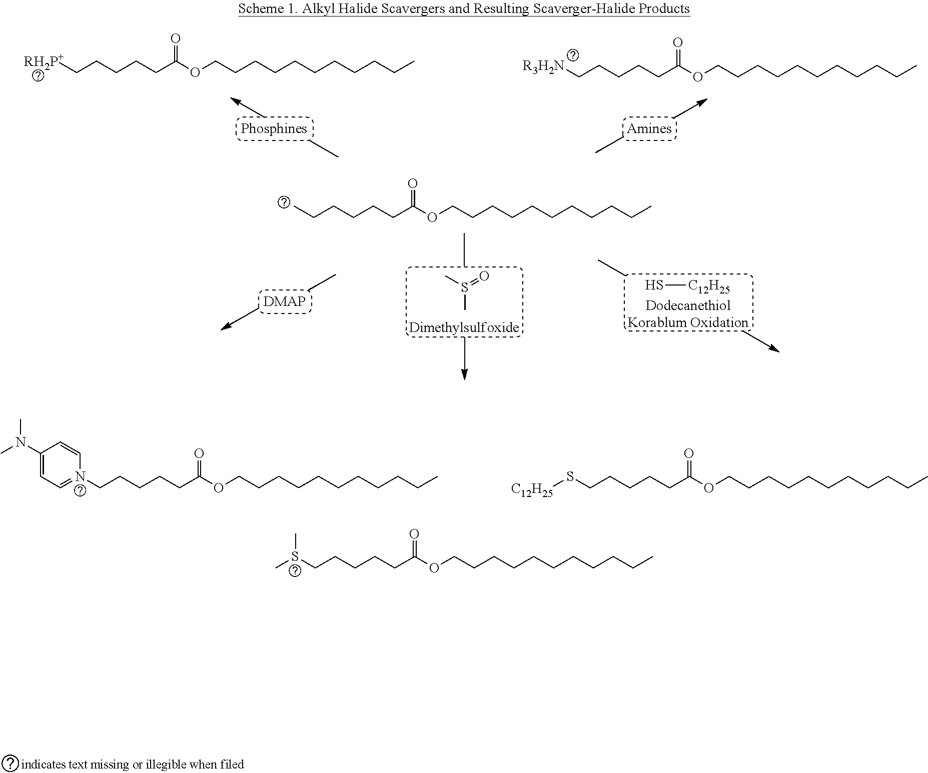

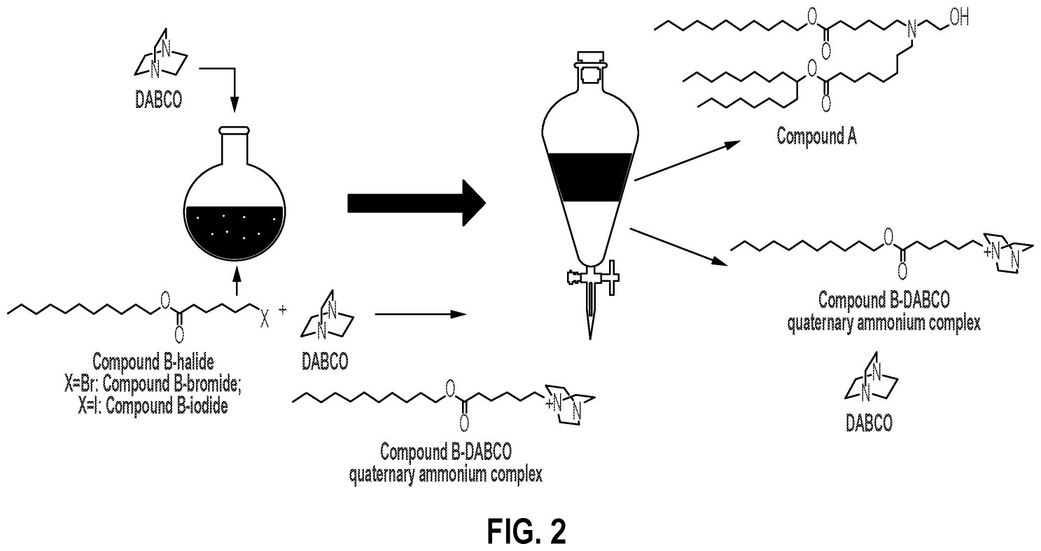

FIG. 2 schematically illustrates a halide scavenging-removal process according to the present disclosure, where the alkyl halide scavenger is DABCO and the alkyl halide is Compound B-halide. As shown in Scheme 2, DABCO is a highly nucleophilic tertiary amine which substitutes for the terminal halide to form a quaternary ammonium complex. Although IALs (e.g., Compound A) and alkyl halides (e.g., Compound B-halide) are soluble in nonpolar solvents, the quaternary ammonium complex is soluble in polar solvents (e.g., methanol, acetonitrile, water, or combinations thereof). Thus, the quaternary ammonium complex may be removed from a first composition comprising the IAL by liquid-liquid extraction with a polar solvent (e.g., acetonitrile) to produce a second composition comprising the IAL (and other impurities that may be present, such as aldehydes) in the nonpolar solvent (e.g., heptane). By removing the quaternary ammonium complex, the alkyl halide impurities are removed from the first composition comprising the IAL, such that the alkyl halide concentration in the second composition is less than in the first composition. Similar removal methods (extraction into polar solvents) would apply to the other halide-scavenger reaction products shown in Scheme 1.

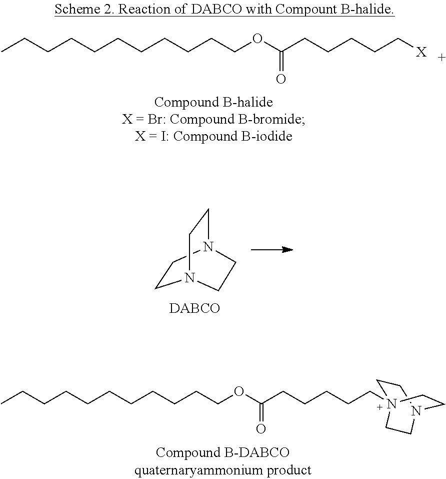

Typically, the halide scavenging step is conducted with an amount of scavenger effective to react with alkyl halide present in the composition, optionally with an amount of halide scavenger in excess of an amount required to react with all alkyl halide present in the composition (e.g., a stoichiometric excess of halide scavenger relative to alkyl halide).

The halide scavenging step is conducted at a temperature at which the intended reaction will occur, typically at a temperature at which the intended reaction will reach completion within a desired timeframe. In some embodiments, the halide scavenging step is conducted at room temperature. In other embodiments, the halide scavenging step is conducted at an elevated temperature. In other embodiments, the halide scavenging step is conducted at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 110° C., about 120° C., or any range or value therein between. In some embodiments, the halide scavenging step is conducted at a temperature of about 20° C. to about 100° C., about 30° C. to about 90° C., about 40° C. to about 80° C., about 50° C. to about 80° C., about 60° C. to about 80° C., about 65° C. to about 80° C., about 70° C. to about 80° C., or about 75° C. to about 80° C. In some embodiments, the halide scavenging step is conducted at a temperature of about 75° C. to about 80° C. As used herein, “conducted at a temperature of” means that the composition resulting from addition of the halide scavenger to the IAL composition is maintained at that temperature for a period of time.

Typically, the halide scavenging step is conducted for a time period sufficient for the intended reaction to reach completion. In some embodiments, the halide scavenging step is conducted for a duration of about 10 minutes, about 15 minutes, about 30 minutes, about 45 minutes, about 1 hour, about 1.5 hours, about 2 hours, about 2.5 hours, about 3 hours, about 3.5 hours, about 4 hours, about 4.5 hours, about 5 hours, about 6 hours, about 7 hours, about 8 hours, about 9 hours, about 10 hours, about 12 hours, about 16 hours, about 20 hours, about 24 hours, or any range or value therein between. In some embodiments, the alkyl halide scavenger is mixed with the first composition for a duration of at least about 1 hour, at least about 2 hours, at least about 3 hours, at least about 4 hours, at least about 5 hours, or any range or value therein between. In any embodiments, the halide scavenging step may comprise mixing or stirring the composition resulting from addition of the halide scavenger to the IAL composition, such as mixing or stirring the composition for a duration of time as described herein.

In some embodiments, the halide scavenger is DABCO, and is added to the first composition comprising the IAL and the alkyl halide at an amount of about 0.2 equivalents, and the halide scavenging step is conducted at a temperature of about 75° C. to about 80° C. for a duration of at least about 4 hours.

Following the halide scavenging reaction, the scavenger-halide product (e.g., a quaternary ammonium complex) is removed from the first composition, such as by liquid-liquid extraction. For instance, the first composition comprising the IAL and the scavenger-halide product in a nonpolar solvent (e.g., heptane) may be washed with a polar solvent (e.g., methanol, acetonitrile, or combinations thereof) to selectively remove the scavenger-halide product into the polar solvent.

In some embodiments, the halide scavenging-removal step reduces the alkyl halide concentration in the IAL composition such that the composition comprising the IAL has an alkyl halide concentration of less than or equal to 2,000 ppm, less than or equal to 1,500 ppm, less than or equal to 1,000 ppm, less than or equal to 900 ppm, less than or equal to 800 ppm, less than or equal to 700 ppm, less than or equal to 600 ppm, less than or equal to 500 ppm, less than or equal to 400 ppm, less than or equal to 300 ppm, less than or equal to 200 ppm, less than or equal to 100 ppm, less than or equal to 90 ppm, less than or equal to 80 ppm, less than or equal to 70 ppm, less than or equal to 60 ppm, less than or equal to 50 ppm, less than or equal to 40 ppm, less than or equal to 30 ppm, less than or equal to 20 ppm, less than or equal to 10 ppm, less than or equal to 5 ppm, less than or equal to 1 ppm, or any range or value therein between. In specific embodiments, the halide scavenging-removal step reduces the alkyl halide concentration in the IAL composition such that the composition comprising the IAL has an alkyl halide concentration of 100 ppm or less. In further specific embodiments, the halide scavenging-removal step reduces the alkyl halide concentration in the IAL composition such that the composition comprising the IAL has an alkyl halide concentration below the limit of quantitation, e.g., by UPLC-UV. In further specific embodiments, the halide scavenging-removal step reduces the alkyl halide concentration in the IAL composition such that the composition comprising the IAL has an alkyl halide concentration below the limit of detection, e.g., by UPLC-UV. The alkyl halide concentration of a composition may be determined by UPLC-UV.

In some embodiments, the halide scavenging-removal process further comprises, after the contacting sub-step and before the separating sub-step, filtering the composition to remove insoluble materials. In some embodiments, the filtering removes inorganic salts (e.g., carbonate salts such as K₂CO₃, halide salts such as KBr, KI, etc.). The filtering may be performed by any suitable method, including gravity filtration or vacuum filtration, optionally using a filtration medium or filtering aid, such as porous filtration media (e.g., CELITE®). In some embodiments, the filtering may be performed after the halide scavenging-removal step (e.g., by filtering the composition comprising the IAL after the scavenger-halide product has been separated therefrom).

### 4. Acid Extraction

In some embodiments, an acid extraction step is conducted, such as to alter the solubility of the IAL to facilitate a scavenging-extraction step that removes reactive impurities (e.g., aldehydes). For instance, the IAL and the reactive impurities (e.g., aldehydes) may be soluble in a first solvent (e.g., heptane). However, contacting the IAL, which is a tertiary amine, with an acid (e.g., a dicarboxylic acid) may produce an IAL salt, which has a solubility that is different from that of the IAL. Thus, an acid extraction step may effectively alter the solubility of the IAL, such that it becomes more soluble in a polar solvent (e.g., water, acetonitrile, methanol, or combinations thereof) than in a nonpolar solvent. Thus, contacting an IAL in a first (e.g., nonpolar) solvent with an acid and a second (e.g., polar) solvent extracts the IAL, in salt form, into the second (polar) solvent to produce an IAL salt composition (e.g., an IAL salt solution), while residual nonpolar reactive impurities (e.g., aldehydes) remain in the first (e.g., nonpolar) solvent (e.g., heptane). The first solvent containing the reactive impurities may then be removed and discarded (e.g., by liquid-liquid extraction, including washing the IAL salt solution with heptane). The IAL salt solution may then be neutralized, converting the IAL salt into the IAL, which is re-dissolved in a less polar solvent (e.g., heptane) or may be subjected to further scavenging-removal steps.

In some embodiments, an acid extraction step is conducted to remove other impurities, such as to remove nonpolar impurities. In such embodiments, after acid extraction that produces an IAL salt composition, nonpolar impurities can be separated from the IAL salt composition by methodologies parallel to those outlined above for nonpolar reactive impurities (e.g., aldehydes).

In any embodiments comprising an acid extraction, the acid may be any suitable acid for converting the IAL into an IAL salt. In some embodiments, the acid comprises or consists of one or more acids selected from inorganic acids (e.g., hydrochloric acid, hydrobromic acid, hydroiodic acid, sulfuric acid, nitric acid, boric acid, carbonic acid, hypophosphorous acid, phosphorous acid, and phosphoric acid) and organic acids (e.g., carboxylic acids such as acetic acid, aconitic acid (e.g., cis-aconitic acid or trans-aconitic acid), adipic acid, ascorbic acid (e.g., L-ascorbic acid), benzoic acid, citric acid, formic acid, glyceric acid, lactic acid, maleic acid, malic acid, malonic acid, methylmalonic acid, ethylmalonic acid, nicotinic acid, oxalic acid, propionic acid, phthalic acid, salicylic acid, sorbic acid, succinic acid, tartaric acid (e.g., (+)-L-tartaric acid), trimesic acid, butyric acid, valeric acid, hexanoic acid, heptanoic acid, octanoic acid, nonanoic acid, decanoic acid, undecanoic acid, dodecanoic acid); and sulfonic acids (e.g., methane sulfonic acid, ethane sulfonic acid, isethionic acid, toluenesulfonic acid, camphorsulfonic acid, etc.). In some embodiments, the acid comprises or consists of one or more acids selected from ethylmalonic acid, malonic acid, methanesulfonic acid, methylmalonic acid, and phosphoric acid. In some embodiments, the acid comprises or consists of malonic acid.

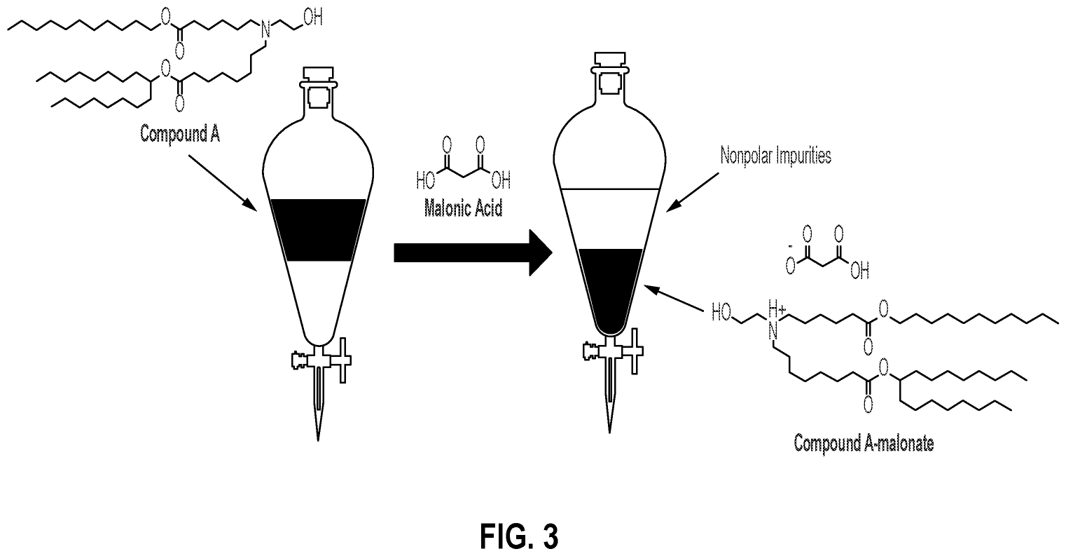

Scheme 3 shows an exemplary scheme for formation of an IAL salt when the IAL is Compound A and the acid is malonic acid. FIG. 3 schematically illustrates differences in solubility between the free IAL (soluble in nonpolar solvents) and the IAL-malonate salt (soluble in polar solvents).

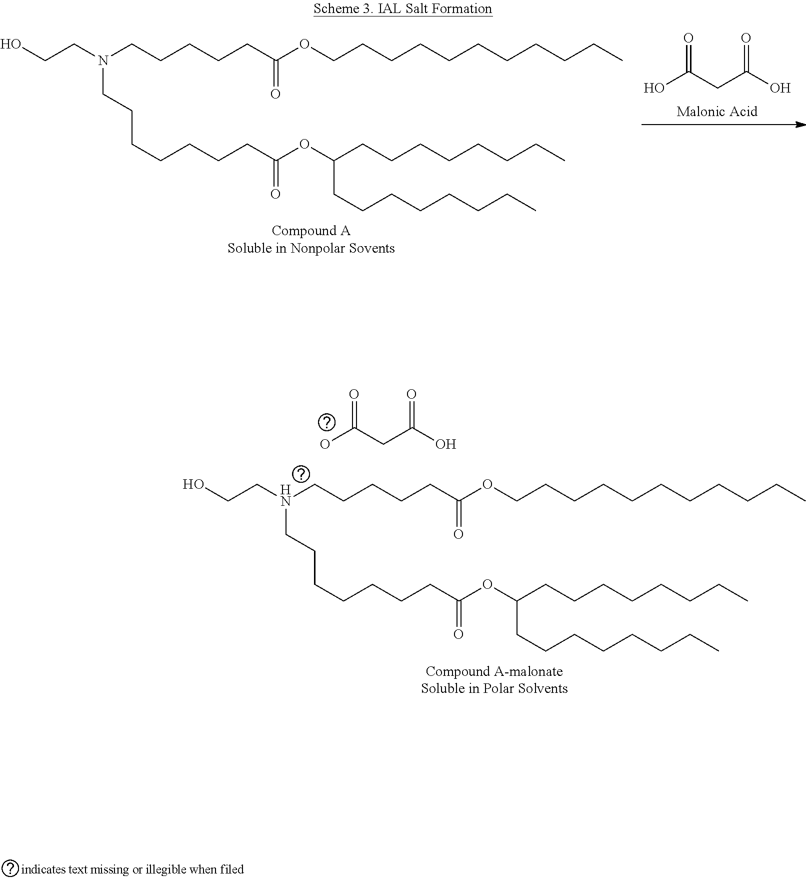

Typically, the acid is used in an amount effective to react with IAL present in the composition, optionally with an amount of acid in excess of an amount required to react with all IAL present in the composition, to convert the IAL into an IAL salt. In some embodiments, the acid is used in an amount of about 1.0 equivalents, about 1.1 equivalents, about 1.2 equivalents, about 1.25 equivalents, about 1.3 equivalents, about 1.4 equivalents, about 1.5 equivalents, about 1.6 equivalents, about 1.7 equivalents, about 1.8 equivalents, about 1.9 equivalents, about 2.0 equivalents, about 2.5 equivalents, about 3.0 equivalents, about 3.5 equivalents, about 4.0 equivalents, about 4.5 equivalents, about 5.0 equivalents, or about 10 equivalents, wherein “equivalents” refers to molar equivalents relative to the molar amount of IAL present, or any range or value therein between. In some embodiments, the acid is used in an amount of about 1.25 equivalents, relative to the molar amount of IAL present.

The solvent in which the IAL is soluble (sometimes referred to herein as the “first” solvent) may be any suitable solvent for preparing a solution of the IAL. In some embodiments, the first solvent is a nonpolar solvent (e.g., pentane, hexane, heptane, octane, or any combination thereof). In some embodiments, the first solvent is heptane. In some embodiments, reactive impurities (e.g., halides or carbonyls, such as aldehydes or ketones) also are soluble in the first solvent.

The solvent in which the IAL salt is soluble (sometimes referred to herein as the “second” solvent) may be any suitable solvent for preparing a solution of the IAL salt. In some embodiments, the second solvent is a polar solvent (e.g., water, ethanol, methanol, isopropanol, acetone, acetonitrile, dimethylformamide (DMF), dimethylsulfoxide (DMSO), or any combination thereof). In some embodiments, the second solvent is acetonitrile, methanol, or a combination thereof (e.g., 4:1 acetonitrile:methanol). In some embodiments, the reactive impurities are less soluble in the second solvent than in the first solvent, such that the IAL salt can be selectively extracted into the second solvent, with reactive impurities remaining in the first solvent.

In some embodiments, after the IAL salt is extracted into the second solvent and separated from the first solvent (containing reactive impurities) (which may be discarded), the resulting IAL salt composition (e.g., IAL salt solution) is washed with a solvent that selectively dissolves remaining impurities, such as remaining reactive impurities and other nonpolar impurities, but not the IAL salt. In some embodiments, such a wash solvent is the same as the first solvent (e.g., heptane). In some embodiments, such a wash solvent is any suitable nonpolar solvent (e.g., pentane, hexane, heptane, octane, or any combination thereof). In some embodiments, the reactive impurities are more soluble in the wash solvent than in the second solvent, such that the IAL salt remains in the second solvent, while the reactive impurities are extracted into the wash solvent.

In some embodiments, after the IAL salt is extracted into the second solvent and separated from the first solvent, and, optionally after a wash step as described above, the IAL salt is subject to a neutralization reaction to obtain the IAL. The neutralization reaction may be performed using any suitable neutralizing agent for converting the IAL salt to the IAL. In some embodiments, the neutralizing agent is one or more selected from hydroxides of alkali metals (e.g., NaOH, KOH) and salts of alkali metals (e.g., carbonates, hydrogen carbonates, sulfates, acetates, phosphates, etc.). In some embodiments, the neutralizing agent comprises or consists of K₃PO₄, KHCO₃, or a combination thereof. In some embodiments, the neutralizing agent comprises or consists of K₃PO₄. In some embodiments, the neutralizing agent comprises or consists of KHCO₃. In some embodiments, the neutralization reaction is performed after a second impurity scavenging step (e.g., an aldehyde scavenging step), as described in more detail below.

### 5. Carbonyl Scavenging-Removal

In some embodiments, carbonyl impurities (e.g., aldehydes, ketones, or combinations thereof) are removed from a composition comprising the IAL or IAL salt (e.g., an IAL salt composition as described above) by contacting the composition comprising the IAL or IAL salt with a carbonyl scavenger (e.g., an aldehyde scavenger).

For purposes of this disclosure, a “carbonyl scavenger” is a compound that selectively reacts with a carbonyl impurity (over the IAL or IAL salt) to produce a scavenger-carbonyl product that has a different solubility from the IAL or IAL salt. For purposes of this disclosure, an “aldehyde scavenger” is a compound that selectively reacts with an aldehyde impurity (over the IAL or IAL salt) to produce a scavenger-aldehyde product that has a different solubility from the IAL or IAL salt. Similarly, for purposes of this disclosure, a “ketone scavenger” is a compound that selectively reacts with a ketone impurity (over the IAL or IAL salt) to produce a scavenger-ketone product that has a different solubility from the IAL or IAL salt. In some embodiments, the carbonyl scavenger comprises one or more aldehyde scavengers. In some embodiments, the carbonyl scavenger comprises one or more ketone scavengers. In some embodiments, the same compound may be an aldehyde scavenger and a ketone scavenger.

For instance, a carbonyl scavenger may react with a carbonyl (e.g., aldehyde or ketone) soluble in a polar solvent (e.g., acetonitrile, methanol, water, or combinations thereof) to produce a scavenger-carbonyl product (e.g., an oxime, Schiff base, or a scavenger-carbonyl adduct) that is more soluble in a polar solvent (e.g., acetonitrile, methanol, water, or combinations thereof) than in a nonpolar solvent (e.g., heptane). In some embodiments, the scavenger-carbonyl product is a scavenger-carbonyl adduct (e.g., a scavenger-aldehyde adduct or scavenger-ketone adduct). In some embodiments, the scavenger-carbonyl product is more soluble in polar solvents than in nonpolar solvents, while the IAL is more soluble in nonpolar solvents than in polar solvents. The term “scavenger-carbonyl adduct” refers to an addition product of the carbonyl scavenger and the carbonyl impurity that retains all atoms of the scavenger and carbonyl impurity. Likewise, the term “scavenger-aldehyde adduct” refers to an addition product of an aldehyde scavenger and an aldehyde impurity that retains all atoms of the scavenger and aldehyde impurity. Similarly, the term “scavenger-ketone adduct” refers to an addition product of a ketone scavenger and a ketone impurity that retains all atoms of the scavenger and ketone impurity.

In some embodiments, the scavenger-carbonyl product is more soluble in polar solvents than in nonpolar solvents. In some embodiments, under conditions that convert the IAL salt (soluble in polar solvents) to the free IAL (soluble in nonpolar solvents) by a neutralizing agent, the scavenger-carbonyl product is converted to a derivative of the scavenger-carbonyl product that is more soluble in polar solvents than in nonpolar solvents (e.g., a salt of a scavenger-aldehyde product). This facilitates separation of the scavenger-carbonyl product from the IAL. Thus, in some embodiments, free IAL may be separated from the scavenger-carbonyl product or derivative of the scavenger-carbonyl product using the selective solubility of the scavenger-carbonyl product or derivative of the scavenger-carbonyl product in polar solvents over nonpolar solvents, while free IAL is more soluble in nonpolar solvents than in polar solvents. In some embodiments, the scavenger-carbonyl product is an oxime compound and is converted to an oxime salt under conditions effective to convert the IAL salt to the free IAL.

Non-limiting examples of suitable carbonyl scavengers (e.g., aldehyde scavengers) are shown in Scheme 4, along with exemplary scavenger-aldehyde products.

In some embodiments, the carbonyl scavenger comprises or consists of one or more selected from O-hydroxylamines (e.g., 2-(aminooxy)acetic acid, O-benzylhydroxylamine (“O-BHA”), O-tritylhydroxylamine, 0-(2,3,4,5,6-pentafluorobenzyl)hydroxylamine (“PFBHA”), a p-benzyloxybenzyl alcohol-based hydroxylamine (Wang resin hydroxylamine), or salts thereof); N-hydroxylamines (e.g., N-benzylhydroxylamine or salts thereof), aminobenzamides, cysteine (e.g., L-cysteine), and cysteine analogs. In some embodiments, the carbonyl scavenger comprises or consists of one or more selected from O-benzylhydroxylamine (O-BHA), N-benzylhydroxylamine, 2-(aminooxy)acetic acid, O-tritylhydroxylamine, O-(2,3,4,5,6-pentafluorobenzyl)hydroxylamine (PFBHA), p-benzyloxybenzyl alcohol-based hydroxylamines and salts thereof, aminobenzamides, cysteines and cysteine analogs. In some embodiments, the carbonyl scavenger comprises or consists of one or more selected from O-benzylhydroxylamine (O-BHA), N-benzylhydroxylamine, and 2-(aminooxy)acetic acid. In some embodiments, the carbonyl scavenger comprises or consists of 2-(aminooxy)acetic acid.

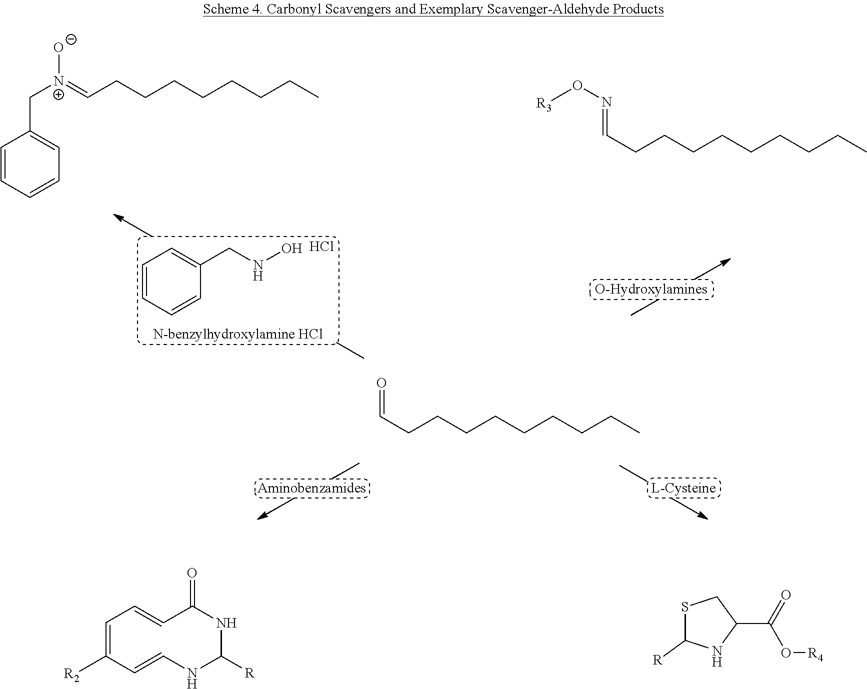

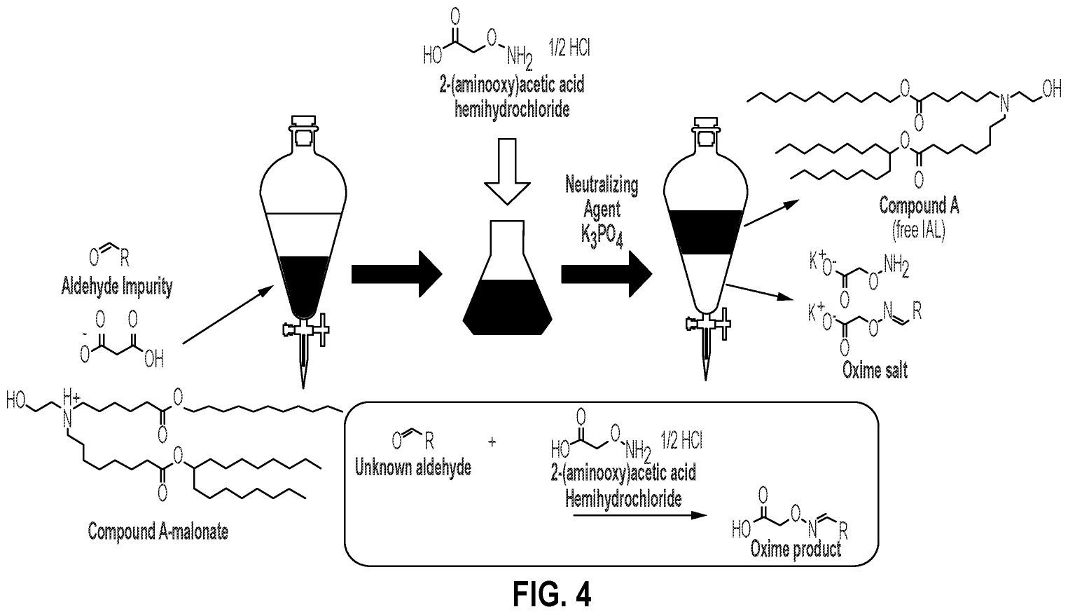

FIG. 4 schematically illustrates an aldehyde scavenging-removal process according to the present disclosure, where the aldehyde scavenger is 2-(aminooxy)acetic acid. The formation of one potential genre of scavenger-aldehyde products (oximes) is shown in Scheme 5. The IAL salt is soluble in polar solvents (e.g., acetonitrile, methanol, or combinations thereof). When the IAL salt is neutralized and converted to the free IAL form (which is soluble in nonpolar solvents), the oxime reaction products may be converted to oxime salts (which are soluble in polar solvents). Thus, oxime products (present as salts) can be separated from the IAL based on their different solubility. For example, oxime products (present as salts) can be extracted into or washed with a polar solvent, while the IAL remains present in a nonpolar solvent (e.g., heptane). The polar solvent, containing the oxime product (and possibly excess scavenger), may be discarded. Similar neutralization-extraction steps may be applied to the other scavenger-carbonyl products shown in Scheme 4.

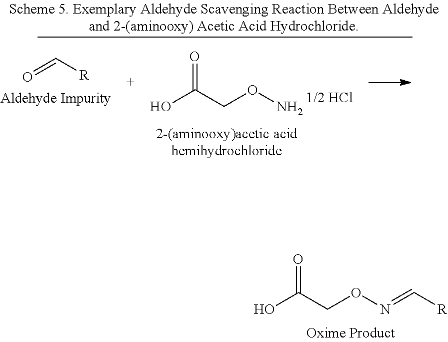

Typically, the carbonyl scavenging step is conducted with an amount of scavenger effective to react with carbonyls (aldehydes, ketones, or combinations thereof) present in the composition, optionally with an amount of carbonyl scavenger in excess of an amount required to react with all carbonyl impurities present in the composition (e.g., a stoichiometric excess of carbonyl scavenger relative to carbonyls). For instance, an aldehyde scavenging step may be conducted with an amount of aldehyde scavenger effective to react with all aldehyde present in the composition, optionally with an amount of aldehyde scavenger in excess of an amount required to react with all aldehyde impurities present in the composition. Similarly, a ketone scavenging step may be conducted with an amount of ketone scavenger effective to react with all ketone present in the composition, optionally with an amount of ketone scavenger in excess of an amount required to react with all ketone impurities present in the composition.

The carbonyl scavenging step is conducted at a temperature at which the intended reaction will occur, typically at a temperature at which the intended reaction will reach completion within a desired timeframe. In some embodiments, the carbonyl scavenging step is conducted at room temperature. In other embodiments, the carbonyl scavenging step is conducted at an elevated temperature. In other embodiments, the carbonyl scavenging step is conducted at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 110° C., about 120° C., or any range or value therein between. In some embodiments, the carbonyl scavenger is mixed with the IAL salt solution at a temperature of about 20° C. to about 100° C., about 30° C. to about 90° C., about 40° C. to about 80° C., about 50° C. to about 80° C., about 60° C. to about 80° C., about 65° C. to about 80° C., about 70° C. to about 80° C., or about 75° C. to about 80° C. In some embodiments, the carbonyl scavenging step (e.g., aldehyde scavenging step and/or ketone scavenging step) is conducted by mixing the carbonyl scavenger (e.g., aldehyde scavenger and/or ketone scavenger) with the IAL salt solution at room temperature. As used herein, “conducted at a temperature of” means that the composition resulting from addition of the carbonyl scavenger to the IAL composition is maintained at that temperature for a period of time.

Typically, the carbonyl scavenging step (e.g., aldehyde scavenging step and/or ketone scavenging step) is conducted for a time period sufficient for the intended reaction to reach completion. In some embodiments, the carbonyl scavenging step is conducted for a duration of about 10 minutes, about 15 minutes, about 30 minutes, about 45 minutes, about 1 hour, about 1.5 hours, about 2 hours, about 2.5 hours, about 3 hours, about 3.5 hours, about 4 hours, about 4.5 hours, about 5 hours, about 6 hours, about 7 hours, about 8 hours, about 9 hours, about 10 hours, about 12 hours, about 16 hours, about 20 hours, about 24 hours, or any range or value therein between. In some embodiments, the carbonyl scavenging step is conducted for a duration of at least about 1 hour, at least about 2 hours, at least about 3 hours, at least about 4 hours, at least about 5 hours, or any range or value therein between. In some embodiments, the carbonyl scavenging step is conducted for a duration of at least about 2 hours. In any embodiments, the carbonyl scavenging step may comprise mixing or stirring the composition resulting from addition of the carbonyl scavenger to the IAL composition, such as mixing or stirring the composition for a duration of time as described herein.

In some embodiments, the carbonyl scavenging-removal step reduces the carbonyl concentration in the IAL composition such that the molar ratio of carbonyl impurity to the IAL is less than or equal to 5×10⁻³, less than or equal to 4×10⁻³, less than or equal to 3×10⁻³, less than or equal to 2×10⁻³, less than or equal to 1×10⁻³, less than or equal to 9×10⁻⁴, less than or equal to 8×10⁻⁴, less than or equal to 7×10⁻⁴, less than or equal to 6×10⁻⁴, less than or equal to 5×10⁻⁴, less than or equal to 4×10⁻⁴, less than or equal to 3×10⁻⁴, less than or equal to 2×10⁻⁴, less than or equal to 1×10⁻⁴, or any range or value therein between. In some embodiments, the carbonyl concentration (e.g., aldehyde concentration or ketone concentration) may be determined using a LC-UV assay.

In some embodiments, wherein the carbonyl scavenging-removal step is an aldehyde scavenging removal step, the aldehyde scavenging-removal step reduces the aldehyde concentration in the IAL composition such that the molar ratio of aldehyde impurity to the IAL is less than or equal to 5×10⁻³, less than or equal to 4×10⁻³, less than or equal to 3×10⁻³, less than or equal to 2×10⁻³, less than or equal to 1×10⁻³, less than or equal to 9×10⁻⁴, less than or equal to 8×10⁻⁴, less than or equal to 7×10⁻⁴, less than or equal to 6×10⁻⁴, less than or equal to 5×10⁻⁴, less than or equal to 4×10⁻⁴, less than or equal to 3×10⁻⁴, less than or equal to 2×10⁻⁴, less than or equal to 1×10⁻⁴, or any range or value therein between, as may be determined using a LC-UV assay. In specific embodiments, wherein the carbonyl scavenging-removal step is an aldehyde scavenging removal step, the aldehyde scavenging-removal step reduces the aldehyde concentration in the IAL composition such that the molar ratio of reactive aldehyde impurity to the IAL is 2×10⁻³ or less. In specific embodiments, wherein the carbonyl scavenging-removal step is an aldehyde scavenging removal step, the aldehyde scavenging-removal step reduces the aldehyde concentration in the IAL composition to below the limit of quantification. In specific embodiments, wherein the carbonyl scavenging-removal step is an aldehyde scavenging removal step, the aldehyde scavenging-removal step reduces the aldehyde concentration in the IAL composition to below the limit of detection.

In some embodiments, wherein the carbonyl scavenging-removal step is a ketone scavenging removal step, the ketone scavenging-removal step reduces the ketone concentration in the IAL composition such that the molar ratio of ketone impurity to the IAL is less than or equal to 5×10⁻³, less than or equal to 4×10⁻³, less than or equal to 3×10⁻³, less than or equal to 2×10⁻³, less than or equal to 1×10⁻³, less than or equal to 9×10⁻⁴, less than or equal to 8×10⁻⁴, less than or equal to 7×10⁻⁴, less than or equal to 6×10⁻⁴, less than or equal to 5×10⁻⁴, less than or equal to 4×10⁻⁴, less than or equal to 3×10⁻⁴, less than or equal to 2×10⁻⁴, less than or equal to 1×10⁻⁴, or any range or value therein between, as may be determined using a LC-UV assay. In specific embodiments, wherein the carbonyl scavenging-removal step is a ketone scavenging removal step, the ketone scavenging-removal step reduces the ketone concentration in the IAL composition to below the limit of quantification. In specific embodiments, wherein the carbonyl scavenging-removal step is a ketone scavenging removal step, the ketone scavenging-removal step reduces the ketone concentration in the IAL composition to below the limit of detection.

In some embodiments, the carbonyl scavenger comprises or consists of an aldehyde scavenger. In some embodiments, the aldehyde scavenger comprises or consists of 2-(aminooxy)acetic acid, used in a stoichiometric excess relative to the amount of aldehyde in the IAL salt solution, and the aldehyde scavenging step is conducted by mixing the 2-(aminooxy)acetic acid with the IAL salt solution at room temperature for at least about 2 hours.

### 6. Purified IAL Composition

The methods disclosed herein produce a purified IAL composition comprising purified IAL that has a reduced concentration of reactive impurities compared to a crude IAL composition (e.g., the first composition comprising the IAL that is subjected to the methods disclosed herein). In some embodiments, the purity of the IAL in the purified IAL composition is increased relative to the first composition. In some embodiments, the purity of the IAL is at least about 95%, at least about 95.5%, at least about 96%, at least about 96.5%, at least about 97%, at least about 97.5%, at least about 98%, at least about 98.5%, at least about 99%, at least about 99.5%, at least about 99.6%, at least about 99.7%, at least about 99.8%, or at least about 99.9%. In some embodiments, the IAL purity is determined by ultra-performance liquid chromatography with charged aerosol detection (UPLC-CAD).

The methods disclosed herein produce a purified IAL composition that is less susceptible to lipid-polynucleotide adduct (e.g., lipid-mRNA adduct) formation. In some embodiments, the adduct formation rate of a composition comprising a purified IAL composition as described herein and a polynucleotide, such as mRNA, relative to the total amount of polynucleotide (e.g., mRNA), is less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, or any range or value therein between. In some embodiments, the amount of lipid-polynucleotide adduct impurity (e.g., aldehyde-polynucleotide adduct impurity or halide-polynucleotide adduct impurity, etc.) present in a composition may be measured by reverse phase ion pair chromatography (RP-IP HPLC), as described in U.S. application Ser. No. 17/508,786 and Packer et al., Nature Comm. (2021) 12: 6777. A lipid binary assay discussed therein was developed in order to determine levels of reactive impurities.

### 7. Delivery Agents Comprising Purified Ionizable Lipids

In some embodiments, the compositions or formulations of the present disclosure comprise a delivery agent, e.g., a liposome, a lipolex, a lipid nanoparticle (LNP), or any combination thereof. The polynucleotides described herein can be formulated using one or more liposomes, lipoplexes, or lipid nanoparticles. Liposomes, lipoplexes, or lipid nanoparticles can be used to improve the efficacy of the polynucleotides directed protein production as these formulations can increase cell transfection by the polynucleotide; and/or increase the translation of encoded protein. The liposomes, lipoplexes, or lipid nanoparticles can also be used to increase the stability of the polynucleotides.

IALs purified by the methods described herein can be used to prepare delivery agents, including LNPs, liposomes, lipoplexes, and compositions comprising the same, which can in turn be used to prepare lipid delivery agent-polynucleotide products (e.g., LNP-polynucleotide products), including pharmaceutical compositions comprising LNPs comprising mRNA and purified IALs.

### (a) Lipid Nanoparticle Compositions

Some embodiments relate to a lipid nanoparticle composition comprising a polynucleotide and an ionizable lipid (e.g., IAL) obtained using the purification methods disclosed herein. In some embodiments, the LNP composition comprises less than about 10% of an ionizable lipid-polynucleotide adduct impurity relative to a total amount of the polynucleotide. In some embodiments, the composition comprises less than about 5%, less than about 3%, less than about 2%, or less than about 1% of ionizable lipid-polynucleotide adduct impurity relative to the total amount of polynucleotides. In some embodiments, the composition is substantially free of ionizable lipid-polynucleotide adduct impurity.

The term “adduct” or “adduct impurity” refers to the covalent addition of a lipid or other entity (e.g., hydrophobic entity) or polymer chain to a polynucleotide, such as mRNA. An “ionizable lipid-polynucleotide adduct impurity” (also referred to herein as “impurity group” or “IG”) is a type of adduct that comprises the covalent modification of a polynucleotide (e.g., in a LNP) with (i) an ionizable lipid; (ii) a derivative thereof (such as a secondary amine or reactive aldehyde produced from decomposed ionizable lipids), or (iii) residual reactive impurities, starting materials, or side products (which may include alkyl halides and carbonyl-containing compounds such as aldehydes and ketones) which may originate from the synthesis of ionizable lipids (e.g., IALs) and which may remain in a composition with the ionizable lipid (e.g., IAL) after the synthesis.

The presence of adduct impurities can lead to low translation competency of the polynucleotide of the adduct impurity. For instance, in the context of mRNA an adduct includes covalent modification of the mRNA in such away as to prevent translation of the mRNA. Without being bound by any particular theory, the low translation competency of adduct impurity polynucleotides may be due to chemical modifications across the length of the polynucleotide in the form of the polynucleotide-aldehyde (unbranched and/or branched) adduct species. Translation competency may be determined by assays known in the art and described herein (see, e.g., the working examples below). For example, translation competency may be determined by biochemical characterization with cell-free translation systems, fluorescently activated cell sorting (FACs) assays, and/or ribosomal-profiling of reporter mRNAs comprising adduct impurities.

In some embodiments, adduct impurities are formed as a result of the reaction between (i) polynucleotides and (ii) secondary amine and/or reactive aldehyde species produced from decomposed ionizable lipids. In some embodiments, adduct impurities are formed as a result of the reaction between (i) polynucleotides and (iii) residual reactive impurities, starting materials, or side products (which may include alkyl halides and carbonyl-containing compounds such as aldehydes and ketones) which originate from the synthesis of ionizable lipids (e.g., IALs) and which may remain in a composition with the ionizable lipid (e.g., IAL) after the synthesis.

In some embodiments, the amount of ionizable lipid-polynucleotide impurity in a composition may be measured by (RP-IP HPLC), such as described in U.S. application Ser. No. 17/508,786 and Packer et al., Nature Comm. (2021) 12: 6777.

The adduct impurity may comprise carbon chains covalently appended to the polynucleotides. The carbon chains, which are believed to be derived from the ionizable lipids, may be saturated or unsaturated and of various lengths. In some embodiments, the carbon chain is a C₆₋₃₀ carbon chain. In some embodiments, one or more covalently-appended lipids or derivatives thereof in the adduct impurity comprises a C₆₋₃₀ saturated carbon chain. In some embodiments, one or more covalently-appended lipids or derivatives thereof in the adduct impurity comprises a C₆₋₃₀ unsaturated carbon chain.

In some embodiments, one or more covalently-appended lipids or derivatives thereof in the adduct impurity comprises a carbon chain interrupted by a non-carbon group. In some embodiments, the carbon chain of an adduct impurity is interrupted by a —C(O)O— ester group. In some embodiments, one or more covalently-appended lipids or derivatives thereof in the adduct impurity comprises a C₆₋₃₀ saturated carbon chain interrupted by a —C(O)O— ester group. In some embodiments, one or more covalently-appended lipids or derivatives thereof in the adduct impurity comprises a C₆₋₃₀ unsaturated carbon chain interrupted by a —C(O)O— ester group.

In some embodiments, one or more covalently-appended lipids or derivatives thereof in the adduct impurity is attached to a nucleobase. Exemplary nucleobases include, but are not limited to, guanosine, cytidine, and methyl pseudouridine.

In some embodiments, a lipid nanoparticle composition comprises less than about 10%, less than about 5%, less than about 3%, less than about 2%, or less than about 1% of ionizable lipid-polynucleotide adduct impurity relative to the total amount of polynucleotides. In some embodiments, the composition comprises less than 10%, less than 5%, less than 3%, less than 2%, or less than 1% of ionizable lipid-polynucleotide adduct impurity relative to the total amount of polynucleotides. In some embodiments, the composition comprises less than about 10% of ionizable lipid-polynucleotide adduct impurity relative to the amount of polynucleotides. In some embodiments, the composition comprises less than about 5% of ionizable lipid-polynucleotide adduct impurity relative to the total amount of polynucleotides. In some embodiments, the composition comprises less than about 3% of ionizable lipid-polynucleotide adduct impurity relative to the total amount of polynucleotides. In some embodiments, the composition comprises less than about 2% of ionizable lipid-polynucleotide adduct impurity relative to the total amount of polynucleotides. In some embodiments, the composition comprises less than about 1% of ionizable lipid-polynucleotide adduct impurity relative to the total amount of polynucleotides.

In some embodiments, the composition is substantially free of ionizable lipid-polynucleotide adduct impurity. In some embodiments, a composition is “substantially free” of ionizable lipid-polynucleotide adduct impurity when the percent of adduct impurity in the composition relative to the total amount of polynucleotides is less than 0.5%, less than 0.4%, less than 0.3%, less than 0.2%, less than 0.1%, less than 0.05%, less than 0.01%, or less than 0.001%. In some embodiments, the composition is free of ionizable lipid-polynucleotide adduct impurity.

In some embodiments, the composition comprises between 0% and 10%, between 1% and 9%, between 2% and 8%, between 3% and 7%, or between 4% and 6% of ionizable lipid-polynucleotide adduct impurity relative to the total amount of polynucleotides. In some embodiments, the composition comprises between 1% and 10%, between 1% and 7%, between 1% and 5%, or between 1% and 3% or between 4% and 6% of ionizable lipid-polynucleotide adduct impurity relative to the total amount of polynucleotides.

The LNP compositions can also be advantageous in that the amount of adduct impurity in the composition does not increase substantially over time or under different storage conditions. For example, in some embodiments, the amount of adduct impurity in an LNP composition increases at an average rate of less than about 2%, less than about 1%, less than about 0.5%, or less than about 0.2% per day when stored at a temperature at about 40° C. or below (such as when stored at a temperature of about 25° C., about 20° C., about 15° C., about 10° C., about 8° C., about 5° C., about 2° C., about 0° C., about −10° C., or about −20° C.

In some embodiments, the amount of adduct impurity in an LNP composition increases at an average rate of less than 2%, less than 1%, less than 0.5%, or less than 0.2% per day when stored at a temperature at about 25° C. or below. In some embodiments, the amount of adduct does not substantially increase when stored at a temperature at about 25° C. or below (e.g., does not increase by more than 0.05%, more than 0.01%, more than 0.005%, or more than 0.001%).

The average rate of increase of adduct impurity group at various temperatures can be measured over a period of time, e.g., 2-10 consecutive dates, 2-5 consecutive days, 5-7 consecutive days, or 7-10 consecutive days, starting on the day (t=0), or one day after (t=1), the lipid nanoparticle composition was formed. In some embodiments, the average rate of increase of adduct impurity is measured over 2-5 consecutive days starting on the day the lipid nanoparticle composition was formed. In some embodiments, the average rate of increase of adduct impurity is measured over 2-5 consecutive days starting on one day after the lipid nanoparticle composition was formed. In some embodiments, the average rate of increase of adduct impurity is measured over 7-10 consecutive days starting on the day the lipid nanoparticle composition was formed. In some embodiments, the average rate of increase of adduct impurity is measured over 7-10 consecutive days starting on one day after the lipid nanoparticle composition was formed.

Some embodiments comprise adjusting the buffer or pH of the composition to reduce the amount of adduct impurity formed in the LNP composition (e.g., to inhibit ionizable lipid decomposition). For example, some embodiments comprise a composition with a TRIS (tris(hydroxymethyl)aminomethane) buffer at a concentration of about 10 mM or more, such as a concentration of about 20 mM, about 30 mM, about 50 mM, about 60 mM, about 75 mM, about 100 mM, about 120 mM, or about 150 mM TRIS buffer. In some embodiments, the composition comprises from about 10 mM to about 150 mM TRIS, such as from about 15 mM to about 120 mM TRIS or about 20 mM to about 100 mM TRIS. In some embodiments, the composition does not contain a PBS buffer.

In some embodiments, the composition is at a pH of from about 6.5 to about 9.0, such as about 7-8, about 7-7.5, about 7.4, or about 7.5.

The composition may also comprise a free reducing agent or antioxidant. Exemplary free reducing agents or antioxidants include, but are not limited to, potassium metabisulfite, sodium thioglycolate, tris(2-carboxyethyl)phosphine (TCEP), sodium thiosulfate, N-acetyl cysteine, glutathione, dithiothreitol (DTT), cystamine, dithioerythritol (DTE), dichlorodiphenyltrichloroethane (DDT), homocysteine, and lipoic acid.

Some embodiments comprise reducing the presence of trace metals in the composition (e.g., to inhibit ionizable lipid decomposition). Thus, the amount of transition metals in the LNP composition may be modified to reduce the amount of adduct impurity formed in the LNP composition. In some embodiments, the LNP composition comprises an amount of transition metals that is less than about 500 ppm, less than about 250 ppm, less than about 100 ppm, or less than about 50 ppm. In some embodiments, the LNP composition comprises an amount of transition metals that is less than 500 ppm, less than 250 ppm, less than 100 ppm, or less than 50 ppm. In some embodiments, the LNP composition comprises an amount of transition metals that is between 5 ppm and 500 ppm, between 25 ppm and 250 ppm, or between 50 and 100 ppm. In some embodiments, the LNP composition comprises an amount of transition metals that is between 0 ppm and 50 ppm, between 50 ppm and 100 ppm, between 100 ppm and 200 ppm, between 200 ppm and 300 ppm, between 300 ppm and 400 ppm, or between 400 ppm and 500 ppm. In some embodiments, the composition is substantially free of transition metals (e.g., the amount of transition metals is less than 5 ppm, less than 4 ppm, less than 3 ppm less than 2 ppm, less than 1 ppm, less than 0.1 ppm, less than 0.05 ppm, or less than 0.01 ppm). Exemplary transition metals include, but are not limited to, Pd, Cu, Fe, Ni, Pb, and Mn. In some embodiments, the composition comprises Fe. In some embodiments, the Fe has an oxidation state of 2+.

Some embodiments comprise reducing the presence of N-oxide compounds in the composition. In some embodiments, the N-oxide compound is an ionizable lipid in the LNP which has been oxidized to form an N-oxide group. Thus, the amount of N-oxide compound in the LNP composition may be modified to reduce the amount of adduct impurity formed in the LNP composition. In some embodiments, the LNP composition comprises an amount of N-oxide compound that is less than about 500 ppm, less than about 250 ppm, less than about 100 ppm, or less than about 50 ppm. In some embodiments, the LNP composition comprises an amount of N-oxide compound that is less than 500 ppm, less than 250 ppm, less than 100 ppm, or less than 50 ppm. In some embodiments, the LNP composition comprises an amount of N-oxide compound that is between 5 ppm and 500 ppm, between 25 ppm and 250 ppm, or between 50 and 100 ppm. In some embodiments, the LNP composition comprises an amount of N-oxide compound that is between 0 ppm and 50 ppm, between 50 ppm and 100 ppm, between 100 ppm and 200 ppm, between 200 ppm and 300 ppm, between 300 ppm and 400 ppm, or between 400 ppm and 500 ppm. In some embodiments, the composition is substantially free of N-oxide compounds (e.g., the amount of N-oxide compound is less than 5 ppm, less than 4 ppm, less than 3 ppm less than 2 ppm, less than 1 ppm, less than 0.1 ppm, less than 0.05 ppm, or less than 0.01 ppm).

In some embodiments, the LNP composition comprises an amount of lipid aldehyde that is less than about 500 ppm, less than about 250 ppm, less than about 100 ppm, or less than about 50 ppm. In some embodiments, the LNP composition comprises an amount of lipid aldehyde that is less than 500 ppm, less than 250 ppm, less than 100 ppm, or less than 50 ppm. In some embodiments, the LNP composition comprises an amount of lipid aldehyde that is between 5 ppm and 500 ppm, between 25 ppm and 250 ppm, or between 50 and 100 ppm. In some embodiments, the LNP composition comprises an amount of lipid aldehyde that is between 0 ppm and 50 ppm, between 50 ppm and 100 ppm, between 100 ppm and 200 ppm, between 200 ppm and 300 ppm, between 300 ppm and 400 ppm, or between 400 ppm and 500 ppm. In some embodiments, the composition is substantially free of lipid aldehyde (e.g., the amount of lipid aldehyde is less than 5 ppm, less than 4 ppm, less than 3 ppm less than 2 ppm, less than 1 ppm, less than 0.1 ppm, less than 0.05 ppm, or less than 0.01 ppm).

In some embodiments, the LNP composition is in liquid form.

a. Lipid Nanoparticles

Lipid nanoparticles (LNPs) typically comprise one or more lipids and a nucleic acid cargo (i.e., polynucleotide) of interest. In some embodiments, the lipid is an ionizable lipid (e.g., an ionizable amino lipid), sometimes referred to in the art as an “ionizable cationic lipid”. In some embodiments, lipid nanoparticles further comprise other components, including a phospholipid, a structural lipid, and a molecule capable of reducing particle aggregation, for example a PEG or PEG-modified lipid. In some embodiments, the lipid nanoparticle comprises at least one ionizable cationic lipid, at least one non-cationic lipid, at least one sterol, and/or at least one polyethylene glycol (PEG)-modified lipid. The lipid nanoparticles can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491.

In some embodiments, the lipid nanoparticle is a lipid nanoparticle described in Intl. Pub. Nos. WO2013123523, WO2012170930, WO2011127255, WO2008103276; or U.S. Pub. No. US20130171646.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, the lipid nanoparticles described herein have a diameter from about 1 nm to about 100 nm such as, but not limited to, about 1 nm to about 20 nm, from about 1 nm to about 30 nm, from about 1 nm to about 40 nm, from about 1 nm to about 50 nm, from about 1 nm to about 60 nm, from about I nm to about 70 nm, from about I nm to about 80 nm, from about I nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles described herein have a diameter from about 10 to 500 nm. In some embodiments, the lipid nanoparticle can have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

The ratio between the lipid composition and the polynucleotide can be from about 10:1 to about 60:1 (wt/wt). In some embodiments, the ratio between the lipid composition and the polynucleotide can be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the polynucleotide encoding a therapeutic agent is about 20:1 or about 15:1.

In some embodiments, the lipid nanoparticles described herein can comprise polynucleotides (e.g., mRNA) in a lipid:polynucleotide weight ratio of 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1, from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In some embodiments, the lipid nanoparticles described herein can comprise the polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

In some embodiments, the pharmaceutical composition disclosed herein can contain more than one polynucleotide. For example, a composition or pharmaceutical composition disclosed herein can contain two or more polynucleotides (e.g., RNA, e.g., mRNA) formulated in the same lipid nanoparticle.

The lipid nanoparticles described herein can be geometrically engineered to modulate macrophage and/or the immune response. The geometrically engineered particles can have varied shapes, sizes and/or surface charges to incorporate the polynucleotides described herein for targeted delivery such as, but not limited to, pulmonary delivery (see, e.g., Intl. Pub. No. WO 2013/082111). Other physical features the geometrically engineering particles can include are, but are not limited to, fenestrations, angled arms, asymmetry and surface roughness, and charge that can alter the interactions with cells and tissues.

In some embodiments, the lipid nanoparticles described herein are stealth nanoparticles or target-specific stealth nanoparticles such as, but not limited to, those described in U.S. Pub. No. US20130172406. The stealth or target-specific stealth nanoparticles can comprise a polymeric matrix, which can comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates, or combinations thereof.

The LNPs can be prepared using microfluidic mixers or micromixers. Exemplary microfluidic mixers can include, but are not limited to, a slit interdigital micromixer including, but not limited to those manufactured by Microinnova (Allerheiligen bei Wildon, Austria) and/or a staggered herringbone micromixer (SHM) (see Zhigaltsev et al., “Bottom-up design and synthesis of limit size lipid nanoparticle systems with aqueous and triglyceride cores using millisecond microfluidic mixing,” Langmuir 28:3633-40 (2012); Belliveau et al., “Microfluidic synthesis of highly potent limit-size lipid nanoparticles for in vivo delivery of siRNA,” Molecular Therapy-Nucleic Acids. 1:e37 (2012); Chen et al., “Rapid discovery of potent siRNA-containing lipid nanoparticles enabled by controlled microfluidic formulation,” J. Am. Chem. Soc. 134(16):6948-51 (2012)). Exemplary micromixers include Slit Interdigital Microstructured Mixer (SIMM-V2) or a Standard Slit Interdigital Micro Mixer (SSIMM) or Caterpillar (CPMM) or Impinging-jet (IJMM,) from the Institut fur Mikrotechnik Mainz GmbH, Mainz Germany. In some embodiments, methods of making LNP using SHM further comprise mixing at least two input streams wherein mixing occurs by microstructure-induced chaotic advection (MICA). According to this method, fluid streams flow through channels present in a herringbone pattern causing rotational flow and folding the fluids around each other. This method can also comprise a surface for fluid mixing wherein the surface changes orientations during fluid cycling. Methods of generating LNPs using SHM include those disclosed in U.S. Pub. Nos. US20040262223 and US20120276209.

In some embodiments, the polynucleotides described herein can be formulated in lipid nanoparticles using microfluidic technology (see Whitesides, George M., “The Origins and the Future of Microfluidics,” Nature 442: 368-373 (2006); and Abraham et al., “Chaotic Mixer for Microchannels,” Science 295: 647-651 (2002)). In some embodiments, the polynucleotides can be formulated in lipid nanoparticles using a micromixer chip such as, but not limited to, those from Harvard Apparatus (Holliston, MA) or Dolomite Microfluidics (Royston, UK). A micromixer chip can be used for rapid mixing of two or more fluid streams with a split and recombine mechanism.

i. Ionizable Lipids

The lipid nanoparticles described herein comprise ionizable lipids, including purified IALs, as discussed in the corresponding text above.

ii. Polynucleotides

Polynucleotides for use in accordance with the present disclosure include, but are not limited to, one or more of DNA, RNA including messenger RNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc., described in detail herein.

In some embodiments, the polynucleotide is RNA. In some embodiments, the polynucleotide is mRNA.

In some embodiments, the polynucleotide (e.g., a RNA, e.g., an mRNA) of the lipid nanoparticle comprises from about 900 to about 100,000 nucleotides (e.g., from 900 to 1,000, from 900 to 1,100, from 900 to 1,200, from 900 to 1,300, from 900 to 1,400, from 900 to 1,500, from 1,000 to 1,100, from 1,000 to 1,100, from 1,000 to 1,200, from 1,000 to 1,300, from 1,000 to 1,400, from 1,000 to 1,500, from 1,187 to 1,200, from 1,187 to 1,400, from 1,187 to 1,600, from 1,187 to 1,800, from 1,187 to 2,000, from 1,187 to 3,000, from 1,187 to 5,000, from 1,187 to 7,000, from 1,187 to 10,000, from 1,187 to 25,000, from 1,187 to 50,000, from 1,187 to 70,000, or from 1,187 to 100,000 nucleotides).

In some embodiments, the polynucleotide (e.g., a RNA, e.g., an mRNA) of the lipid nanoparticle comprises a nucleotide sequence (e.g., an open reading frame (ORF)) encoding a polypeptide, wherein the length of the nucleotide sequence (e.g., an ORF) is at least 500 nucleotides in length, e.g., at least about 500, 600, 700, 80, 900, 1,000, 1,050, 1,100, 1,187, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 7,000, 8,000, 9,000, 10,000, 20,000, 30,000, 40,000, 50,000, 60,000, 70,000, 80,000, or 90,000 nucleotides. In some embodiments, the length is up to and including 100,000 nucleotides.

In some embodiments, the polynucleotide of the composition comprises a nucleotide sequence (e.g., an ORF) encoding a polypeptide is DNA.

In some embodiments, the polynucleotide of the composition is RNA. In some embodiments, the polynucleotide is, or functions as, an mRNA. In some embodiments, the mRNA comprises a nucleotide sequence (e.g., an ORF) that encodes at least one polypeptide, and is capable of being translated to produce the encoded polypeptide in vitro, in vivo, in situ or ex vivo.

In some embodiments, the polynucleotide (e.g., a RNA, e.g., an mRNA) of the lipid nanoparticle comprises a nucleotide sequence (e.g., an ORF) encoding a polypeptide and further comprises at least one nucleic acid sequence that is noncoding, e.g., a microRNA binding site, e.g., a miRNA binding site that binds to miR-142 and/or a miRNA binding site that binds to miR-126.

In some embodiments, the polynucleotide (e.g., a RNA, e.g., an mRNA) of the lipid nanoparticle comprises a 5′-UTR and a 3′UTR.

In some embodiments, the polynucleotide (e.g., a RNA, e.g., an mRNA) of the lipid nanoparticle comprises a 5′ terminal cap. Non-limiting examples of 5′ terminal caps include Cap0, Cap1, ARCA, inosine, N1-methyl-guanosine, 2′-fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

In some embodiments, the polynucleotide (e.g., a RNA, e.g., an mRNA) of the lipid nanoparticle comprises a poly-A-tail. In some embodiments, the polyA tail is about 100 nucleotides in length. In some instances, the poly A tail is 100 nucleotides in length. In some instances, the poly A tail is 50-150, 75-150, 85-150, 90-150, 90-120, 90-130, or 90-150 nucleotides in length.

The polynucleotides (e.g., a RNA, e.g., an mRNA) can also comprise nucleotide sequences that encode additional features that facilitate trafficking of the encoded polypeptides to therapeutically relevant sites. One such feature that aids in protein trafficking is the signal sequence, or targeting sequence. The peptides encoded by these signal sequences are known by a variety of names, including targeting peptides, transit peptides, and signal peptides.

In some embodiments, the “signal sequence” or “signal peptide” is a polynucleotide or polypeptide, respectively, which is from about 30-210, e.g., about 45-80 or 15-60 nucleotides (e.g., about 20, 30, 40, 50, 60, or 70 amino acids) in length that, optionally, is incorporated at the 5′ (or N-terminus) of the coding region or the polypeptide, respectively. Addition of these sequences results in trafficking the encoded polypeptide to a desired site, such as the endoplasmic reticulum or the mitochondria through one or more targeting pathways. Some signal peptides are cleaved from the protein, for example by a signal peptidase after the proteins are transported to the desired site.

In some embodiments, the polynucleotide (e.g., a RNA, e.g., an mRNA) is sequence optimized. In some embodiments, the polynucleotide (e.g., a RNA, e.g., an mRNA) comprises a 5′ cap, a 5′-UTR, a nucleotide sequence (e.g., an ORF, e.g., a sequence optimized ORF) encoding a polypeptide, a 3′-UTR, and a polyA tail, or any combination thereof, the 5′ UTR or 3′ UTR optionally comprising at least one microRNA binding site.

A sequence-optimized nucleotide sequence, e.g., a codon-optimized mRNA sequence encoding a polypeptide, is a sequence comprising at least one synonymous nucleobase substitution with respect to a reference sequence (e.g., a wild type nucleotide sequence encoding the polypeptide).

A sequence-optimized nucleotide sequence can be partially or completely different in sequence from the reference sequence. For example, a reference sequence encoding polyserine uniformly encoded by UCU codons can be sequence-optimized by having 100% of its nucleobases substituted (for each codon, U in position 1 replaced by A, C in position 2 replaced by G, and U in position 3 replaced by C) to yield a sequence encoding polyserine which would be uniformly encoded by AGC codons. The percentage of sequence identity obtained from a global pairwise alignment between the reference polyserine nucleic acid sequence and the sequence-optimized polyserine nucleic acid sequence would be 0%. However, the protein products from both sequences would be 100% identical.

Some sequence optimization (also sometimes referred to codon optimization) methods are known in the art (and discussed in more detail below) and can be useful to achieve one or more desired results. These results can include, e.g., matching codon frequencies in certain tissue targets and/or host organisms to ensure proper folding; biasing G/C content to increase mRNA stability or reduce secondary structures; minimizing tandem repeat codons or base runs that can impair gene construction or expression; customizing transcriptional and translational control regions; inserting or removing protein trafficking sequences; removing/adding post translation modification sites in an encoded protein (e.g., glycosylation sites); adding, removing or shuffling protein domains; inserting or deleting restriction sites; modifying ribosome binding sites and mRNA degradation sites; adjusting translational rates to allow the various domains of the protein to fold properly; and/or reducing or eliminating problem secondary structures within the polynucleotide. Sequence optimization tools, algorithms and services are known in the art, non-limiting examples include services from GeneArt (Life Technologies), DNA2.0 (Menlo Park CA) and/or are proprietary methods.

In some embodiments, the sequence optimization method is multiparametric and comprises one, two, three, four, or more methods disclosed herein and/or other optimization methods known in the art.

In some embodiments, a polynucleotide (e.g., a RNA, e.g., an mRNA) of the composition comprises a sequence-optimized nucleotide sequence (e.g., an ORF) encoding a polypeptide, wherein the polypeptide encoded by the sequence-optimized nucleotide sequence has improved properties (e.g., compared to the polypeptide as encoded by a reference nucleotide sequence that is not sequence optimized), e.g., improved properties related to expression efficacy after administration in vivo. Such properties include, but are not limited to, improving nucleic acid stability (e.g., mRNA stability), increasing translation efficacy in the target tissue, reducing the number of truncated proteins expressed, improving the folding or prevent misfolding of the expressed proteins, reducing toxicity of the expressed products, reducing cell death caused by the expressed products, increasing and/or decreasing protein aggregation.

In some embodiments, the sequence-optimized nucleotide sequence (e.g., an ORF) is codon optimized for expression in human subjects, having structural and/or chemical features that avoid one or more of the problems in the art, for example, features which are useful for optimizing formulation and delivery of nucleic acid-based therapeutics while retaining structural and functional integrity; overcoming a threshold of expression; improving expression rates; half-life and/or protein concentrations; optimizing protein localization; and avoiding deleterious bio-responses such as the immune response and/or degradation pathways.

Methods for optimizing codon usage are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or are proprietary methods. In some embodiments, the open reading frame (ORF) sequence is optimized using optimization algorithms.

Features, which can be considered beneficial in some embodiments, can be encoded by or within regions of the polynucleotide and such regions can be upstream (5′) to, downstream (3′) to, or within the region that encodes the polypeptide. These regions can be incorporated into the polynucleotide before and/or after sequence-optimization of the protein encoding region or open reading frame (ORF). Examples of such features include, but are not limited to, untranslated regions (UTRs), microRNA sequences, Kozak sequences, oligo(dT) sequences, poly-A tail, and detectable tags and can include multiple cloning sites that can have XbaI recognition.

In some embodiments, the polynucleotide comprises a 5′ UTR, a 3′ UTR and/or a microRNA binding site. In some embodiments, the polynucleotide comprises two or more 5′ UTRs and/or 3′ UTRs, which can be the same or different sequences. In some embodiments, the polynucleotide comprises two or more microRNA binding sites, which can be the same or different sequences. Any portion of the 5′ UTR, 3′ UTR, and/or microRNA binding site, including none, can be sequence-optimized and can independently contain one or more different structural or chemical modifications, before and/or after sequence optimization.

In some embodiments, the polynucleotides of the compositions are modified. The modified polynucleotides can be chemically modified and/or structurally modified. When the polynucleotides of the compositions are chemically and/or structurally modified the polynucleotides can be referred to as “modified polynucleotides.”

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside including a phosphate group. Modified nucleotides can be synthesized by any useful method, such as, for example, chemically, enzymatically, or recombinantly, to include one or more modified or non-natural nucleosides. Polynucleotides can comprise a region or regions of linked nucleosides. Such regions can have variable backbone linkages. The linkages can be standard phosphodiester linkages, in which case the polynucleotides would comprise regions of nucleotides.

The modified polynucleotides disclosed herein can comprise various distinct modifications. In some embodiments, the modified polynucleotides contain one, two, or more (optionally different) nucleoside or nucleotide modifications. In some embodiments, a modified polynucleotide, introduced to a cell can exhibit one or more desirable properties, e.g., improved protein expression, reduced immunogenicity, or reduced degradation in the cell, as compared to an unmodified polynucleotide.

In some embodiments, a polynucleotide of the lipid nanoparticles are structurally modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” can be chemically modified to “AT-5meC-G”. The same polynucleotide can be structurally modified from “ATCG” to “ATCCCG”. Here, the dinucleotide “CC” has been inserted, resulting in a structural modification to the polynucleotide.

Therapeutic lipid nanoparticles comprise, in some embodiments, at least one nucleic acid (e.g., RNA), wherein the nucleic acid comprises nucleotides and/or nucleosides that can be standard (unmodified) or modified as is known in the art. In some embodiments, nucleotides and nucleosides comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleoside is one as is generally known or recognized in the art. Non-limiting examples of such naturally occurring modified nucleotides and nucleosides can be found, inter alia, in the MODOMICS database.

In some embodiments, a non-naturally occurring modified nucleotide or nucleoside is one as is generally known or recognized in the art. Non-limiting examples of such non-naturally occurring modified nucleotides and nucleosides can be found, inter alia, in published International Patent Application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367.

In some embodiments, at least one RNA (e.g., mRNA) is not chemically modified and comprises the standard ribonucleotides consisting of adenosine, guanosine, cytosine and uridine. In some embodiments, nucleotides and nucleosides comprise standard nucleoside residues such as those present in transcribed RNA (e.g. A, G, C, or U). In some embodiments, nucleotides and nucleosides comprise standard deoxyribonucleosides such as those present in DNA (e.g. dA, dG, dC, or dT).

Hence, nucleic acids (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminus of a chain or anywhere else in the chain. Any of the regions of a nucleic acid may be chemically modified.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as in those nucleic acids having at least one chemical modification. One example of such non-standard base pairing is the base pairing between the modified nucleotide inosine and adenine, cytosine or uracil. Any combination of base/sugar or linker may be incorporated into nucleic acids.

In some embodiments, modified nucleobases in nucleic acids (e.g., RNA nucleic acids, such as mRNA nucleic acids) comprise N1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with N1-methyl-pseudouridine, meaning that all uridine residues in the mRNA sequence are replaced with N1-methyl-pseudouridine. Similarly, a nucleic acid can be uniformly modified for any type of nucleoside residue present in the sequence by replacement with a modified residue such as those set forth above.

The nucleic acids may be partially or fully modified along the entire length of the molecule. For example, one or more or all or a given type of nucleotide (e.g., purine or pyrimidine, or any one or more or all of A, G, U, C) may be uniformly modified in a nucleic acid, or in a predetermined sequence region thereof (e.g., in the mRNA including or excluding the polyA tail). In some embodiments, all nucleotides X in a nucleic acid (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

The polynucleotides of the lipid nanoparticles can be generated using components, compositions, and methods as are generally known in the art, see, e.g., International Patent Application Publication Nos. WO 2015/051173, WO 2017/049286, WO 2016/100812, WO 2016/014846, WO 2016/011226, WO 2016/011222, WO 2016/011306, WO 2015/196128, WO 2013/151736, WO 2013/151672, WO 2013/151671, WO 2013/151670, WO 2013/151669, WO 2013/151668, WO 2013/151666, WO 2013/151667, WO 2013/151665, WO 2013/151664, WO 2013/151663, WO 2013/151736, WO 2013/151668, WO 2013/151666, WO 2013/151665, WO 2013/151670, WO 2013/151672, WO 2015/089511, WO 2015/051173, WO 2015/051169.

In some aspects, a polynucleotide (e.g., a RNA, e.g., an mRNA) disclosed herein can be constructed using in vitro transcription (IVT). In other aspects, a polynucleotide (e.g., a RNA, e.g., an mRNA) disclosed herein can be constructed by chemical synthesis using an oligonucleotide synthesizer.

In other aspects, a polynucleotide (e.g., a RNA, e.g., an mRNA) disclosed herein is made by using a host cell. In certain aspects, a polynucleotide (e.g., a RNA, e.g., an mRNA) disclosed herein is made by one or more combination of the IVT, chemical synthesis, host cell expression, or any other methods known in the art.

Naturally occurring nucleosides, non-naturally occurring nucleosides, or combinations thereof, can totally or partially naturally replace occurring nucleosides present in the candidate nucleotide sequence and can be incorporated into a sequence-optimized nucleotide sequence (e.g., a RNA, e.g., an mRNA). The resultant polynucleotides, e.g., mRNAs, can then be examined for their ability to produce protein and/or produce a therapeutic outcome.

### (a) Purification of Polynucleotides

The polynucleotides can be purified prior to their inclusion in the lipid nanoparticles. Purification of the polynucleotides described herein can include, but is not limited to, polynucleotide clean-up, quality assurance and quality control. Clean-up can be performed by methods known in the arts such as, but not limited to, AGENCOURT® beads (Beckman Coulter Genomics, Danvers, MA), poly-T beads, LNATM oligo-T capture probes (EXIQON® Inc., Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC).

In some embodiments, purification of a polynucleotide removes impurities that can reduce or remove an unwanted immune response, e.g., reducing cytokine activity.

In some embodiments, the polynucleotide is purified prior to inclusion in a lipid nanoparticle using column chromatography (e.g., strong anion exchange HPLC, weak anion exchange HPLC, reverse phase HPLC (RP-HPLC), and hydrophobic interaction HPLC (HIC-HPLC), or (LCMS)).

In some embodiments, the purified polynucleotide is at least about 80% pure, at least about 85% pure, at least about 90% pure, at least about 95% pure, at least about 96% pure, at least about 97% pure, at least about 98% pure, at least about 99% pure, or about 100% pure prior to inclusion in a lipid nanoparticle.

A quality assurance and/or quality control check can be conducted using methods such as, but not limited to, gel electrophoresis, UV absorbance, or analytical HPLC. In another embodiment, the polynucleotide can be sequenced by methods including, but not limited to reverse-transcriptase-PCR.

### (b) Quantification of Polynucleotides

In some embodiments, the polynucleotides, their expression products, as well as degradation products and metabolites can be quantified according to methods known in the art.

In some embodiments, the polynucleotide can be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified polynucleotide can be analyzed in order to determine if the polynucleotide can be of proper size, or to check that no degradation of the polynucleotide has occurred. Degradation of the polynucleotide can be checked by methods (such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE), capillary gel electrophoresis (CGE)); and UPLC (e.g., RP-UPLC).

iii. Other Lipid Nanoparticle Components

The lipid composition of a lipid nanoparticle disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof). A polymer can be included in and/or used to encapsulate or partially encapsulate a composition disclosed herein (e.g., an LNP composition). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

The LNP can further contain a phosphate conjugate. The phosphate conjugate can increase in vivo circulation times and/or increase the targeted delivery of the nanoparticle. Phosphate conjugates can be made by the methods described in, e.g., Intl. Pub. No. WO2013033438 or U.S. Pub. No. US20130196948. The LNP can also contain a polymer conjugate (e.g., a water soluble conjugate) as described in, e.g., U.S. Pub. Nos. US20130059360, US20130196948, and US20130072709.

The LNPs can comprise a conjugate to enhance the delivery of nanoparticles in a subject. Further, the conjugate can inhibit phagocytic clearance of the nanoparticles in a subject. In some embodiments, the conjugate can be a “self” peptide designed from the human membrane protein CD47 (e.g., the “self” particles described by Rodriguez et al, Science 2013 339, 971-975). As shown by Rodriguez et al. the self peptides delayed macrophage-mediated clearance of nanoparticles which enhanced delivery of the nanoparticles.

The LNPs can comprise a carbohydrate carrier. As a non-limiting example, the carbohydrate carrier can include, but is not limited to, an anhydride-modified phytoglycogen or glycogen-type material, phytoglycogen octenyl succinate, phytoglycogen beta-dextrin, anhydride-modified phytoglycogen beta-dextrin (e.g., Intl. Pub. No. WO2012109121).

The LNPs can be coated with a surfactant or polymer to improve the delivery of the particle. In some embodiments, the LNP can be coated with a hydrophilic coating such as, but not limited to, PEG coatings and/or coatings that have a neutral surface charge as described in U.S. Pub. No. US20130183244.

The LNPs can be engineered to alter the surface properties of particles so that the lipid nanoparticles can penetrate the mucosal barrier as described in U.S. Pat. No. 8,241,670 or Intl. Pub. No. WO2013110028.

The LNPs engineered to penetrate mucus can comprise a polymeric material (e.g., a polymeric core) and/or a polymer-vitamin conjugate and/or a tri-block co-polymer. The polymeric material can include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates.

LNPs engineered to penetrate mucus can also include surface altering agents such as, but not limited to, polynucleotides, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin P4 dornase alfa, neltenexine, erdosteine) and various DNases including rhDNase.

In some embodiments, the mucus penetrating LNP can be a hypotonic formulation comprising a mucosal penetration enhancing coating. The formulation can be hypotonic for the epithelium to which it is being delivered. Non-limiting examples of hypotonic formulations can be found in, e.g., Intl. Pub. No. WO2013110028.

### (a) Other Lipids

### (i) Phospholipids

The lipid composition of a lipid nanoparticle disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In certain embodiments, a phospholipid useful or potentially useful in the present disclosure is an analog or variant of DSPC. In certain embodiments, a phospholipid useful or potentially useful in the present disclosure is a compound of Formula (IV):

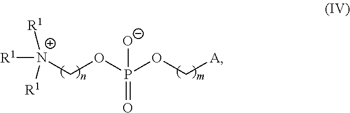

or a salt thereof, wherein
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

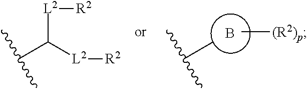

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or —NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, —OC(O)N(R^{N}), NR^{N}C(O)O, C(O)S, SC(O), C(═NR^{N}), C(═NR^{N})N(R^{N}NR^{N}C(═NR^{N}), —NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), —OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}),
or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

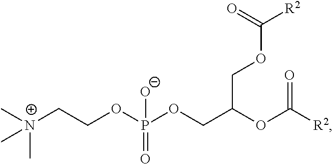

wherein each instance of R² is independently unsubstituted alkyl, unsubstituted alkenyl, or unsubstituted alkynyl.

In some embodiments, the phospholipids may be one or more of the phospholipids described in PCT/US2018/037922 (published as WO 2018232357).

### A) Phospholipid Head Modifications

In certain embodiments, a phospholipid useful or potentially useful in the present disclosure comprises a modified phospholipid head (e.g., a modified choline group). In certain embodiments, a phospholipid with a modified head is DSPC, or analog thereof, with a modified quaternary amine. For example, in embodiments of Formula (IV), at least one of R¹ is not methyl. In certain embodiments, at least one of R¹ is not hydrogen or methyl. In certain embodiments, the compound of Formula (IV) is of one of the following formulae:

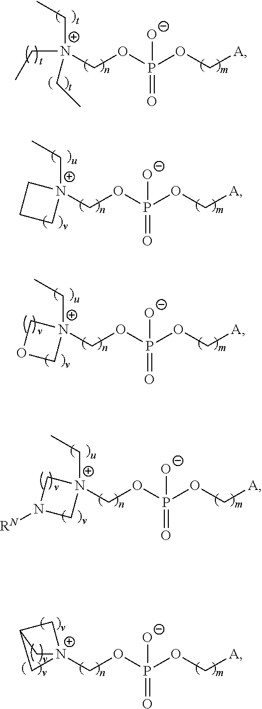

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, a compound of Formula (IV) is of Formula (IV-a):

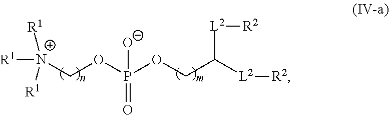

or a salt thereof.

In certain embodiments, a phospholipid useful or potentially useful in the present disclosure comprises a cyclic moiety in place of the glyceride moiety. In certain embodiments, a phospholipid useful in the present disclosure is DSPC, or analog thereof, with a cyclic moiety in place of the glyceride moiety. In certain embodiments, the compound of Formula (IV) is of Formula (IV-b):

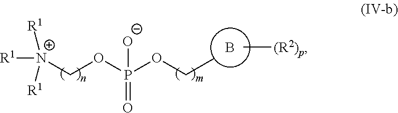

or a salt thereof.

### B) Phospholipid Tail Modifications

In certain embodiments, a phospholipid useful or potentially useful in the present disclosure comprises a modified tail. In certain embodiments, a phospholipid useful or potentially useful in the present disclosure is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IV) is of Formula (IV-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), —NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, —N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O.

In certain embodiments, the compound of Formula (IV) is of Formula (IV-c):

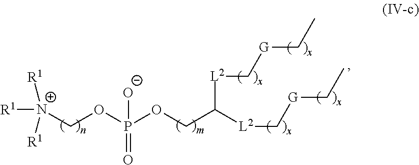

or a salt thereof, wherein:
each x is independently an integer between 0-30, inclusive; and each instance is G is independently selected from the group consisting of optionally substituted carbocyclylene, optionally substituted heterocyclylene, optionally substituted arylene, optionally substituted heteroarylene, N(R^{N}), 0, S, C(O), C(O)N(R^{N}), NR^{N}C(O), NR^{N}C(O)N(R^{N}) C(O)O, OC(O), OC(O)O, OC(O)N(R^{N}), NR^{N}C(O)O, C(O)S, SC(O), C(═NR^{N}), C(═NR^{N})N(R^{N}), —NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), —OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O. Each possibility represents separate embodiments.

In certain embodiments, a phospholipid useful or potentially useful in the present disclosure comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present disclosure is a compound of Formula (IV), wherein n is 1, 3, 4, 5, 6, 7, 8, 9, or 10. For example, in certain embodiments, a compound of Formula (IV) is of one of the following formulae:

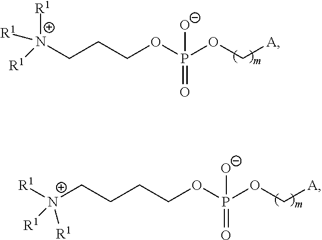

or a salt thereof.

### (ii) Alternative Lipids

In certain embodiments, a phospholipid useful or potentially useful in the present disclosure comprises a modified phosphocholine moiety, wherein the alkyl chain linking the quaternary amine to the phosphoryl group is not ethylene (e.g., n is not 2). Therefore, in certain embodiments, a phospholipid useful.

In certain embodiments, an alternative lipid is used in place of a phospholipid.

In certain embodiments, an alternative lipid is oleic acid.

In certain embodiments, the alternative lipid is one of the following:

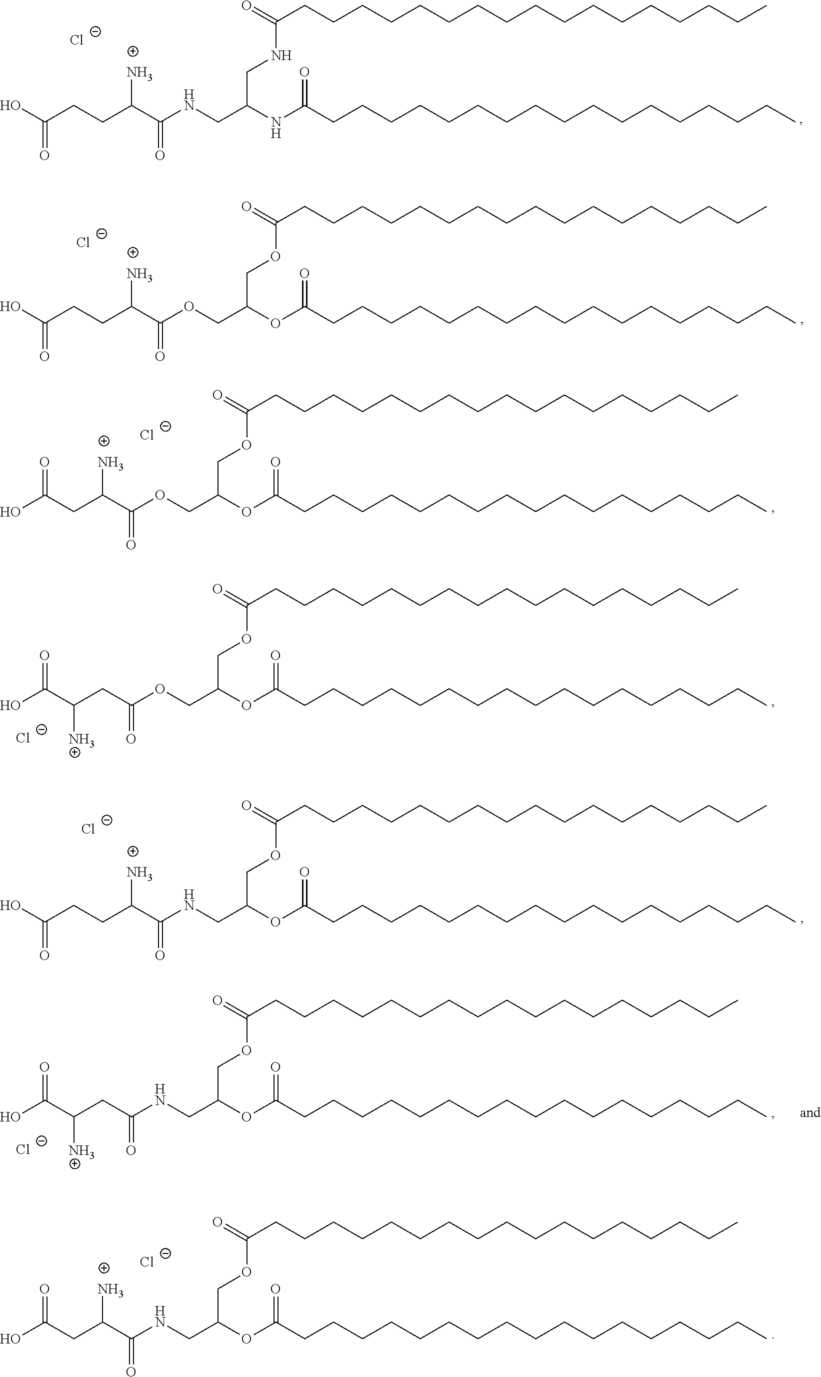

(iii) Structural Lipids

The lipid composition of a lipid nanoparticle disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in PCT/US2018/037922 (published as WO2018232357).

In some embodiments, the structural lipid is cholesterol. In some embodiments, the amount of the structural lipids (e.g., cholesterol) in the lipid composition ranges from about 20 mol % to about 60 mol %.

### (iv) Polyethylene Glycol (PEG)-Lipids

The lipid composition of a lipid nanoparticle disclosed herein can comprise one or more polyethylene glycol (PEG) lipids.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In some embodiments, the PEG-lipid is PEG₂ₖ-DMG.

In some embodiments, the lipid nanoparticles described herein can comprise a PEG lipid which is a non-diffusible PEG. Non-limiting examples of non-diffusible PEGs include PEG-DSG and PEG-DSPE. PEG-lipids are known in the art, such as those described in U.S. Pat. No. 8,158,601 and International Publ. No. WO 2015/130584 A2.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents.”

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

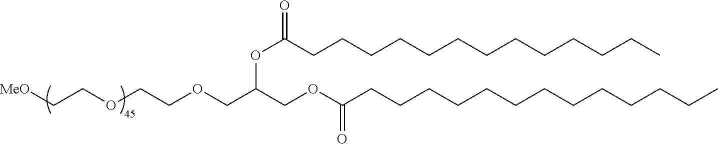

In some embodiments, PEG lipids useful in the present disclosure can be PEGylated lipids described in International Publication No. WO2012099755. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents separate embodiments.

In certain embodiments, a PEG lipid useful in the present disclosure is a compound of Formula (V). Provided herein are compounds of Formula (V):

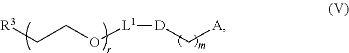

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, —OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

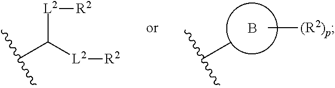

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or —NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, —OC(O)N(R^{N}), NR^{N}C(O)O, C(O)S, SC(O), C(═NR^{N}), C(═NR^{N})N(R^{N}), NR^{N}C(═NR^{N}), —NR^{N}C(═NR^{N})N(R^{N}), C(S), C(S)N(R^{N}), NR^{N}C(S), NR^{N}C(S)N(R^{N}) S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), —N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (V) is a PEG-OH lipid (i.e., R³ is —OR^{O}, and R^{O} is hydrogen). In certain embodiments, the compound of Formula (V) is of Formula (V-OH):

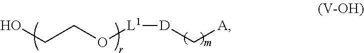

or a salt thereof.

In certain embodiments, a PEG lipid useful in the present disclosure is a PEGylated fatty acid. In certain embodiments, a PEG lipid useful in the present disclosure is a compound of Formula (VI). Provided herein are compounds of Formula (VI):

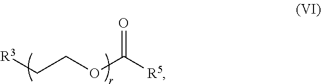

or a salts thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}) NR^{N}C(═NR^{N})N(R^{N}) C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (VI) is of Formula (VI—OH):

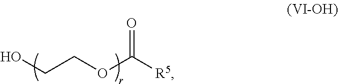

or a salt thereof. In some embodiments, r is 45.

In yet other embodiments the compound of Formula (VI) is:

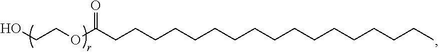

or a salt thereof.

In some embodiments, the compound of Formula (VI) is

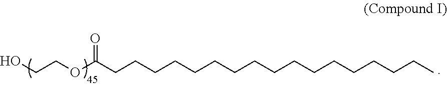

In some aspects, the lipid composition of the pharmaceutical compositions disclosed herein does not comprise a PEG-lipid.

In some embodiments, the PEG-lipids may be one or more of the PEG lipids described in PCT/US2018/037922 (published as WO 2018232357).

In some embodiments, a PEG lipid comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a LNP comprises an ionizable cationic lipid of any of Formula I, II or III, a phospholipid comprising DSPC, a structural lipid, and a PEG lipid comprising PEG-DMG.

In some embodiments, a LNP comprises an ionizable cationic lipid of any of Formula I, II or III, a phospholipid comprising DSPC, a structural lipid, and a PEG lipid comprising a compound having Formula VI.

In some embodiments, a LNP comprises an ionizable cationic lipid of any of Formula I, II or III, a phospholipid comprising a compound having Formula IV, a structural lipid, and the PEG lipid comprising a compound having Formula V.

In some embodiments, a LNP comprises an ionizable cationic lipid of any of Formula I, II or III, a phospholipid comprising a compound having Formula IV, a structural lipid, and the PEG lipid comprising a compound having Formula VI.

In some embodiments, a LNP comprises an ionizable cationic lipid of any of Formula I, II or III, a phospholipid comprising a compound having Formula IV, a structural lipid, and a PEG lipid comprising a compound having Formula V or VI.

In some embodiments, a LNP comprises an ionizable cationic lipid of

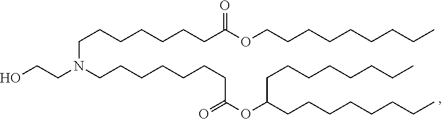 [SMILES: CCCCCCCCCOC(=O)CCCCCCCN(CCO)CCCCCCCC(=O)OC(CCCCCCCC)CCCCCCCC]

and a PEG lipid comprising Formula VI.

In some embodiments, a LNP comprises an ionizable cationic lipid of

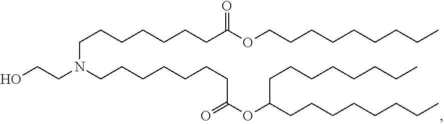 [SMILES: CCCCCCCCCOC(=O)CCCCCCCN(CCO)CCCCCCCC(=O)OC(CCCCCCCC)CCCCCCCC]

and an alternative lipid comprising oleic acid.

In some embodiments, a LNP comprises an ionizable cationic lipid of

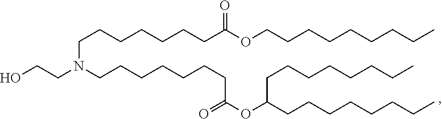 [SMILES: CCCCCCCCCOC(=O)CCCCCCCN(CCO)CCCCCCCC(=O)OC(CCCCCCCC)CCCCCCCC]

an alternative lipid comprising oleic acid, a structural lipid comprising cholesterol, and a PEG lipid comprising a compound having Formula VI.

In some embodiments, a LNP comprises an ionizable cationic lipid of

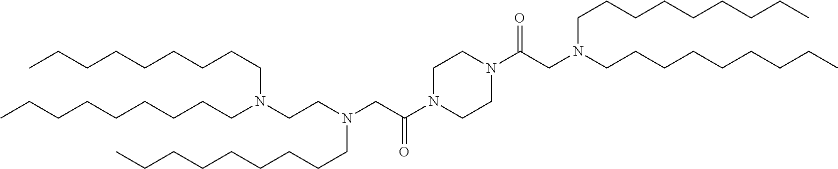 [SMILES: CCCCCCCCCN(CCCCCCCCC)CCN(CCCCCCCCC)CC(=O)N1CCN(C(=O)CN(CCCCCCCCC)CCCCCCCCC)CC1]

a phospholipid comprising DOPE, a structural lipid comprising cholesterol, and a PEG lipid comprising a compound having Formula VI.

In some embodiments, a LNP comprises an ionizable cationic lipid of a phospholipid comprising DOPE, a structural lipid comprising cholesterol, and a PEG lipid comprising a compound having Formula VII.

In some embodiments, a LNP comprises an N:P ratio of from about 2:1 to about 30:1. In some embodiments, a LNP comprises an N:P ratio of about 6:1. In some embodiments, a LNP comprises an N:P ratio of about 3:1. In some embodiments, a LNP comprises a wt/wt ratio of the ionizable cationic lipid component to the RNA of from about 10:1 to about 100:1. In some embodiments, a LNP comprises a wt/wt ratio of the ionizable cationic lipid component to the RNA of about 20:1. In some embodiments, a LNP comprises a wt/wt ratio of the ionizable cationic lipid component to the RNA of about 10:1. the disclosure has a mean diameter from about 50 nm to about 150 nm. In some embodiments, a LNP has a mean diameter from about 70 nm to about 120 nm.

### (b) Liposomes

The ionizable lipids disclosed herein (e.g., purified IALs) may be used to prepare liposomes. Liposomes are artificially-prepared vesicles that can primarily be composed of a lipid bilayer and can be used as a delivery vehicle for the administration of pharmaceutical formulations. Liposomes can be of different sizes. A multilamellar vesicle (MLV) can be hundreds of nanometers in diameter, and can contain a series of concentric bilayers separated by narrow aqueous compartments. A small unicellular vesicle (SUV) can be smaller than 50 nm in diameter, and a large unilamellar vesicle (LUV) can be between 50 and 500 nm in diameter. Liposome design can include, but is not limited to, opsonins or ligands to improve the attachment of liposomes to unhealthy tissue or to activate events such as, but not limited to, endocytosis. Liposomes can contain a low or a high pH value in order to improve the delivery of the pharmaceutical formulations.

The formation of liposomes can depend on the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimal size, polydispersity and the shelf-life of the vesicles for the intended application, and the batch-to-batch reproducibility and scale up production of safe and efficient liposomal products, etc.

As a non-limiting example, liposomes such as synthetic membrane vesicles can be prepared by the methods, apparatus and devices described in U.S. Pub. Nos. US20130177638, US20130177637, US20130177636, US20130177635, US20130177634, US20130177633, US20130183375, US20130183373, and US20130183372. In some embodiments, the polynucleotides described herein can be encapsulated by the liposome and/or it can be contained in an aqueous core that can then be encapsulated by the liposome as described in, e.g., Intl. Pub. Nos. WO2012031046, WO2012031043, WO2012030901, WO2012006378, and WO2013086526; and U.S. Pub. Nos. US20130189351, US20130195969 and US20130202684.

### (c) Lipoplexes

In some embodiments, the ionizable lipids (e.g., IALs) and polynucleotides described herein are formulated as lipoplexes, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, MA), and polyethylenimine (PEI) or protamine-based targeted and non-targeted delivery of nucleic acids. Exemplary lipoplexes are disclosed in: Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; and deFougerolles Hum Gene Ther. 2008 19:125-132.

### 8. Pharmaceutical Compositions

The compositions comprising delivery agents (e.g., LNP compositions, liposomes, lipoplexes, etc.) may also be formulated as pharmaceutical compositions. Pharmaceutical compositions can optionally comprise one or more additional active substances, e.g., therapeutically and/or prophylactically active substances. Pharmaceutical compositions can be sterile and/or pyrogen-free. General considerations in the formulation and/or manufacture of pharmaceutical agents can be found, for example, in Remington: The Science and Practice of Pharmacy 21st ed., Lippincott Williams & Wilkins, 2005. In some embodiments, compositions are administered to humans, human patients or subjects. The phrase “active ingredient” generally refers to polynucleotides to be delivered as described herein.

Formulations and pharmaceutical compositions described herein can be prepared by any method known or hereafter developed in the art of pharmacology. In general, such preparatory methods include the step of associating the active ingredient with an excipient and/or one or more other accessory ingredients, and then, if necessary and/or desirable, dividing, shaping and/or packaging the product into a desired single- or multi-dose unit.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient that would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure can vary, depending upon the identity, size, and/or condition of the subject being treated and further depending upon the route by which the composition is to be administered.

In some embodiments, the compositions and formulations described herein can contain at least one LNP. As a non-limiting example, the composition can contain 1, 2, 3, 4 or 5 LNPs. In some embodiments, the compositions described herein can comprise more than one type of LNP.

Although the descriptions of pharmaceutical compositions and formulations provided herein are principally directed to pharmaceutical compositions and formulations that are suitable for administration to humans, it will be understood by the skilled artisan that such compositions are generally suitable for administration to any other animal, e.g., to non-human animals, e.g. non-human mammals.

The present disclosure provides pharmaceutical formulations that comprise an LNP described herein. The LNPs described herein can be formulated using one or more excipients to: (1) increase stability; (2) increase cell transfection; (3) permit the sustained or delayed release (e.g., from a depot formulation of the polynucleotide); (4) alter the biodistribution (e.g., target the LNP to specific tissues or cell types); (5) increase the translation of encoded protein in vivo; and/or (6) alter the release profile of encoded protein in vivo.

A pharmaceutically acceptable excipient, as used herein, includes, but are not limited to, any and all solvents, dispersion media, or other liquid vehicles, dispersion or suspension aids, diluents, granulating and/or dispersing agents, surface active agents, isotonic agents, thickening or emulsifying agents, preservatives, binders, lubricants or oil, coloring, sweetening or flavoring agents, stabilizers, antioxidants, antimicrobial or antifungal agents, osmolality adjusting agents, pH adjusting agents, buffers, chelants, cyoprotectants, and/or bulking agents, as suited to the particular dosage form desired. Various excipients for formulating pharmaceutical compositions and techniques for preparing the composition are known in the art (see Remington: The Science and Practice of Pharmacy, 21st Edition, A. R. Gennaro (Lippincott, Williams & Wilkins, Baltimore, MD, 2006).

Exemplary diluents include, but are not limited to, calcium or sodium carbonate, calcium phosphate, calcium hydrogen phosphate, sodium phosphate, lactose, sucrose, cellulose, microcrystalline cellulose, kaolin, mannitol, sorbitol, etc., and/or combinations thereof.

Exemplary granulating and/or dispersing agents include, but are not limited to, starches, pregelatinized starches, or microcrystalline starch, alginic acid, guar gum, agar, poly(vinyl-pyrrolidone), (providone), cross-linked poly(vinyl-pyrrolidone) (crospovidone), cellulose, methylcellulose, carboxymethyl cellulose, cross-linked sodium carboxymethyl cellulose (croscarmellose), magnesium aluminum silicate (VEEGUM®), sodium lauryl sulfate, etc., and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), sorbitan fatty acid esters (e.g., polyoxyethylene sorbitan monooleate [TWEEN®80], sorbitan monopalmitate [SPAN®40], glyceryl monooleate, polyoxyethylene esters, polyethylene glycol fatty acid esters (e.g., CREMOPHOR®), polyoxyethylene ethers (e.g., polyoxyethylene lauryl ether [BRIJ®30]), PLUORINC®F 68, POLOXAMER®188, etc. and/or combinations thereof.

Exemplary binding agents include, but are not limited to, starch, gelatin, sugars (e.g., sucrose, glucose, dextrose, dextrin, molasses, lactose, lactitol, mannitol), amino acids (e.g., glycine), natural and synthetic gums (e.g., acacia, sodium alginate), ethylcellulose, hydroxyethylcellulose, hydroxypropyl methylcellulose, etc., and combinations thereof.

Exemplary antioxidants include, but are not limited to, alpha tocopherol, ascorbic acid, ascorbyl palmitate, benzyl alcohol, butylated hydroxyanisole, m-cresol, methionine, butylated hydroxytoluene, monothioglycerol, sodium or potassium metabisulfite, propionic acid, propyl gallate, sodium ascorbate, etc., and combinations thereof.

Exemplary chelating agents include, but are not limited to, ethylenediaminetetraacetic acid (EDTA), citric acid monohydrate, disodium edetate, fumaric acid, malic acid, phosphoric acid, sodium edetate, tartaric acid, trisodium edetate, etc., and combinations thereof.

Exemplary antimicrobial or antifungal agents include, but are not limited to, benzalkonium chloride, benzethonium chloride, methyl paraben, ethyl paraben, propyl paraben, butyl paraben, benzoic acid, hydroxybenzoic acid, potassium or sodium benzoate, potassium or sodium sorbate, sodium propionate, sorbic acid, etc., and combinations thereof.

Exemplary preservatives include, but are not limited to, vitamin A, vitamin C, vitamin E, beta-carotene, citric acid, ascorbic acid, butylated hydroxyanisol, ethylenediamine, sodium lauryl sulfate (SLS), sodium lauryl ether sulfate (SLES), etc., and combinations thereof.

In some embodiments, the pH of polynucleotide solutions is maintained between pH 5 and pH 8 to improve stability. Exemplary buffers to control pH can include, but are not limited to sodium phosphate, sodium citrate, sodium succinate, histidine (or histidine-HCl), sodium malate, sodium carbonate, etc., and/or combinations thereof.

Exemplary lubricating agents include, but are not limited to, magnesium stearate, calcium stearate, stearic acid, silica, talc, malt, hydrogenated vegetable oils, polyethylene glycol, sodium benzoate, sodium or magnesium lauryl sulfate, etc., and combinations thereof.

The pharmaceutical composition described here can contain a cryoprotectant to stabilize a polynucleotide described herein during freezing. Exemplary cryoprotectants include, but are not limited to mannitol, sucrose, trehalose, lactose, glycerol, dextrose, etc., and combinations thereof.

The pharmaceutical composition described here can contain a bulking agent in lyophilized polynucleotide formulations to yield a “pharmaceutically elegant” cake, stabilize the lyophilized polynucleotides during long term (e.g., 36 month) storage. Exemplary bulking agents can include, but are not limited to sucrose, trehalose, mannitol, glycine, lactose, raffinose, and combinations thereof.

The various concepts introduced above and discussed in greater detail below may be implemented in any of numerous ways, as the described concepts are not limited to any particular manner of implementation. Examples of specific implementations and applications are provided primarily for illustrative purposes.

### 9. Definitions

In order that the present disclosure can be more readily understood, certain terms are defined. As used in this application, except as otherwise expressly provided herein, each of the following terms shall have the meaning set forth below. Additional definitions are set forth throughout the application. Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

In this specification and the appended claims, the singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Wherever aspects are described herein with the language “comprising,” otherwise analogous aspects described in terms of “consisting of” and/or “consisting essentially of” are also provided.

Where used herein, numeric ranges should be understood to include the endpoints thereof, as well as each value in between the endpoints.

Nucleotides are referred to by their commonly accepted single-letter codes. Unless otherwise indicated, nucleic acids are written left to right in 5′ to 3′ orientation. Nucleobases are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

As used herein, the term “about” as used in connection with a numerical value denotes a degree of approximation, as would be understood to a person skilled in the art. Thus, the term “about” means that the numerical value so modified is not limited to the exact number set forth, but can vary to some extent based on the context in which it is used as would be understood by a person skilled in the art. Unless otherwise apparent from the context or convention in the art, “about” means up to plus or minus 10% of the particular term.

As used herein in connection with a numerical value, the term “approximately” means up to +/−5% of the number set forth.

As used herein, the term “activity” is a measure (which can be a qualitative or quantitative measure) of the functioning of a molecule. Thus, activity can be used to determine the presence or quantity of a functional peptide (e.g., enzyme) present in a sample. In some embodiments, activity is of a polypeptide (e.g., an enzyme) encoded by an mRNA. Enzyme activity and specific activity, both of which can be measured using techniques known in the art, are exemplary types of activities. Enzyme activity refers to an amount (e.g., moles) of a substrate converted (e.g., to a product) per unit time. As is generally known by one skilled in the art, enzymes are macromolecular biological catalysts that accelerate biochemical reactions. The molecule upon which an enzyme acts is called a substrate and the enzyme catalyzes the conversion of the substrates into different molecules known as products. In some embodiments, the activity of a polypeptide is the conversion of a substrate into a product. In some embodiments, the activity of a polypeptide is determined by detecting the presence or determining an amount of product formed by the polypeptide in a sample or a subject.

As used herein, “administering” (“or administered”) refers to delivering an agent or composition to a subject or patient or contacting an agent or composition with a cell. Methods of administration may be systemic or selected to target delivery (e.g., to specifically deliver) to a specific region or system of a body. For example, a polynucleotide or lipid nanoparticle composition can be administered or delivered to a subject by an intravenous, intramuscular, intradermal, or subcutaneous route.

As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

As used herein with respect to two or more moieties or physical materials, the term “associated with” means that the moieties are connected with or interact with one another, either directly or indirectly. For instance, two moieties can be directly connected (e.g., covalently, ionically, or via other molecular interactions) or connected via one or more additional moieties that serves as a linking agent, to form a structure that is sufficiently stable so that the moieties remain physically associated under the conditions in which the structure is used (e.g., physiological conditions). In some embodiments, two or more moieties or physical materials are associated with each other when they are conjugated, linked, attached, or tethered to each other.

As used herein, the term “base pair” refers to two nucleobases on opposite complementary nucleic acid strands that interact via the formation of specific hydrogen bonds. As used herein, the term “Watson-Crick base pairing”, used interchangeably with “complementary base pairing”, refers to a set of base pairing rules, wherein a purine always binds with a pyrimidine, e.g., in which nucleobase adenine (A) forms a complementary base pair with thymine (T) and guanine (G) forms a complementary base pair with cytosine (C) in DNA molecules. In RNA molecules, thymine is replaced by uracil (U), which, similar to thymine (T), forms a complementary base pair with adenine (A). The complementary base pairs are bound together by hydrogen bonds and the number of hydrogen bonds differs between base pairs. As in known in the art, guanine (G)-cytosine (C) base pairs are bound by three (3) hydrogen bonds and adenine (A)-thymine (T) or uracil (U) base pairs are bound by two (2) hydrogen bonds. Base pairing interactions that do not follow these rules can occur in natural, non-natural, and synthetic nucleic acids and are referred to herein as “non-Watson-Crick base pairing” or alternatively “non-complementary base pairing”.

Biocompatible: As used herein, the term “biocompatible” means compatible with living cells, tissues, organs or systems posing little to no risk of injury, toxicity or rejection by the immune system.

As used herein, the term “biomarker” refers to a biological molecule found in blood, other body fluids, or tissues that is a sign of a normal or abnormal process, of a condition or disease, or of a likelihood a subject will or will not respond to a particular treatment. A biomarker can include a substance that is detected and/or measured in a sample or subject as an indicator of an activity of a polypeptide (e.g., a “response biomarker”). For example, a biomarker can be a product formed by the activity of a polypeptide encoded by an mRNA. In some aspects, a biomarker is a receptor whose expression and/or activity changes in response to the activity of a polypeptide encoded by an mRNA. In some aspects, the activity of a polypeptide is characterized or determined by measuring the level of an appropriate biomarker in sample(s) taken from a subject. The term “level of a biomarker” can be qualified or quantified using mass, weight, and/or concentration of the biomarker. It will be understood by the skilled artisan that a sample may be subjected to a step of substance purification, precipitation, separation, e.g. centrifugation and/or HPLC, and subsequently subjected to a step of determining the level of the biomarker, e.g. using mass spectrometric analysis. In exemplary embodiments, LC-MS can be used as a means for determining the level of a biomarker.

As used herein, the term “comparing” or “compared to” refers to the identification of the similarity or dissimiliarty of a particular property or measurable characteristic (e.g., amount) in one item relative to the same property or measurable characteristic (e.g., amount) in another item. For instance, a comparison can be a mathematical comparison of two or more values, e.g., of the levels of the biomarker(s) present in a sample. A comparison can be based on individual values, mean values, or average values. Comparing or comparison to can be in the context, for example, of comparing to a reference value, e.g., as compared to a reference blood plasma, serum, red blood cells (RBC) and/or tissue (e.g., liver, kidney, heart) biomarker level, and/or a reference serum, blood plasma, tissue (e.g., liver, kidney, or heart), and/or urinary biomarker level, in a subject prior to treatment (e.g., prior to administration of a therapeutic agent) or in a normal or healthy subject.

As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., some physiological conditions. In some embodiments, two or more moieties may be conjugated by direct covalent chemical bonding. In other embodiments, two or more moieties may be conjugated by ionic bonding or hydrogen bonding. In some embodiments, the conjugation is reversible, e.g., the moieties can be physically separated, which can result in the release (e.g., under certain physiological conditions) of one or more of the moieties.

As used herein, the term “contacting” means establishing physical contact between two or more substances. Unless otherwise specified or dictated by the context in which it is used, “contacting” includes contacting that occurs in vivo, in vitro, or ex vivo. For example, contacting a solution or compound with a reagent means adding the reagent to the solution (such that it may react with compounds in the solution) or directly mixing the reagent with the compound such that the reagent and compound may react with one another. As another example, contacting a cell disposed within an organism with a substance (e.g., an agent or composition) may occur in vivo, such as resulting from administering the substance to the organism. For a cell present in vitro, contacting the cell with a substance may occur in vitro, such as by adding the substance to a substrate in which the cell is disposed, such as a culture medium. Unless otherwise specified or dictated by the context in which it is used, “contacting a cell” includes contacting more than one cell.

As used herein, the term “derivative” refers to a compound that is derived from or formed from a parent compound by reaction or association with another chemical compound. Thus, a “derivative” of a compound may be a substitution product, an addition product, an adduct, or a salt of the compound formed by reaction with another chemical compound.

As used herein, when referring to polypeptides, the term “domain” refers to a motif of a polypeptide having one or more identifiable structural or functional characteristics or properties (e.g., binding capacity, serving as a site for protein-protein interactions).

As used herein, the term “effective amount” of an agent is an amount sufficient to effect a beneficial or desired result, for example, an extent of a chemical reaction or a biological or clinical result. As such, an “effective amount” depends upon the context in which it is being applied. In some contexts, the term “effective amount” can be used interchangeably with “effective dose.”

As used herein, the term “ex vivo” refers to events that occur outside of an organism (e.g., animal, plant, or microbe or cell or tissue thereof). Ex vivo events can take place in an environment minimally altered from a natural (e.g., in vivo) environment.

As used herein, the term “encapsulate” means to enclose, surround, or encase. In some embodiments, a compound, polynucleotide (e.g., an mRNA), or other composition may be fully encapsulated, partially encapsulated, or substantially encapsulated. For example, in some embodiments, an mRNA may be encapsulated in a lipid nanoparticle.

As used herein, “endonuclease” refers to a cellular enzyme that cleaves the phosphodiester bond within a polynucleotide chain. An endonuclease differs from an exonuclease that cleaves terminal phosphodiester bonds of polynucleotides. In some embodiments, an endonuclease refers to an enzyme that cleaves a phosphodiester bond of DNA, of RNA or of both DNA and RNA. In some embodiments, an endonuclease refers to an enzyme that cleaves the phosphodiester bond within RNA. In some embodiments, an endonuclease cleaves a phosphodiester bond within an RNA to generate a 5′ RNA product and a 3′ RNA product, wherein the 5′ RNA product comprises a 3′ hydroxyl terminus and the 3′ RNA product comprises a 5′ phosphate group. In some embodiments, an endonuclease cleaves a phosphodiester bond within an RNA to generate a 5′ RNA product and a 3′ RNA product, wherein the 5′ RNA product comprises a 2′3′ cyclic phosphate and the 3′ RNA product comprises a 5′ hydroxyl group. In some embodiments, an endonuclease cleaves a phosphodiester bond non-specifically, wherein cleavage occurs at any site within an RNA regardless of the surrounding RNA sequence or structure. In some embodiments, an endonuclease cleaves a phosphodiester bond specifically, wherein cleavage occurs at specific sites within an RNA that is dependent upon the surrounding RNA sequence or structure.

As used herein, the term “enhanced delivery” means delivery of more agent to a target tissue compared to the level of delivery achieved by a reference or comparator product. In some contexts, enhanced delivery may be at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more, etc. The level of delivery of a polynucleotide (e.g., via a nanoparticle) to a target tissue can be measured, for example, by comparing the amount of protein encoded by the polynucleotide produced in the tissue per unit weight of said tissue, comparing the amount of polynucleotide in the tissue per unit weight of said tissue, comparing the amount of protein encoded by the polynucleotide produced in the tissue relative to the amount of total protein in said tissue, or comparing the amount of polynucleotide in the tissue relative to the amount of total polynucleotide in said tissue. It will be understood that enhanced delivery need not be determined in a subject being treated; it can be determined in a surrogate such as an animal model (e.g., a rat model).

As used herein, “expression” of a nucleic acid sequence refers to one or more of the following events, depending on context: (1) production of an mRNA template from a DNA sequence (e.g., by transcription); (2) processing of an mRNA transcript (e.g., by splicing, editing, 5′ cap formation, and/or 3′ end processing); (3) translation of an mRNA into a polypeptide or protein; and (4) post-translational modification of a polypeptide or protein.

As used herein, a “feature” refers to a characteristic, a property, or a distinctive element. When referring to polypeptides, “features” may include amino acid sequences and amino acid sequence-based components, such as surface manifestations, local conformational shape, folds, loops, half-loops, domains, half-domains, sites, termini, etc.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues. In some cases, the administration of a nanoparticle comprising a lipid component and an encapsulated therapeutic agent can trigger an immune response, which can be caused by (i) the encapsulated therapeutic agent (e.g., an mRNA), (ii) the expression product of such encapsulated therapeutic agent (e.g., a polypeptide encoded by the mRNA), (iii) the lipid component of the nanoparticle, or (iv) a combination thereof.

As used herein, the term “increases stability” or “increases stability of an mRNA” refers to an increase in the ability of the mRNA to resist, reduce or inhibit degradation, and/or increase or improve mRNA half-life. mRNA degradation can occur through physical (e.g., shear or UV radiation), chemical (e.g., hydrolysis), or enzymatic (e.g. nuclease activity) means. Degradation of an mRNA can occur both prior to contacting the mRNA with a cell or after contacting the mRNA with a cell. Upon contacting an mRNA with a cell, cellular machinery can induce mRNA degradation, e.g., by enzymatic cleavage of the mRNA. In some aspects, mRNA is altered to reduce susceptibility of the mRNA to enzyme-mediated degradation (e.g., exonuclease or endonuclease-mediated degradation) either prior to or following contacting the mRNA with a cell. Reducing the rate of enzymatic degradation of an mRNA in a cell can result in increased mRNA half-life or stability. In some embodiments, increased stability of an altered mRNA is measured relative to an unaltered mRNA counterpart (e.g., the starting mRNA prior to altering endonuclease sensitive motifs). In some embodiments, the unaltered mRNA counterpart is endonuclease sensitive, and altering the mRNA yields a stabilized mRNA, wherein the endonuclease resistant mRNA has an increased half-life relative to the endonuclease sensitive unaltered mRNA. The term “increases stability” within the context of a lipid or lipid delivery vehicle refers to an increase in the ability of the lipid of lipid delivery vehicle to resist, reduce, or inhibit degradation.

As used herein, the term “in vitro” refers to events that occur in an artificial environment, e.g., in a test tube or reaction vessel, in cell culture, in a Petri dish, etc., rather than within a living organism (e.g., animal, plant, or microbe).

As used herein, the term “in vivo” refers to events that occur within a living organism or (e.g., animal, plant, microbe, cell, or tissue thereof).

The term “ionizable amino lipid” (or “IAL”) includes those lipids having one, two, three, or more fatty acid or fatty alkyl chains and a pH-titratable amino head group (e.g., an alkylamino or dialkylamino head group). An ionizable amino lipid is typically protonated (i.e., positively charged) at a pH below the pKa of the amino head group and is substantially not charged at a pH above the pKa. Such ionizable amino lipids include, but are not limited to DLin-MC3-DMA (MC3) and (13Z,165Z)—N,N-dimethyl-3-nonydocosa-13-16-dien-1-amine (L608).

As used herein, the term “isolated” refers to a substance that has been separated from at least some of the components with which it was associated (whether in nature or in an experimental setting). Isolated substances may have varying levels of purity in reference to the substances from which they were isolated. Isolated substances may be separated from at least about 10%, about 20%, about 30%, about 40%, about 50%, about 60%, about 70%, about 80%, about 90%, or more of the other components from which they were isolated. In some contexts, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. By “substantially isolated” is meant that the substance is substantially separated from the environment in which it was formed or detected. Partial separation can include, for example, a composition enriched in the substance of interest.

As used herein, the term “isomer” means any tautomer, stereoisomer, enantiomer, or diastereomer of any compound of the disclosure. It is recognized that the compounds can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). According to the disclosure, the chemical structures depicted herein, and therefore the compounds of the disclosure, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the disclosure can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding one or more polypeptide(s). Translation of an mRNA, for example, in vivo translation of an mRNA inside a mammalian cell, may produce a polypeptide. Traditionally, the basic components of an mRNA molecule include at least a coding region, a 5′-untranslated region (5′UTR), a 3′UTR, a 5′ cap and a polyA sequence.

As used herein, the term “microRNA (miRNA or miR) binding site” refers to a sequence within a nucleic acid molecule, e.g., within a DNA or within an RNA transcript, including in the 5′UTR and/or 3′UTR, that has sufficient complementarity to all or a region of a miRNA to interact with, associate with or bind to the miRNA. In some embodiments, a nucleic acid molecule (e.g., RNA, e.g., mRNA) comprises an ORF encoding a polypeptide of interest and further comprises one or more miRNA binding site(s). In exemplary embodiments, a 5′UTR and/or 3′UTR of the nucleic acid molecule (e.g., RNA, e.g., mRNA) comprises the one or more miRNA binding site(s).

As used herein “modified” or “modification” refers to a changed state or a change in composition or structure of a molecule (e.g., a polynucleotide, such as mRNA). Molecules (e.g., polynucleotides) may be modified in various ways including chemically, structurally, and/or functionally. For example, polynucleotides may be structurally modified by the incorporation of one or more RNA elements, wherein the RNA element comprises a sequence and/or an RNA secondary structure(s) that provides one or more functions (e.g., translational regulatory activity). Accordingly, polynucleotides of the disclosure may be comprised of one or more modifications (e.g., may include one or more chemical, structural, or functional modifications, including any combination thereof). In some embodiments, the mRNA molecules are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

As used herein, “mucus” refers to the natural substance that is viscous and comprises mucin glycoproteins.

As used herein, “nanoparticle” refers to a particle having any one structural feature on a scale of less than about 1000 nm that exhibits novel properties as compared to a bulk sample of the same material. In some embodiments, nanoparticles have any one structural feature on a scale of less than about 500 nm, less than about 200 nm, or about 100 nm. In some embodiments, nanoparticles have any one structural feature on a scale of from about 50 nm to about 500 nm, from about 50 nm to about 200 nm or from about 70 nm to about 120 nm. In exemplary embodiments, a nanoparticle is a particle having one or more dimensions of the order of about 1 nm to about 1000 nm. In other exemplary embodiments, a nanoparticle is a particle having one or more dimensions of the order of about 10 nm to about 500 nm. In other exemplary embodiments, a nanoparticle is a particle having one or more dimensions of the order of about 50 nm to about 200 nm A spherical nanoparticle could have a diameter, for example, of between about 50 nm to about 100 nm or about 70 nm to about 120 nm. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

As disclosed herein, a “lipid nanoparticle” (LNP) refers to a nanoscale construct (e.g., less than 200 nm in diameter) comprising lipid molecules, typically arranged in a substantially spherical (e.g., spheroid) geometry. At least a portion of the LNP contains a primarily non-aqueous (e.g., lipidic) core. However, LNPs can have blebbed regions containing a primarily aqueous core, e.g., as described in Brader et al., Biophysical Journal 120: 1-5 (2021).

As used herein, the term “nuclease” refers to an enzyme with the capability to degrade or otherwise digest polynucleotides or nucleic acid molecules (e.g., DNA or RNA). Representative examples of nucleases include ribonucleases (RNase) which digests RNA, and deoxyribonuclease (DNase) which digests DNA. Unless otherwise specified, the term “nuclease” generally encompasses nuclease enzymes that are capable of degrading single-stranded polynucleotides (e.g., mRNA) and/or double stranded polynucleotides (e.g., DNA). Nucleases are a class of enzymes that are responsible for the cleavage or hydrolysis of the phosphodiester bonds that covalently link the nucleotides that comprise DNA or RNA molecules. Nucleases that cleave or hydrolyze the phosphodiester bonds of DNA are referred to herein as “deoxyribonucleases”. Nucleases that cleave the phosphodiester bonds of RNA are referred to herein as “ribonucleases”.

As used herein, the term “nucleic acid” encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a 0-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

The terms “nucleic acid sequence,” “nucleotide sequence,” or “polynucleotide sequence” are used interchangeably and refer to a contiguous nucleic acid sequence. The sequence can be either single stranded or double stranded DNA or RNA, e.g., an mRNA.

As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines (e.g., that confer improved properties such as binding affinity, nuclease resistance, chemical stability to a nucleic acid or a portion or segment thereof) Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof (e.g., that confers improved chemical and/or functional properties such as binding affinity, nuclease resistance, chemical stability to a nucleic acid or a portion or segment thereof).

As used herein, the term “open reading frame” (“ORF”), also sometimes referred to as a “coding sequence” refers to a segment or region of an mRNA molecule that encodes a polypeptide. The ORF comprises a continuous stretch of non-overlapping, in-frame codons, beginning with the initiation codon and ending with a stop codon, and can be translated by the ribosome.

As used herein, “osmolarity” refers to the measure of solute concentration, defined as the number of osmoles of solute per liter of solution (Osm/L).

As used herein, “patient” refers to a subject who may seek or be in need of treatment, requires treatment, is receiving treatment, will receive treatment, or is under care of a trained professional for a particular infection, disease, disorder, and/or condition.

The phrase “pharmaceutical composition” refers to a composition that comprises a therapeutic agent or vaccine and a pharmaceutically acceptable carrier.

As used herein, a “prophylaxis” refers to a measure taken to maintain health and prevent the onset or spread of an infection, disease, disorder and/or condition. For example, an “immune phrophylaxis” refers to a measure to produce active or passive immunity to prevent the onset or spread of an infection or disease.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified (for example by alkylation, and/or by capping) and unmodified forms of the polynucleotide. More particularly, the term “polynucleotide” includes polydeoxyribonucleotides (containing 2-deoxy-D-ribose); polyribonucleotides (containing D-ribose), including tRNA, rRNA, hRNA, siRNA and mRNA, whether spliced or unspliced; any other type of polynucleotide that is an N- or C-glycoside of a purine or pyrimidine base; and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration that allows for base pairing and base stacking, such as is found in DNA and RNA. In some aspects, the polynucleotide comprises an mRNA. In some aspects, the mRNA is a synthetic mRNA. In some aspects, the synthetic mRNA comprises at least one unnatural nucleobase. In some aspects, the synthetic mRNA comprises at least one non-standard nucleobase. In some aspects, the mRNA comprises non-natural components at positions other than the nucleobase. In some aspects, the polynucleotide (e.g., a synthetic RNA or a synthetic DNA) comprises only natural nucleobases, i.e., A (adenosine), G (guanosine), C (cytidine), and T (thymidine) in the case of a synthetic DNA, or A, C, G, and U (uridine) in the case of a synthetic RNA.

As used herein, the term “potency” refers to an amount, level, or concentration of a substance (e.g., an mRNA) that is required to produce a given response or effect.

The term “reference nucleic acid sequence” or “reference nucleic acid” or “reference nucleotide sequence” refers to a starting nucleic acid sequence (e.g., a RNA, e.g., an mRNA sequence) that can be sequence optimized. In some embodiments, the reference nucleic acid sequence is a wild type nucleic acid sequence, a fragment, or a variant thereof. In some embodiments, the reference nucleic acid sequence is a previously sequence optimized nucleic acid sequence.

As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. An RNA may be a messenger RNA (mRNA). Translation of an mRNA encoding a particular polypeptide, for example, in vivo translation of an mRNA inside a mammalian cell, may produce the encoded polypeptide. RNAs may be selected from the non-limiting group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA), and mixtures thereof.

As used herein, the term “salt” includes any anionic and cationic complex. Non-limiting examples of anions include inorganic and organic anions, e.g., fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof.

The term “sequence optimization” refers to a process or series of processes by which nucleobases in a reference nucleic acid sequence are replaced with alternative nucleobases, resulting in a nucleic acid sequence with improved properties, e.g., improved protein expression or decreased immunogenicity.

As used herein, the term “stabilize” means to make or become stable.

As used herein “stable” refers to a compound that is sufficiently robust to survive isolation to a useful degree of purity from a reaction mixture, and preferably capable of formulation into an efficacious therapeutic agent. As used herein to characterize a polynucleotide (e.g., an mRNA)), the term “stable” or “stability” refers to a reduced susceptibility to degradation or destruction (e.g., a reduced susceptibility to nuclease cleavage). For example, the term “stable” may be used to refer to a reduction in the rate of nuclease degradation (e.g., by endonuclease-mediated cleavage) of an mRNA. In certain embodiments, the half-life (t^{1/2}) of an mRNA represents an objective measurement of its stability. Similarly, in certain embodiments, the amount, expression level, or enzymatic activity of an expression product that is produced following the expression (e.g., translation) of a stable or nuclease-resistant mRNA represents an objective measurement of its stability. As used herein to characterize a delivery vehicle (e.g., lipid nanoparticle), the term “stable” or “stability” can be a function of storage conditions, and can be measured by characteristics such as consistency of size of the vehicle and polydispersity. As used herein to characterize lipids, the term “stable” or “stability” can refer to a degree of purity over time.

As used herein, the term “subject” refers to any organism to which a composition in accordance with the disclosure may be administered, e.g., for experimental, diagnostic, prophylactic, and/or therapeutic purposes. Depending on the context, “subject” also may refer to an organism from which a sample may be taken. Depending on the context, a “subject” may include animals and plants. In some contexts, a subject may be a patient.

As used herein, the term “substantially” refers to the qualitative condition of exhibiting total or near-total extent or degree of a characteristic or property. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “sustained release” refers to a pharmaceutical composition or compound release profile that conforms to a release rate over a specific period of time.

The term “synthetic” means produced, prepared, and/or manufactured by the hand of man. Synthesis of polynucleotides, polypeptides, or other molecules can be chemical or enzymatic.

As used herein, the phrase “targeting sequence” refers to a sequence that can direct the transport or localization of a protein or polypeptide.

As used herein “target tissue” refers to any one or more tissue types of interest in which the delivery of a molecules (e.g., a polynucleotide) would result in a desired biological and/or pharmacological effect. Examples of target tissues of interest include specific tissues, organs, and systems or groups thereof. In particular applications, a target tissue can be a liver, a kidney, a lung, a spleen, or a vascular endothelium in vessels (e.g., intra-coronary or intra-femoral). An “off-target tissue” refers to any one or more tissue types in which the delivery of the molecule (e.g., which results in expression of an encoded protein) does not result in a desired biological and/or pharmacological effect.

As used herein, the term “terminal region” refers to a region on the 5′ or 3′ end of a region of linked nucleosides encoding a polypeptide of interest or coding region.

The term “therapeutic agent” refers to an agent that, when administered to a subject, has a therapeutic, diagnostic, and/or prophylactic effect and/or elicits a desired biological and/or pharmacological effect. For example, in some contexts, an mRNA encoding polypeptide can be a therapeutic agent. In some contexts, a therapeutic agent may be a therapeutic protein.

As used herein, the terms “treat” or “treating” refer to an intervention that imparts a benefit to a subject afflicted with an infection, disease, disorder, and/or condition. The treating can involve partially or completely alleviating, ameliorating, improving, relieving, inhibiting progression, growth, or spread of, and/or reducing severity of a particular infection, disease, disorder, and/or condition.

As used herein, “unmodified” refers to any substance, compound or molecule prior to being changed in any way. Unmodified may, but does not always, refer to the wild type or native form of a molecule. Molecules may undergo a series of modifications whereby each modified molecule may serve as the “unmodified” starting molecule for a subsequent modification.

As used herein, the term “vaccine” refers to a biological preparation that improves immunity in the context of a particular infection, disease, disorder, and/or or condition.

As used herein, the term “variant” refers to a molecule having that has a modification relative to a reference (e.g., parent) molecule. In some embodiments, a variant is a polymorphism or an isoform. In some embodiments, the variant comprises a sequence (e.g., an amino acid or polynucleotide sequence) that is different from a reference (e.g., wild-type) sequence. In some embodiments, the variant has a sequence that is altered by substitution, insertion, or deletion, or by chemical modification as compared to a reference sequence. In some embodiments, the variant has decreased functional characteristics relative to the reference molecule. In some embodiments, the variant is functionally equivalent to the reference molecule. In some embodiments, the variant has improved functional characteristics relative to the reference molecule. Such functional characteristics can relate to stability (e.g., improved functional stability), mRNA half-life (e.g., improved mRNA half-life), mRNA potency (e.g., improved potency), mRNA resistance to endonuclease activity (e.g., reduced susceptibility to endonuclease activity). In some embodiments, the variant has at least 50%, 60%, 70%, 80%, 85%, 90%, 95%, 96%, 97%, 98%, 99% or 100% activity of the wild type molecule, e.g., as measured by an art-recognized assay.

As used herein, the term “alkyl”, “alkyl group”, or “alkylene” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C₁₋₁₄ alkyl” means an optionally substituted linear or branched, saturated hydrocarbon including 1-14 carbon atoms. Unless otherwise specified, an alkyl group described herein refers to both unsubstituted and substituted alkyl groups.

As used herein, the term “alkenyl”, “alkenyl group”, or “alkenylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C₂₋₁₄ alkenyl” means an optionally substituted linear or branched hydrocarbon including 2-14 carbon atoms and at least one carbon-carbon double bond. An alkenyl group may include one, two, three, four, or more carbon-carbon double bonds. For example, C18 alkenyl may include one or more double bonds. A C18 alkenyl group including two double bonds may be a linoleyl group. Unless otherwise specified, an alkenyl group described herein refers to both unsubstituted and substituted alkenyl groups.

As used herein, the term “alkynyl”, “alkynyl group”, or “alkynylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one carbon-carbon triple bond, which is optionally substituted. The notation “C₂₋₁₄ alkynyl” means an optionally substituted linear or branched hydrocarbon including 2-14 carbon atoms and at least one carbon-carbon triple bond. An alkynyl group may include one, two, three, four, or more carbon-carbon triple bonds. For example, C18 alkynyl may include one or more carbon-carbon triple bonds. Unless otherwise specified, an alkynyl group described herein refers to both unsubstituted and substituted alkynyl groups.

As used herein, the term “carbocycle” or “carbocyclic group” means an optionally substituted mono- or multi-cyclic system including one or more rings of carbon atoms. Rings may be three, four, five, six, seven, eight, nine, ten, eleven, twelve, thirteen, fourteen, fifteen, sixteen, seventeen, eighteen, nineteen, or twenty membered rings. The notation “C₃₋₆ carbocycle” means a carbocycle including a single ring having 3-6 carbon atoms. Carbocycles may include one or more carbon-carbon double or triple bonds and may be non-aromatic or aromatic (e.g., cycloalkyl or aryl groups). Examples of carbocycles include cyclopropyl, cyclopentyl, cyclohexyl, phenyl, naphthyl, and 1,2 dihydronaphthyl groups. The term “cycloalkyl” as used herein means a non-aromatic carbocycle and may or may not include any double or triple bond. Unless otherwise specified, carbocycles described herein refers to both unsubstituted and substituted carbocycle groups, i.e., optionally substituted carbocycles.

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. The term “heterocycloalkyl” as used herein means a non-aromatic heterocycle and may or may not include any double or triple bond. Unless otherwise specified, heterocycles described herein refers to both unsubstituted and substituted heterocycle groups, i.e., optionally substituted heterocycles.

As used herein, the term “heteroalkyl”, “heteroalkenyl”, or “heteroalkynyl”, refers respectively to an alkyl, alkenyl, alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. Unless otherwise specified, heteroalkyls, heteroalkenyls, or heteroalkynyls described herein refers to both unsubstituted and substituted heteroalkyls, heteroalkenyls, or heteroalkynyls, i.e., optionally substituted heteroalkyls, heteroalkenyls, or heteroalkynyls.

As used herein, an “aryl group” is an optionally substituted carbocyclic group including one or more aromatic rings. Examples of aryl groups include phenyl and naphthyl groups. As used herein, a “heteroaryl group” is an optionally substituted heterocyclic group including one or more aromatic rings. Examples of heteroaryl groups include pyrrolyl, furyl, thiophenyl, imidazolyl, oxazolyl, and thiazolyl. Both aryl and heteroaryl groups may be optionally substituted. For example, M and M′ can be selected from the non-limiting group consisting of optionally substituted phenyl, oxazole, and thiazole. In the formulas herein, M and M′ can be independently selected from, but not limited to, —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group. Unless otherwise specified, aryl or heteroaryl groups described herein refers to both unsubstituted and substituted groups, i.e., optionally substituted aryl or heteroaryl groups.

Alkyl, alkenyl, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., C(O)OH), an alcohol (e.g., a hydroxyl, OH), an ester (e.g., C(O)OR OC(O)R), an aldehyde (e.g., C(O)H), a carbonyl (e.g., C(O)R, alternatively represented by C═O), an acyl halide (e.g., C(O)X, in which X is a halide selected from bromide, fluoride, chloride, and iodide), a carbonate (e.g., OC(O)OR), an alkoxy (e.g., OR), an acetal (e.g., C(OR)₂R″, in which each OR are alkoxy groups that can be the same or different and R″ is an alkyl or alkenyl group), a phosphate (e.g., P(O)₄³⁻), a thiol (e.g., SH), a sulfoxide (e.g., S(O)R), a sulfinic acid (e.g., S(O)OH), a sulfonic acid (e.g., S(O)₂OH), a thial (e.g., C(S)H), a sulfate (e.g., S(O)₄²⁻), a sulfonyl (e.g., S(O)₂), an amide (e.g., C(O)NR₂, or N(R)C(O)R), an azido (e.g., N₃), a nitro (e.g., NO₂), a cyano (e.g., CN), an isocyano (e.g., NC), an acyloxy (e.g., OC(O)R), an amino (e.g., NR₂, NRH, or NH₂), a carbamoyl (e.g., OC(O)NR₂, OC(O)NRH, or OC(O)NH₂), a sulfonamide (e.g., S(O)₂NR₂, S(O)₂NRH, S(O)₂NH₂, N(R)S(O)₂R, N(H)S(O)₂R, N(R)S(O)₂H, or N(H)S(O)₂H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. For example, a C₁₋₆ alkyl group may be further substituted with one, two, three, four, five, or six substituents as described herein.

As used herein, the term “compound,” is meant to include all stereoisomers and isotopes of the structure depicted. As used herein, the term “stereoisomer” means any geometric isomer (e.g., cis- and trans-isomer), enantiomer, or diastereomer of a compound. The present disclosure encompasses any and all stereoisomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known. “Isotopes” refers to atoms having the same atomic number but different mass numbers resulting from a different number of neutrons in the nuclei. For example, isotopes of hydrogen include tritium and deuterium. Further, a compound, salt, or complex can be prepared in combination with solvent or water molecules to form solvates and hydrates by routine methods.

As used herein, the term “diastereomer,” means stereoisomers that are not mirror images of one another and are non-superimposable on one another.

As used herein, the term “enantiomer” means each individual optically active form of a compound, having an optical purity or enantiomeric excess (as determined by methods standard in the art) of at least 80% (i.e., at least 90% of one enantiomer and at most 10% of the other enantiomer), at least 90%, or at least 98%.

### EXAMPLES

### Example 1. Purification of Compound A (IAL)

### Alkyl Halide Scavenging: DABCO

Following synthesis of Compound A (IAL), a crude IAL composition was analyzed by UPLC-UV to quantify the amount of Compound B-iodide present in the crude IAL composition. Briefly, an aliquot of the crude IAL solution was pipetted into a 20 mL glass vial, and insoluble solids were allowed to settle to the bottom of the flask. A 100-μL aliquot of the supernatant (approximately 50 mg/mL) was decanted and diluted with 400 μL isopropanol to yield a 10 mg/mL solution. This sample was analyzed using a quantitative HPLC method for determining residual iodide to determine the initial concentration of Compound B-iodide in the crude IAL composition.

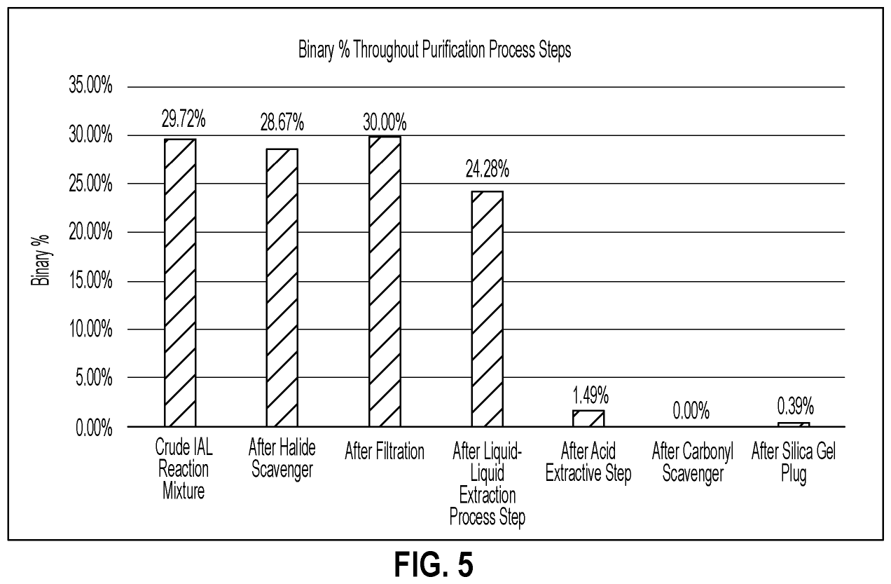

To remove Compound B-iodide, DABCO was added to the crude IAL composition at a stoichiometric excess to the halide impurities, and the reaction mixture was stirred at 75-80° C. for four hours. Aliquots of the reaction solution were removed hourly and analyzed for residual iodide. Referring to Table 1 and FIG. 5, residual iodide concentration was reduced to non-detectable levels (ND) after 4 hours. The reaction was allowed to proceed at ambient temperature overnight.

TABLE 1
Concentration of Residual Compound
B-iodide Versus Scavenging Time

| Time (hours) | Compound B-iodide (ppm) |
|---|---|
| 0 | 2234 |
| 1 | 225 |
| 2 | 30 |
| 3 | 14 |
| 4 | ND |

The reaction solution was removed from stirring and filtered through a CELITE® pad in a disposable PTFE filter funnel to remove insoluble inorganic salts. The reaction vessel was rinsed with acetonitrile and that rinse was passed through the CELITE® pad.

### Concentration

The combined filtrates obtained as described above were concentrated in vacuo at 40° C. to yield an oil with some white particulates (e.g., unreacted reagents, inorganic salts, etc.). This neat material was azeotroped with heptane at 40° C. The resulting neat material was diluted in heptane (3 vol. eq.) to yield a white suspension. To remove the white particulates, the suspension was filtered through a fresh CELITE® pad in a disposable PTFE filter funnel. The flask was rinsed with two volume equivalents of heptane a total of two times (a total of four volume equivalents heptane). Each rinsate was passed through the CELITE® pad, and the filtrates were combined.

### Heptane/Acetonitrile/Methanol Wash

The combined heptane filtrate obtained as described above (including 7 vol. eq. heptane) was transferred to a separatory funnel and washed with a 2:1 solution of MeCN:MeOH (3 vol. eq.), three times. Two layers quickly formed during each wash. After the third MeCN/MeOH wash, the heptane layer was washed with 1:1 MeCN:water (3 vol. eq.), followed by MeCN only (3 vol. eq.).

### Malonic Acid Extraction

The heptane layer was washed with a solution of malonic acid (1.25 eq., relative to Compound A) in 4:1 MeCN:MeOH (5 vol. eq.) to convert the Compound A (IAL) into a malonate salt that is soluble in MeCN. After the two layers separated, the MeCN/MeOH layer was washed with heptane (3 vol. eq.), two times.

### Aldehyde Scavenging: 2-(Aminooxy)Acetic Acid Hemihydrochloride Treatment

The MeCN/MeOH layer containing Compound A-malonate salt was transferred to an Erlenmeyer flask equipped with a magnetic stir bar. 2-(aminooxy)acetic acid hemihydrochloride was added at a stoichiometric excess to the aldehyde impurities. The solution was then stirred for 2 hours at ambient temperature. After the reaction had been stirred for 2 hours, the solution was transferred back into a separatory funnel where it was washed with heptane (3 vol. eq.) to remove residual nonpolar impurities (such as alkyl halides that did not react with the halide scavenger (DABCO) or aldehydes that did not react with the aldehyde scavenger (2-(aminooxy)acetic acid hemihydrochloride)).

### Neutralization

The MeCN/MeOH solution containing the Compound A-malonate salt was transferred to an Erlenmeyer flask equipped with a magnetic stir bar. A solution of K₃PO₄ (2.5 mol. eq., relative to Compound A-malonate) in water (5 vol. eq.) was added to the flask in one portion. The reaction mixture was stirred for one hour before heptane (5 vol. eq.) was added to the flask in one portion. The biphasic solution was removed from stirring and transferred to a 2 L separatory funnel, where two layers separated quickly. The heptane layer was washed with MeCN (4 vol. eq.) to remove residual malonate salt and was partially distilled to half volume to remove trace amounts of MeCN, MeOH, and water. The resulting heptane solution containing purified Compound A (IAL) was then diluted by a factor of 10 by volume, using heptane.

### Silica Gel Plug

Loose silica gel was mixed into a slurry with an adequate amount of heptane to ensure even dispersion in a disposable PTFE filter funnel. A vacuum was then pulled through the funnel to allow even settling of the silica gel without completely drying the silica gel (a small amount of heptane remained above the plug). The silica gel plug was then covered with a sheet of filter paper. The heptane solution was then filtered through the silica gel plug without drying out the plug. The plug was then washed with heptane (5 vol. eq.) and isopropanol (5 vol. eq.). The filtrate was then concentrated in vacuo at 40° C.

### Lipid-mRNA Adduct Formation as a Function of Process Step

After various process steps discussed above, an aliquot of the resulting IAL-containing composition was obtained, combined with mRNA, and the extent of lipid-mRNA adduct formation was determined by reverse phase ion pair chromatography (RP-IP HPLC), according to a lipid binary assay as described in U.S. application Ser. No. 17/508,786 and Packer et al., Nature Comm. (2021) 12: 6777. FIG. 5 shows the extent of adduct formation (as binary %) after the indicated steps. The data shows approximately the same extent of adduct formation after halide scavenging, filtration, and liquid-liquid extraction as was observed for the crude IAL composition. However, the extent of adduct formation is significantly reduced after acid extraction, and is even further reduced after carbonyl scavenging and removal of polar impurities by silica gel plug. Decreased formation of lipid-mRNA adducts after successive steps in the purification process demonstrates improved IAL purity after halide scavenging-removal, acid extraction, and carbonyl scavenging as described herein.

### Example 2. Lipid-mRNA Adduct Formation for Combinations of Halide and Aldehyde Scavenging Steps

To assess improvements in IAL purity obtained by individual scavenging-removal steps of a process as described herein, the extent of lipid-mRNA adduct formation observed in compositions prepared by combining mRNA with an IAL composition obtained by various steps of a process according to FIG. 1 was determined. In particular, IAL compositions obtained by a process comprising the aldehyde-scavenging step but not the halide scavenging step (“Aldehyde Scavenger Only”), a process comprising the halide scavenging step but not the aldehyde scavenging step (“Halide Scavenger Only”), a process comprising all steps (“Halide and Aldehyde Scavenger”), and a process omitting the halide scavenging and aldehyde scavenging steps (“No Scavengers”) were assessed. Adduct formation was measured using reverse phase ion pair chromatography (RP-IP HPLC), according to the lipid binary assay as described in U.S. application Ser. No. 17/508,786 and Packer et al., Nature Comm. (2021) 12: 6777. A reduction in lipid-mRNA adduct formation indicates improved IAL purity.

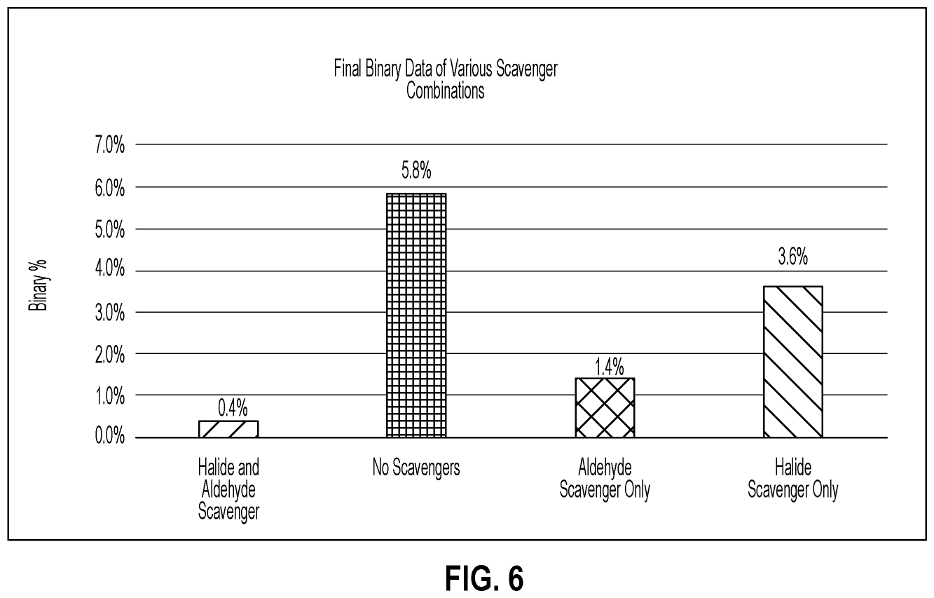

As shown in FIG. 6, performing a halide scavenger step and/or an aldehyde scavenger step significantly reduces lipid-mRNA adduct formation when compared to a process using no scavenging steps. A process using both alkyl halide scavenging and aldehyde scavenging showed the lowest adduct formation (indicating the highest IAL purity).

What is claimed is:

1. A method of purifying an ionizable amino lipid (IAL) present in a first composition comprising the IAL, a first reactive impurity, and a second reactive impurity in a first nonpolar solvent, the method comprising:
(a) performing a first scavenging-removal step, comprising:
contacting the first composition with a first scavenger, wherein the first scavenger reacts with the first reactive impurity to form a first scavenger-impurity product; and
separating the first scavenger-impurity product from the first composition to obtain a second composition comprising the IAL and the second reactive impurity;
(b) performing an acid extraction step, comprising contacting the second composition with an acid, wherein the acid reacts with the IAL to form an IAL salt that is soluble in a polar solvent, and transferring the IAL salt and the second reactive impurity into the polar solvent to obtain an IAL salt solution;
(c) performing a second scavenging-removal step, comprising:
contacting the IAL salt solution with a second scavenger, wherein the second scavenger reacts with the second reactive impurity to form a second scavenger-impurity product;
converting the IAL salt into the IAL with a neutralizing agent, wherein the IAL is soluble in a second nonpolar solvent; and
separating the IAL from the second scavenger-impurity product, or a derivative thereof formed by the neutralizing agent, by transferring the IAL into the second nonpolar solvent to obtain a purified IAL composition comprising purified IAL.

2. A method of purifying an ionizable amino lipid (IAL) present in a first composition comprising the IAL, a reactive halide impurity, and a reactive aldehyde impurity in a first nonpolar solvent, the method comprising:
(a) performing a halide scavenging-removal step, comprising:
contacting the first composition with a halide scavenger that reacts with the reactive halide impurity to form a scavenger-halide product; and
separating the scavenger-halide product from the first composition to obtain a second composition comprising the IAL and reactive aldehyde impurity;
(b) performing an acid extraction step, comprising contacting the second composition with an acid that reacts with the IAL to form an IAL salt that is soluble in a polar solvent, and transferring the IAL salt and reactive aldehyde impurity into the polar solvent to obtain an IAL salt solution;
(c) performing an aldehyde scavenging-removal step, comprising:
contacting the IAL salt solution with an aldehyde scavenger that reacts with the reactive aldehyde impurity to form a scavenger-aldehyde product;
converting the IAL salt into the IAL with a neutralizing agent, wherein the IAL is soluble in a second nonpolar solvent; and
separating the IAL from the scavenger-aldehyde product, or a derivative thereof formed by the neutralizing agent, by transferring the IAL into the second nonpolar solvent to obtain a purified IAL composition comprising purified IAL.

3. The method of claim 2, wherein the scavenger-aldehyde product or derivative thereof is more soluble in a second polar solvent than in the second nonpolar solvent, and wherein the separating further comprises transferring the scavenger-aldehyde product or derivative thereof into the second polar solvent.

4. The method of claim 3, wherein the neutralizing agent forms a derivative of the scavenger-aldehyde product that is a salt of the scavenger-aldehyde product that is more soluble in the second polar solvent than in the second nonpolar solvent, and wherein the separating comprises transferring the salt of the scavenger-aldehyde product into the second polar solvent.

5. The method of claim 3 or claim 4, wherein the scavenger-aldehyde product is more soluble in the second polar solvent than in the second nonpolar solvent, and wherein the separating further comprises transferring the second scavenger-impurity product into the second polar solvent.

6. The method of any one of claims 2-5, wherein the halide scavenger comprises one or more selected from amine compounds, phosphine compounds, thiol compounds, and sulfoxide compounds.

7. The method of any one of claims 2-6, wherein the halide scavenger comprises one or more selected from triphenylphosphine, tributylphosphine, triethylamine, triethylenediamine (DABCO), 4-dimethylaminopyridine (DMAP), dimethylsulfoxide, and dodecanethiol.

8. The method of any one of claims 2-7, wherein the halide scavenger comprises one or more selected from tributylphosphine, triethylamine, triethylenediamine (DABCO), and 4-dimethylaminopyridine (DMAP).

9. The method of any one of claims 2-8, wherein the halide scavenger comprises triethylenediamine (DABCO).

10. The method of any one of claims 2-9, wherein the scavenger-halide product is a quaternary ammonium compound.

11. The method of any one of claims 2-10, wherein separating the scavenger-halide product from the first composition comprises performing a liquid-liquid extraction.

12. The method of any one of claims 2-11, wherein the aldehyde scavenger comprises one or more selected from O-benzylhydroxylamine (O-BHA), N-benzylhydroxylamine, 2-(aminooxy)acetic acid, O-tritylhydroxylamine, O-(2,3,4,5,6-pentafluorobenzyl)hydroxylamine (PFBHA), p-benzyloxybenzyl alcohol-based hydroxylamines and salts thereof, aminobenzamides, and cysteines and cysteine analogs.

13. The method of any one of claims 2-12, wherein the aldehyde scavenger comprises one or more selected from O-benzylhydroxylamine (O-BHA), N-benzylhydroxylamine, and 2-(aminooxy)acetic acid.

14. The method of any one of claims 2-13, wherein the aldehyde scavenger comprises 2-(aminooxy)acetic acid.

15. The method of any one of claims 2-14, wherein the scavenger-aldehyde product comprises a compound selected from a Schiff base and an oxime compound.

16. The method of any one of claims 1-15, wherein the first nonpolar solvent comprises an alkane.

17. The method of any one of claims 1-16, wherein the first nonpolar solvent comprises heptane.

18. The method of any one of claims 1-17, wherein the second nonpolar solvent comprises an alkane.

19. The method of any one of claims 1-18, wherein the second nonpolar solvent comprises heptane.

20. The method of any one of claims 1-19, wherein the first polar solvent comprises acetonitrile, methanol, or a combination thereof.

21. The method of any one of claims 2-20, wherein the second polar solvent comprises acetonitrile, methanol, or a combination thereof.

22. The method of any one of claims 1-21, wherein the acid comprises one or more selected from carboxylic acids, inorganic acids, and sulfonic acids.

23. The method of any one of claims 1-22, wherein the acid comprises one or more selected from ethylmalonic acid, malonic acid, methanesulfonic acid, methylmalonic acid, and phosphoric acid.

24. The method of any one of claims 1-23, wherein the acid comprises malonic acid.

25. The method of any one of claims 2-24, wherein the contacting of the halide scavenging-removal step comprises adding the halide scavenger to the first composition and stirring at a temperature of from 60° C. to 90° C. for from 2 hours to 6 hours.

26. The method of any one of claims 2-25, wherein the halide scavenging-removal step further comprises, after the contacting and before the separating, filtering the composition to remove inorganic salts.

27. The method of any one of claims 2-26, wherein the halide scavenging-removal step further comprises, after the contacting and before the separating, concentrating the first composition.

28. The method of any one of 2-27, wherein the separating of the halide scavenging-removal step comprises washing the first composition with a polar wash solvent to obtain the second composition.

29. The method of claim 28, wherein the polar wash solvent comprises acetonitrile, methanol, or a combination thereof.

30. The method of any one of claims 2-29, further comprising removing nonpolar impurities by washing the IAL salt solution with a nonpolar wash solvent before performing the aldehyde scavenging-removal step.

31. The method of claim 30, wherein the nonpolar wash solvent for washing the IAL salt solution before performing the aldehyde scavenging-removal step comprises heptane.

32. The method of any one of claims 2-31, wherein the contacting of the aldehyde scavenging-removal step comprises adding the aldehyde scavenger to the IAL salt solution and stirring at a temperature of 20° C. to 50° C. for from 15 minutes to 4 hours.

33. The method of any one of claims 2-32, wherein the aldehyde scavenging-removal step further comprises, after the contacting and before the converting, washing the IAL salt solution comprising the aldehyde-scavenger product with a nonpolar wash solvent.

34. The method of claim 33, wherein the nonpolar wash solvent for washing the IAL salt solution, after the contacting and before the converting, comprises heptane.

35. The method of any one of claims 2-34, wherein the separating of the aldehyde scavenging-removal step comprises washing the second nonpolar solvent containing the IAL with a polar wash solvent to obtain the purified IAL composition comprising purified IAL.

36. The method of claim 35, wherein the polar wash solvent for washing the second nonpolar solvent containing the IAL comprises one or more of acetonitrile and methanol.

37. The method of any one of claims 1-36, further comprising filtering the purified IAL composition to remove residual impurities.

38. The method of claim 37, wherein the filtering comprises passing the purified IAL composition through a silica gel plug.

39. The method of any one of claims 1-38, further comprising concentrating the purified IAL composition.

40. The method of claim 4, wherein the salt of the scavenger-aldehyde product is an oxime salt.

41. A method of purifying an ionizable amino lipid (IAL) present in a first composition comprising the IAL and a reactive halide impurity in a nonpolar solvent, the method comprising performing a halide scavenging-removal step, comprising:
contacting the first composition with a scavenger that reacts with the reactive halide impurity to form a scavenger-halide product; and
separating the scavenger-halide product from the first composition to obtain a purified IAL composition comprising purified IAL.

42. A method of purifying an ionizable amino lipid (IAL) present in a composition comprising the IAL and a reactive aldehyde impurity in a first nonpolar solvent, the method comprising:
performing an acid extraction step, comprising contacting the composition with an acid that reacts with the IAL to form an IAL salt that is soluble in a polar solvent, and transferring the IAL salt and reactive aldehyde impurity into the polar solvent to obtain an IAL salt solution; and
performing an aldehyde scavenging-removal step, comprising:
contacting the IAL salt solution with an aldehyde scavenger that reacts with the reactive aldehyde impurity to form a scavenger-aldehyde product;
converting the IAL salt into the IAL with a neutralizing agent, wherein the IAL is soluble in a second nonpolar solvent; and
separating the IAL from the scavenger-aldehyde product, or a derivative thereof formed by the neutralizing agent, by transferring the IAL into the second nonpolar solvent to obtain a purified IAL composition comprising purified IAL.

43. The method of claim 1, wherein the derivative of the second scavenger-impurity product is a salt of the scavenger-impurity product.

44. The method of any one of claims 1-43, wherein the IAL comprises a compound according to Formula (I): 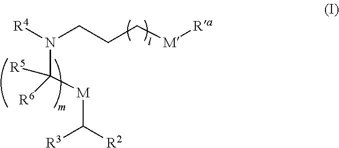
or isomer thereof, wherein R′^{a} is R′^{branched}; wherein R′^{branched} is: 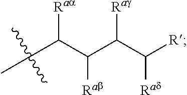
wherein [symbol] denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and 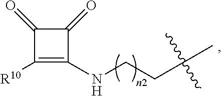
wherein [symbol] denotes a point of attachment;
wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

45. The method of any one of claims 1-43, wherein the IAL is selected from:

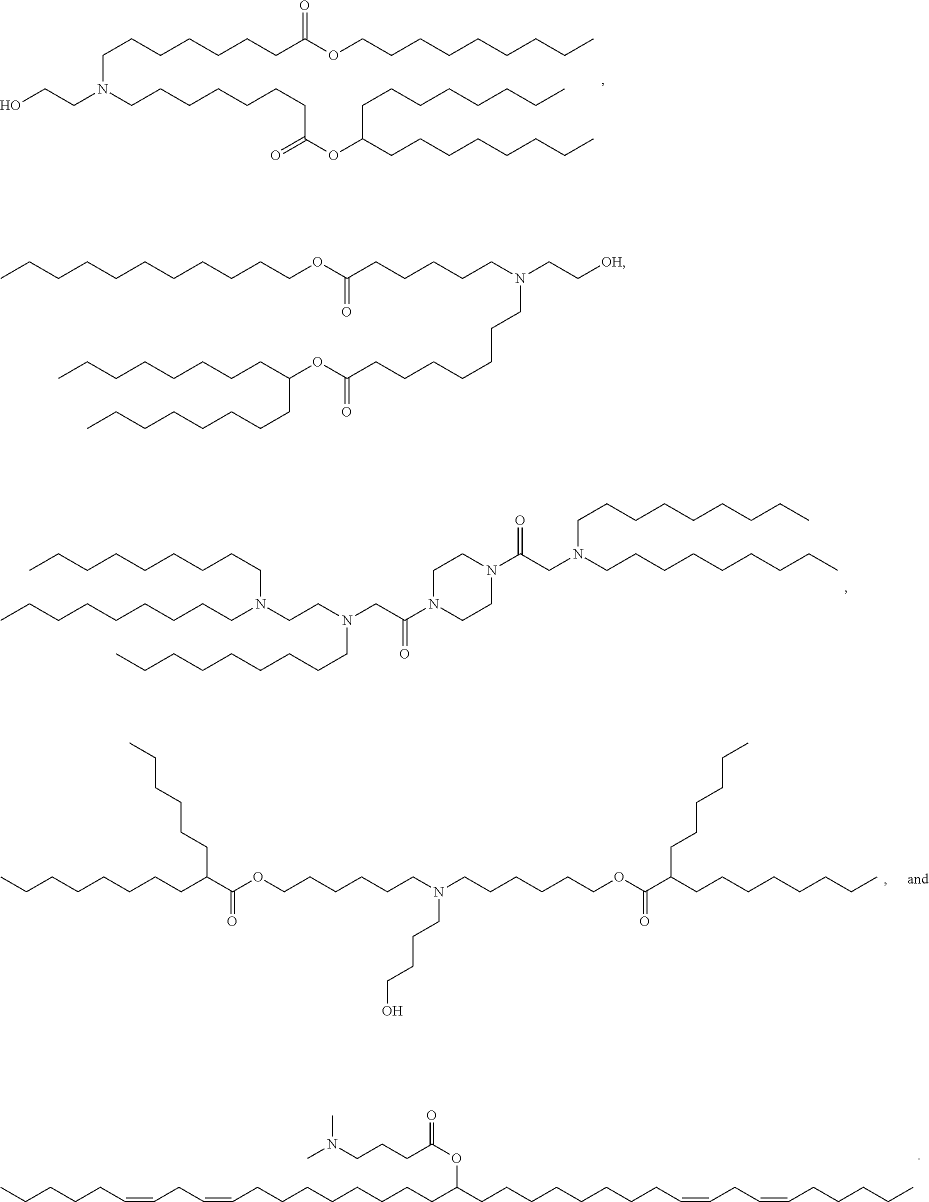

46. The method of any one of claims 1-45, wherein the method does not comprise performing column chromatography.

47. A purified ionizable amino lipid (IAL) obtained by the method according to any one of claims 1-46.

48. A lipid nanoparticle (LNP) comprising a purified ionizable amino lipid obtained by the method according to any one of claims 1-46.

49. The LNP according to claim 48, wherein the LNP further comprises a polynucleotide.

50. The LNP according to claim 49, wherein the polynucleotide is mRNA.

51. The LNP according to any one of claims 48-50, further comprising a phospholipid, cholesterol, and a PEG-lipid.

52. The LNP according to claim 51, comprising a molar ratio of 20-60% IAL, 5-25% phospholipid, 25-55% cholesterol, and 0.5-15% PEG-lipid, based on lipid components.

53. A pharmaceutical composition comprising a lipid nanoparticle (LNP), wherein the LNP comprises a purified ionizable amino lipid obtained by the method according to any one of claims 1-46.

54. The pharmaceutical composition according to claim 53, wherein the LNP further comprises a polynucleotide.

55. The pharmaceutical composition according to claim 54, wherein the polynucleotide is mRNA.

56. The pharmaceutical composition according to any one of claims 53-55, wherein the LNP further comprises a phospholipid, cholesterol, and a PEG-lipid.

57. The LNP according to claim 56, comprising a molar ratio of 20-60% IAL, 5-25% phospholipid, 25-55% cholesterol, and 0.5-15% PEG-lipid, based on lipid components.

58. A pharmaceutical composition, comprising:
a lipid nanoparticle comprising a mRNA, a phospholipid, a cholesterol, and a PEG-lipid, and a purified ionizable amino lipid (IAL) obtained by the method according to any one of claims 1-46.

59. The pharmaceutical composition of claim 58, wherein less than about 10% of the mRNA is in the form of an ionizable lipid-polynucleotide adduct impurity, optionally wherein less than about 5% of the mRNA is in the form of an ionizable lipid-polynucleotide adduct impurity, optionally wherein less than about 1% of the mRNA is in the form of an ionizable lipid-polynucleotide adduct impurity, optionally wherein the pharmaceutical composition is substantially free of ionizable lipid-polynucleotide adduct impurity, as measured by reverse phase ion pair high performance liquid chromatography (RP-IP HPLC)

60. The composition of claim 58 or claim 59, wherein the IAL is selected from:

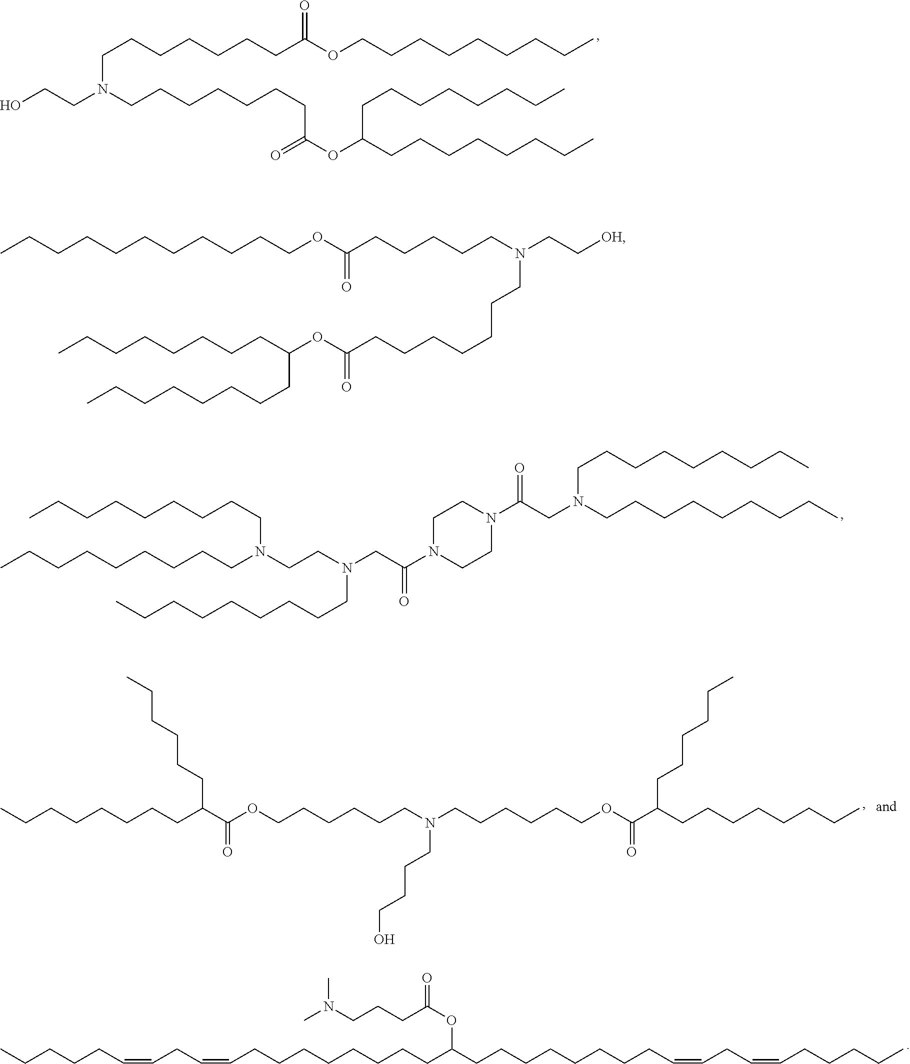

61. The composition of any one of claims 58-60, wherein the composition comprises a buffer selected from the group consisting of sodium phosphate, sodium citrate, sodium succinate, histidine, histidine-HCl, sodium malate, sodium carbonate, and Tris (tris(hydroxymethyl)aminomethane).

62. The composition of any one of claims 58-61, wherein the composition comprises a cryoprotectant.

63. The composition of claim 62, wherein the cryoprotectant is selected from the group consisting of: mannitol; sucrose; trehalose; lactose; glycerol; dextrose; and combinations thereof.

64. The composition of any one of claims 58-63, wherein the ionizable lipid-polynucleotide adduct impurity comprises an aldehyde-mRNA adduct impurity.

65. The composition of any one of claims 58-64, wherein an amount of lipid aldehydes in the composition is less than about 50 ppm.

66. The composition of any one of claims 58-65, wherein the composition comprises Tris buffer and sucrose.

67. The composition of any one of claims 58-66, wherein the composition comprises a molar ratio of 20-60% IAL, 5-25% phospholipid, 25-55% cholesterol, and 0.5-15% PEG-lipid, based on lipid components.

68. A composition comprising a lipid nanoparticle comprising a mRNA, a phospholipid, a cholesterol, a PEG-lipid, and an IAL comprising a tertiary amine group obtained by the method according to any one of claims 1-46,
wherein less than about 10% of the mRNA is in the form of an ionizable lipid-polynucleotide adduct impurity, optionally wherein less than about 5% of the mRNA is in the form of an ionizable lipid-polynucleotide adduct impurity, optionally wherein less than about 1% of the mRNA is in the form of am ionizable lipid-polynucleotide adduct impurity, optionally wherein the pharmaceutical composition is substantially free of ionizable lipid-polynucleotide adduct impurity, as measured by reverse phase ion pair high performance liquid chromatography (RP-IP HPLC).

69. The composition of claim 68, wherein the IAL is selected from:

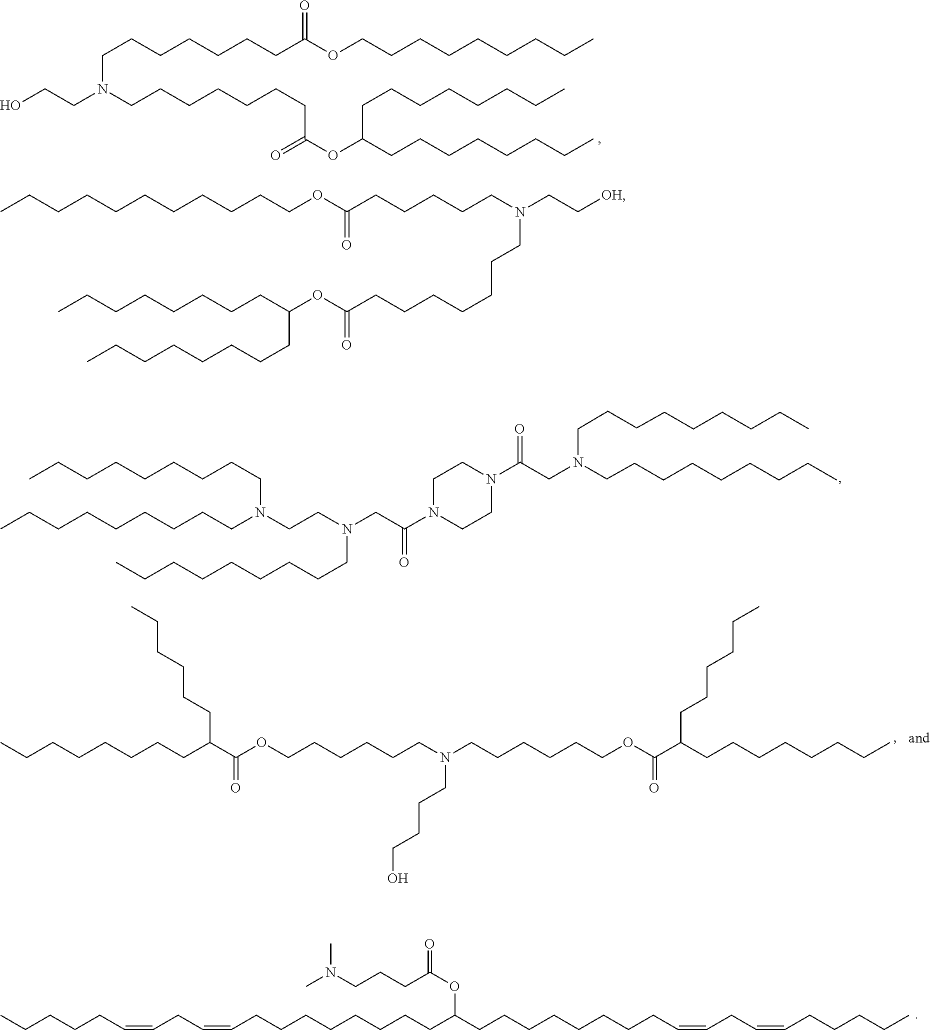

70. The composition of claim 68 or claim 69, wherein the composition comprises a buffer selected from the group consisting of sodium phosphate, sodium citrate, sodium succinate, histidine, histidine-HCl, sodium malate, sodium carbonate, and Tris (tris(hydroxymethyl)aminomethane).

71. The composition of any one of claims 68-70, wherein the composition comprises a cryoprotectant.

72. The composition of claim 71, wherein the cryoprotectant is selected from the group consisting of: mannitol; sucrose; trehalose; lactose; glycerol; dextrose; and combinations thereof.

73. The composition of any one of claims 68-72, wherein the ionizable lipid-polynucleotide adduct impurity comprises an aldehyde-mRNA adduct impurity.

74. The composition of any one of claims 68-73, wherein an amount of lipid aldehydes in the composition is less than about 50 ppm.

75. The composition of any one of claims 68-74, wherein the composition comprises Tris buffer and sucrose.

76. The composition of any one of claims 68-75, wherein the composition comprises a molar ratio of 20-60% IAL, 5-25% phospholipid, 25-55% cholesterol, and 0.5-15% PEG-lipid, based on lipid components.

77. A composition comprising a lipid nanoparticle comprising a mRNA, a phospholipid, a cholesterol, a PEG-lipid, and an IAL obtained by the method according to any one of claims 1-46, wherein the ionizable lipid is selected from: 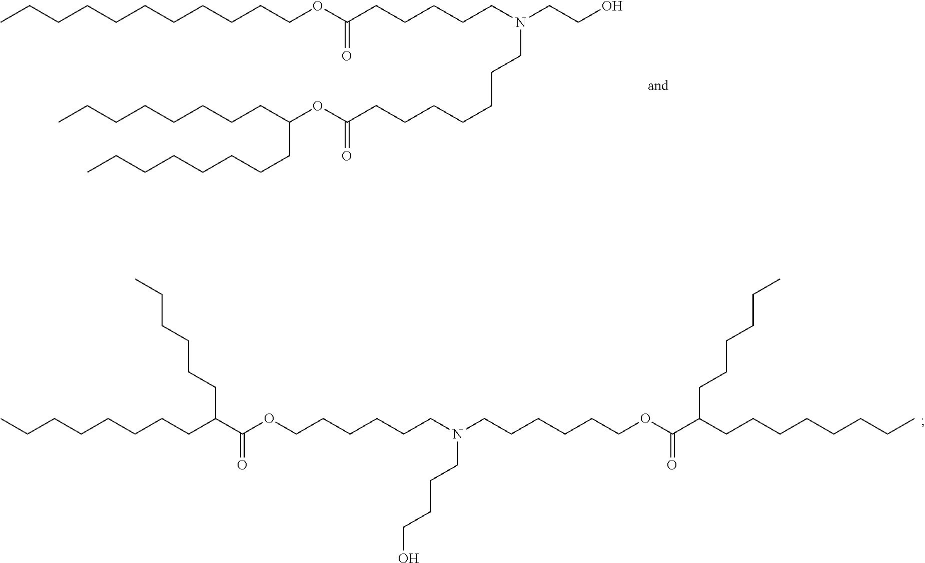 [SMILES: CCCCCCCCCCCOC(=O)CCCCCN(CCO)CCCCCCCC(=O)OC(CCCCCCCC)CCCCCCCC ; CCCCCCCCC(CCCCCC)C(=O)OCCCCCCN(CCCCO)CCCCCCOC(=O)C(CCCCCC)CCCCCCCC]
wherein the composition comprises a Tris buffer and sucrose; and
wherein less than about 10% of the mRNA is in the form of an ionizable lipid-polynucleotide adduct impurity, optionally wherein less than about 5% of the mRNA is in the form of the ionizable lipid-polynucleotide adduct impurity, optionally wherein less than about 1% of the mRNA is in the form of the ionizable lipid-polynucleotide adduct impurity, optionally wherein the pharmaceutical composition is substantially free of ionizable lipid-polynucleotide adduct impurity, as measured by reverse phase ion pair high performance liquid chromatography (RP-IP HPLC).
